(19) United States
(12) Patent Application Publication
(10) Pub. No.: US 2024/0230683 A1
(43) Pub. Date: Jul. 11, 2024

(54) NF-KB AS BIOMARKER FOR ASSESSING TREATMENT EFFICACY IN PARKINSON'S DISEASE

(71) Applicant: Board of Regents of the University Of Nebraska, Lincoln, NE (US)

(72) Inventors: Howard GENDELMAN, Omaha, NE (US); R. Lee MOSLEY, Omaha, NE (US); Mai MOSTAFA, Omaha, NE (US); Katherine OLSON, Omaha, NE (US)

(73) Assignee: Board of Regents of the University Of Nebraska, Lincoln, NE (US)

(21) Appl. No.: 18/560,365

(22) PCT Filed: May 13, 2022
(86) PCT No.: PCT/US2022/029318

§ 371 Date: Nov. 10, 2023

Related U.S. Application Data
(60) Provisional application No. 63/188,661, filed on May 14, 2021.

(51) Int. Cl. G01N 33/68; A61K 31/198; A61K 38/17; A61K 38/19; A61K 38/22; A61P 25/16

(52) U.S. Cl. CPC G01N 33/6896; A61K 31/198; A61K 38/1709; A61K 38/193; A61K 38/2278; A61P 25/16; G01N 2333/4703; G01N 2333/916; G01N 2800/2835; G01N 2800/52

(57) ABSTRACT

Provided are methods for monitoring the progression of Parkinson's Disease, and methods for monitoring or determining the effectiveness of therapeutics for the treatment of Parkinson's Disease, as well as methods for treatment thereof, by assessing one or more biomarkers, such as NF-κB and/or calcineurin.

### CROSS-REFERENCE TO RELATED APPLICATIONS

This application claims priority to U.S. Provisional Patent Application No. 63/188,661, filed May 14, 2021, entitled “Biomarkers for Parkinson's Disease,” which is herein incorporated by reference in its entirety for all purposes.

### STATEMENT REGARDING FEDERALLY SPONSORED RESEARCH OR DEVELOPMENT

This invention was made with Government support under Grant No. R01NS034239 from the National Institutes of Health. The Government has certain rights in this invention.

### FIELD

The present disclosure relates in some aspects to methods for monitoring the progression of Parkinson's Disease, and methods for monitoring or determining the effectiveness of therapeutics for the treatment of Parkinson's Disease, as well as methods for treatment thereof, by assessing one or more biomarkers, such as NF-κB and/or calcineurin.

### BACKGROUND

There is difficulty with developing biomarkers for PD due to the heterogeneity of disease and significant overlap of the clinical and biochemical features of PD with other neurodegenerative disorders. So far, there is no definitive biomarker to evaluate the response to new therapies for PD. This highlights the need to identify biomarkers that can be measured during the treatment course to assess the response of subjects, e.g., patients, to the therapy. The present invention addresses this need.

### SUMMARY

Provided herein is a method of determining the efficacy of a therapeutic agent for the treatment of Parkinson's Disease (PD), the method comprising: a) determining a baseline expression level of NF-kB and/or calcineurin in a biological sample from a subject having PD; b) determining an expression level of NF-kB and/or calcineurin in a biological sample from the subject at one or more time points following initiation of treatment with a therapeutic agent; and c) determining if the expression level of NF-kB and/or calcineurin at the one or more time points following initiation of treatment decreased compared to the baseline expression level.

Also provided herein is a method of determining the efficacy of a therapeutic agent for the treatment of Parkinson's Disease (PD), the method comprising: a) determining a baseline expression level of NF-kB and/or calcineurin in a biological sample from a subject having PD; b) determining an expression level of NF-kB and/or calcineurin in a biological sample from a subject at one or more time points following initiation of treatment with a therapeutic agent; and c) comparing the expression level of NF-kB and/or calcineurin at the one or more time points following initiation of treatment to the baseline expression level, wherein the comparison indicates a likelihood of therapeutic efficacy for the treatment of PD in the subject and/or indicates a degree of effectiveness at treating PD in the subject.

In some embodiments, the comparison indicates that the expression level of NF-kB and/or calcineurin at the one or more time points following initiation of treatment decreased compared to the baseline expression level.

In some embodiments, if the expression level of NF-kB and/or calcineurin at the one or more time points following initiation of treatment decreased compared to the baseline expression level, the comparison indicates an increased likelihood of therapeutic efficacy for the treatment of PD in the subject and/or indicates an increased degree of effectiveness at treating PD in the subject. In some embodiments, if the expression level of NF-kB and/or calcineurin at the one or more time points following initiation of treatment did not decrease compared to the baseline expression level, the comparison indicates a neutral or decreased likelihood of therapeutic efficacy for the treatment of PD in the subject and/or indicates a neutral or decreased degree of effectiveness at treating PD in the subject.

In some of any of such embodiments, a decreased expression level of NF-kB and/or calcineurin at the one or more time points following initiation of treatment compared to the baseline expression level indicates an increase in likelihood of therapeutic efficacy for the treatment of PD in the subject and/or indicates an increase in degree of effectiveness at treating PD in the subject.

In some of any of such embodiments, the baseline expression level is determined at or at least 1 day, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment.

In some of any of such embodiments, the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment. In some of any of such embodiments, the one or more time points following initiation of treatment with the therapeutic agent comprises a time point at or about 2 months following initiation of treatment. In some of any of such embodiments, the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.

In some of any of such embodiments, the expression level of NF-kB and/or calcineurin is determined by an assay. In some embodiments, the assay is selected from the group consisting of mass spectrometry, enzyme-linked immunosorbent assay (ELISA), western blotting, or polymerase chain reaction (PCR). In some embodiments, the PCR is real-time PCR.

In some of any of such embodiments, the expression level of NF-kB and/or calcineurin is decreased at the one or more time points following initiation of treatment compared to the baseline expression level.

Also provided herein is a method of determining the efficacy of a therapeutic agent for the treatment of Parkinson's Disease (PD), the method comprising: a) determining a baseline activation level of one or more canonical pathways in a biological sample from a subject having PD, wherein the one or more canonical pathways are associated with cellular immune response signaling, neuroinflammation signaling, or PD signaling pathways; b) determining an activation level of the one or more canonical pathways in a biological sample from the subject at one or more time points following initiation of treatment with a therapeutic agent; and c) comparing the activation level of the one or more canonical pathways at the one or more time points following initiation of treatment to the baseline expression level, wherein an increase in the activation of the one or more canonical pathways at the one or more time points following initiation of treatment to the baseline expression level indicates an increase in likelihood of therapeutic efficacy for the treatment of PD and/or indicates an increase in degree of effectiveness at treating PD in the subject.

In some embodiments, the comparing the activation level of the one or more canonical pathways at the one or more time points following initiation of treatment to the baseline activation level indicates a likelihood of therapeutic efficacy for the treatment of PD and/or indicates a degree of effectiveness at treating PD in the subject.

In some of any of such embodiments, if the activation level of the one or more canonical pathways at the one or more time points following initiation of treatment to the baseline activation level increased, the comparison indicates an increased likelihood of therapeutic efficacy for the treatment of PD in the subject and/or indicates an increased degree of effectiveness at treating PD in the subject. In some of any of such embodiments, an increased activation level of the one or more canonical pathways at the one or more time points following initiation of treatment as compared to the baseline activation level indicates an increase in likelihood of therapeutic efficacy for the treatment of PD in the subject and/or indicates an increase in degree of effectiveness at treating PD in the subject. In some of any of such embodiments, the activation level of the one or more canonical pathways at the one or more time points following initiation of treatment as compared to the baseline activation level increased.

In some of any of such embodiments, the activation level of at least a threshold percentage of the one or more canonical pathways have increased in the biological sample at the one or more time points following initiation of the therapy compared to the baseline activation level. In some embodiments, the threshold percentage is at least 50%, at least 55%, at least 60%, at least 65%, at least 70%, at least 75%, at least 80%, at least 85%, at least 90%, or at least 95%.

In some of any of such embodiments, the comparison indicates an increase in likelihood of therapeutic efficacy for the treatment of PD in the subject and/or indicates an increase in degree of effectiveness at treating PD in the subject if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.

Also provided herein is a method of monitoring the progression of Parkinson's Disease (PD) during treatment, the method comprising: a) determining a baseline expression level of NF-kB and/or calcineurin in a biological sample from a subject having PD prior to initiation of a treatment comprising a therapeutic agent; b) determining an expression level of NF-kB and/or calcineurin in a biological sample from a subject at one or more time points following initiation of the treatment; and c) determining if the expression level of NF-kB and/or calcineurin at the one or more time points following initiation of the treatment decreased compared to the baseline expression level.

Also provided herein is a method of monitoring the progression of Parkinson's Disease (PD) during treatment, the method comprising: a) determining a baseline expression level of NF-kB and/or calcineurin in a biological sample from a subject having PD prior to initiation of a treatment comprising a therapeutic agent; b) determining an expression level of NF-kB and/or calcineurin in a biological sample from a subject at one or more time points following initiation of the treatment; and c) comparing the expression level of NF-kB and/or calcineurin at the one or more time points following initiation of the treatment compared to the baseline expression level, wherein the comparison indicates a likelihood of slowing, reversing, and/or halting the progression of PD in the subject.

In some embodiments, the comparison indicates that the expression level of NF-kB and/or calcineurin at the one or more time points following initiation of treatment decreased compared to the baseline expression level.

In some embodiments, if the expression level of NF-kB and/or calcineurin at the one or more time points following initiation of treatment decreased compared to the baseline expression level, the comparison indicates an increased likelihood of slowing, reversing, and/or halting the progression of PD in the subject. In some embodiments, if the expression level of NF-kB and/or calcineurin at the one or more time points following initiation of treatment did not decrease compared to the baseline expression level, the comparison indicates a neutral or decreased likelihood of slowing, reversing, and/or halting the progression of PD in the subject.

In some of any of such embodiments, a decreased expression level of NF-kB and/or calcineurin at the one or more time points following initiation of treatment compared to the baseline expression level indicates an increased likelihood of slowing, reversing, and/or halting the progression of PD in the subject.

In some of any of such embodiments, if the expression level of NF-kB and/or calcineurin at the one or more time points following initiation of treatment decreased compared to the baseline expression level, the therapeutic agent is indicated as being effective at slowing, reversing, and/or halting the progression of PD in the subject. In some of any of such embodiments, a decreased expression level of NF-kB and/or calcineurin at the one or more time points following initiation of treatment compared to the baseline expression level indicates an increase in likelihood of slowing, reversing, and/or halting the progression of PD in the subject and/or indicates an increase in degree of effectiveness at slowing, reversing, and/or halting the progression of PD in the subject.

In some of any of such embodiments, the baseline expression level is determined at or at least 1 day, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment.

In some of any of such embodiments, the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment. In some of any of such embodiments, the one or more time points following initiation of treatment with the therapeutic agent comprises a time point at or about 2 months following initiation of treatment.

In some of any of such embodiments, the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.

In some of any of such embodiments, the expression level of NF-kB and/or calcineurin is determined by an assay. In some embodiments, the assay is selected from the group consisting of mass spectrometry, enzyme-linked immunosorbent assay (ELISA), western blotting, or polymerase chain reaction (PCR). In some embodiments, the PCR is real-time PCR.

In some of any of such embodiments, the expression level of NF-kB and/or calcineurin is decreased at the one or more time points following initiation of treatment compared to the baseline expression level.

Also provided herein is a method of monitoring the progression of Parkinson's Disease (PD) during treatment, the method comprising: a) determining a baseline activation level of one or more canonical pathways in a biological sample from a subject having PD prior to initiation of a treatment comprising a therapeutic agent, wherein the one or more canonical pathways are associated with cellular immune response signaling, neuroinflammation signaling, or PD signaling pathways; b) determining an activation level of the one or more canonical pathways in a biological sample from a subject at one or more time points following initiation of the treatment; and c) comparing the activation level of the one or more canonical pathways at the one or more time points following initiation of treatment to the baseline expression level, wherein an increase in the activation of the one or more canonical pathways at the one or more time points following initiation of treatment compared to the baseline expression level indicates an increase in likelihood of slowing, reversing, and/or halting the progression of PD.

In some embodiments, the comparing the activation level of the one or more canonical pathways at the one or more time points following initiation of treatment to the baseline activation level indicates a likelihood of slowing, reversing, and/or halting the progression of PD in the subject. In some of any of such embodiments, if the activation level of the one or more canonical pathways at the one or more time points following initiation of treatment to the baseline activation level increased, the comparison indicates an increased likelihood of slowing, reversing, and/or halting the progression of PD in the subject.

In some of any of such embodiments, an increased activation level of the one or more canonical pathways at the one or more time points following initiation of treatment as compared to the baseline activation level indicates an increase in likelihood of slowing, reversing, and/or halting the progression of PD in the subject.

In some of any of such embodiments, the activation level of the one or more canonical pathways at the one or more time points following initiation of treatment as compared to the baseline activation level increased.

In some of any of such embodiments, the activation level of at least a threshold percentage of the one or more canonical pathways have increased in the biological sample at the one or more time points following initiation of the therapy compared to the baseline activation level. In some embodiments, the threshold percentage is at least 50%, at least 55%, at least 60%, at least 65%, at least 70%, at least 75%, at least 80%, at least 85%, at least 90%, or at least 95%.

In some of any of such embodiments, the comparison indicates an increase in likelihood of slowing, reversing, and/or halting the progression of PD in the subject if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.

Also provided herein is a method for treating Parkinson's Disease (PD) in a subject, the method comprising: a) determining a baseline expression level of NF-kB and/or calcineurin in a biological sample from a subject having PD; b) administering a therapeutic agent for the treatment of PD to the subject; c) determining an expression level of NF-kB and/or calcineurin in a biological sample from the subject at one or more time points following initiation of the treatment; and d) determining if the expression level of NF-kB and/or calcineurin at the one or more time points following initiation of the treatment decreased compared to the baseline expression level, wherein a decrease in the expression level of NF-kB and/or calcineurin at the one or more time points following initiation of the treatment decreased compared to the baseline expression level indicates an increased likelihood of therapeutic efficacy in treating PD in the subject.

Also provided herein is a method for treating Parkinson's Disease (PD) in a subject, the method comprising: a) determining a baseline expression level of NF-kB and/or calcineurin in a biological sample from a subject having PD; b) administering a therapeutic agent for the treatment of PD to the subject; c) determining an expression level of NF-kB and/or calcineurin in a biological sample from the subject at one or more time points following initiation of the treatment; and d) comparing the expression level of NF-kB and/or calcineurin at the one or more time points following initiation of the treatment to the baseline expression level, wherein the comparison indicates a likelihood of therapeutic efficacy in treating PD in the subject.

In some embodiments, the comparison indicates that the expression level of NF-kB and/or calcineurin at the one or more time points following initiation of treatment decreased compared to the baseline expression level.

In some embodiments, if the expression level of NF-kB and/or calcineurin at the one or more time points following initiation of treatment decreased compared to the baseline expression level, the comparison indicates an increased likelihood of therapeutic efficacy for the treatment of PD in the subject and/or indicates an increased degree of effectiveness at treating PD in the subject. In some embodiments, if the expression level of NF-kB and/or calcineurin at the one or more time points following initiation of treatment did not decrease compared to the baseline expression level, the comparison indicates a neutral or decreased likelihood of therapeutic efficacy for the treatment of PD in the subject and/or indicates a neutral or decreased degree of effectiveness at treating PD in the subject.

In some of any of such embodiments, a decreased expression level of NF-kB and/or calcineurin at the one or more time points following initiation of the treatment compared to the baseline expression level indicates an increase in likelihood of therapeutic efficacy in treating PD in the subject.

In some of any of such embodiments, the baseline expression level is determined at or at least 1 day, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment.

In some of any of such embodiments, the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment. In some of any of such embodiments, the one or more time points following initiation of treatment with the therapeutic agent comprises a time point at or about 2 months following initiation of treatment.

In some of any of such embodiments, the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.

In some of any of such embodiments, the expression level of NF-kB and/or calcineurin is determined by an assay. In some embodiments, the assay is selected from the group consisting of mass spectrometry, enzyme-linked immunosorbent assay (ELISA), western blotting, or polymerase chain reaction (PCR). In some embodiments, the PCR is real-time PCR.

In some of any of such embodiments, the expression level of NF-kB and/or calcineurin is decreased at the one or more time points following initiation of treatment compared to the baseline expression level.

In some of any of such embodiments, the method further comprises continuing treatment if the comparison indicates an increased likelihood of therapeutic efficacy.

In some of any of such embodiments, the method further comprises continuing treatment if the expression level of NF-kB and/or calcineurin at the one or more time points following initiation of the treatment decreased compared to the baseline expression level.

In some of any of such embodiments, the method further comprises discontinuing treatment if the expression level of NF-kB and/or calcineurin at the one or more time points following initiation of the treatment did not decrease compared to the baseline expression level.

In some of any of such embodiments: (i) treatment is continued if the expression level of NF-kB and/or calcineurin have decreased in the biological sample at the one or more time points following initiation of the therapy compared to the baseline expression level; or (ii) treatment is discontinued or altered if the expression level of NF-kB and/or calcineurin have not decreased in the biological sample at the one or more time points following initiation of the therapy compared to the baseline expression level.

In some of any of such embodiments, determining the baseline expression level of NF-kB and/or calcineurin occurs prior to administering the therapy.

Also provided herein is a method for treating Parkinson's Disease (PD) in a subject, the method comprising: a) determining a baseline activation level of one or more canonical pathways in a biological sample from a subject having PD, wherein the one or more canonical pathways are associated with cellular immune response signaling, neuroinflammation signaling, or PD signaling pathways; b) administering a therapeutic agent for the treatment of PD to the subject; c) determining an activation level of one or more canonical pathways in a biological sample from the subject at one or more time points following initiation of the treatment; and d) comparing the activation level of the one or more canonical pathways at the one or more time points following initiation of treatment to the baseline expression level, wherein an increase in the activation level of the one or more canonical pathways indicates an increased likelihood of therapeutic efficacy.

In some embodiments, the method further comprises continuing treatment if the activation level of the one or more canonical pathways indicates an increased likelihood of therapeutic efficacy.

In some of any of such embodiments: (i) treatment is continued if the activation level of the one or more canonical pathways have increased in the biological sample at the one or more time points following initiation of the therapy compared to the baseline activation level; or (ii) treatment is discontinued or altered if the activation level of the one or more canonical pathways have not increased in the biological sample at the one or more time points following initiation of the therapy compared to the baseline activation level. In some of any of such embodiments, treatment is continued if the activation level of at least a threshold percentage of the one or more canonical pathways have increased in the biological sample at the one or more time points following initiation of the therapy compared to the baseline activation level.

In some of any of such embodiments, the activation level of at least a threshold percentage of the one or more canonical pathways have increased in the biological sample at the one or more time points following initiation of the therapy compared to the baseline activation level.

In some of any of such embodiments, the threshold percentage is at least 50%, at least 55%, at least 60%, at least 65%, at least 70%, at least 75%, at least 80%, at least 85%, at least 90%, or at least 95%. In some of any of such embodiments, treatment is continued if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level. In some of any of such embodiments, the activation level of the one or more canonical pathways indicates an increased likelihood of therapeutic efficacy if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.

In some of any of such embodiments, the one or more canonical pathways comprises a pathway associated with cellular immune response signaling, neuroinflammation signaling, and/or PD signaling, or any combination thereof.

In some of any of such embodiments, the one or more canonical pathways are selected from the group consisting of IL-8 Signaling, fMLP Signaling in Neutrophils, Role of NFAT in Regulation of the Immune Response, Fcγ Receptor-mediated Phagocytosis in Macrophages and Monocytes, CXCR4 Signaling, PKCθ Signaling in T Lymphocytes, Leukocyte Extravasation Signaling, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, CCR3 Signaling in Eosinophils, IL-3 Signaling, GM-CSF Signaling, Macropinocytosis Signaling, IL-7 Signaling Pathway, Interferon Signaling, Nur77 Signaling in T Lymphocytes, IL-9 Signaling, Antiproliferative Role of TOB in T Cell Signaling, CCR5 Signaling in Macrophages, Role of PKR in Interferon Induction and Antiviral Response, Production of Nitric Oxide and Reactive Oxygen Species in Macrophages, and iNOS Signaling.

In some of any of such embodiments, the one or more canonical pathways are selected from the group consisting of fMLP Signaling in Neutrophils, Role of NFAT in Regulation of the Immune Response, PKCθ Signaling in T Lymphocytes, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, IL-3 Signaling, GM-CSF Signaling, Nur77 Signaling in T Lymphocytes, IL-9 Signaling, Role of PKR in Interferon Induction and Antiviral Response, Production of Nitric Oxide and Reactive Oxygen Species in Macrophages, and iNOS Signaling.

In some of any of such embodiments, the therapeutic agent is selected from the group consisting of granulocyte macrophage colony stimulating factor (GM-CSF) or an analog thereof, GM-CSF mRNA, vasoactive intestinal peptide (VIP) or an analog thereof, and VIP mRNA.

In some of any of such embodiments, the therapeutic agent is an agonist of granulocyte-macrophage colony stimulating factor 2 receptor (CSF2R) or vasoactive intestinal peptide receptor 2 (VIP2R). In some of any of such embodiments, the agonist is a peptide or peptide-like agonist of CSF2R or VIP2R. In some of any of such embodiments, the agonist is an antibody or a fragment thereof that binds to CSF2R or VIP2R.

In some of any of such embodiments, the therapeutic agent is a mimetic of GM-CSF or is a mimetic of VIP.

In some of any of such embodiments, the therapeutic agent is levodopa.

In some of any of such embodiments, the therapeutic agent is a dopamine receptor agonist.

In some of any of such embodiments, the GM-CSF or analog thereof is sargramostim. In some of any of such embodiments, the sargramostim is administered subcutaneously five times per week. In some of any of such embodiments, the GM-CSF or analog thereof is molgramostim. In some of any of such embodiments, the VIP or analog thereof is aviptadil. In some of any of such embodiments, the GM-CSF mRNA encodes sargramostim or molgramostim. In some of any of such embodiments, the VIP mRNA encodes aviptadil.

In some of any of such embodiments, the therapeutic agent is an agent that reacts with and/or induces molecules that react with a GM-CSF receptor or a VIP receptor. In some of any of such embodiments, the GM-CSF receptor is CSF2R and/or the VIP receptor is VIP2R.

In some of any of such embodiments, the therapeutic agent is an immunogen that induces a humoral immune response against at least one abnormal protein of PD. In some of any of such embodiments, the immunogen is nitrated alpha synuclein or a fragment thereof. In some of any of such embodiments, the immunogen is a nitrated alpha synuclein fragment, and wherein the nitrated alpha synuclein fragment is a carboxy terminal fragment consisting of the carboxy terminal 20 amino acids of alpha synuclein up to the carboxy terminal 70 amino acids of alpha synuclein. In some of any of such embodiments, the immunogen is human nitrated alpha synuclein or a fragment thereof. In some of any of such embodiments, the immunogen is comprised in a composition that further comprises at least one adjuvant that stimulates regulatory T cells. In some of any of such embodiments, the adjuvant is selected from the group consisting of VIP, vitamin D, GM-CSF, and transforming growth factor beta (TGFβ). In some of any of such embodiments, the nitrated alpha synuclein comprises the amino acid sequence of SEQ ID NO: 4, or comprises an amino acid sequence having at least 90%, 91%, 92%, 93%, 94%, 95%, 96%, 97%, 98%, or 99% sequence identity to the amino acid sequence of SEQ ID NO: 4. In some of any of such embodiments, the nitrated alpha synuclein fragment comprises amino acid residues 101-140 of SEQ ID NO: 4, or comprises an amino acid sequence having at least 90%, 91%, 92%, 93%, 94%, 95%, 96%, 97%, 98%, or 99% sequence identity to amino acid residues 101-140 of the amino acid sequence of SEQ ID NO: 4.

In some of any of such embodiments, the composition further comprises a pharmaceutically acceptable carrier.

In some of any of such embodiments, the biological sample comprises peripheral blood lymphocytes. In some of any of such embodiments, the biological sample comprises T cells. In some of any of such embodiments, the biological sample is obtained from the subject by leukapheresis.

Also provided herein is a composition for detecting NF-kB and/or calcineurin expression in a biological sample, wherein the composition comprises a kit for a mass spectrometry analysis, an ELISA assay, a western blotting assay, or a PCR reaction.

In some embodiments of the composition, the kit comprises a reagent for an ELISA assay. In some embodiments, the kit for the ELISA assay comprises a multi-well sample plate that is coated with immobilized capture antibodies that bind to NF-kB and/or calcineurin; detection antibodies covalently linked to an enzyme wherein the detection antibodies also bind to NF-kB and/or calcineurin; a colored or fluorescent product that will be catalyzed by the enzyme attached to the detection antibody; and appropriate buffers.

Also provided herein are biomarkers for monitoring the progression of Parkinson's Disease (PD) and/or the effectiveness of therapeutics for the treatment of PD, wherein the biomarkers are NF-kB and calcineurin; or are one or more canonical pathways associated with cellular immune response signaling, neuroinflammation signaling, or PD signaling pathways.

In some embodiments, the one or more canonical pathways are selected from the group consisting of IL-8 Signaling, fMLP Signaling in Neutrophils, Role of NFAT in Regulation of the Immune Response, Fcγ Receptor-mediated Phagocytosis in Macrophages and Monocytes, CXCR4 Signaling, PKCθ Signaling in T Lymphocytes, Leukocyte Extravasation Signaling, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, CCR3 Signaling in Eosinophils, IL-3 Signaling, GM-CSF Signaling, Macropinocytosis Signaling, IL-7 Signaling Pathway, Interferon Signaling, Nur77 Signaling in T Lymphocytes, IL-9 Signaling, Antiproliferative Role of TOB in T Cell Signaling, CCR5 Signaling in Macrophages, Role of PKR in Interferon Induction and Antiviral Response, Production of Nitric Oxide and Reactive Oxygen Species in Macrophages, and iNOS Signaling.

In some of any of such embodiments, the one or more canonical pathways are selected from the group consisting of fMLP Signaling in Neutrophils, Role of NFAT in Regulation of the Immune Response, PKCθ Signaling in T Lymphocytes, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, IL-3 Signaling, GM-CSF Signaling, Nur77 Signaling in T Lymphocytes, IL-9 Signaling, Role of PKR in Interferon Induction and Antiviral Response, Production of Nitric Oxide and Reactive Oxygen Species in Macrophages, and iNOS Signaling.

Also provided herein is a therapeutic agent for use in the treatment of Parkinson's Disease (PD) in a subject, wherein the therapeutic agent is for use in a method comprising: a) determining a baseline expression level of NF-kB and/or calcineurin in a biological sample from a subject having PD; b) determining an expression level of NF-kB and/or calcineurin in a biological sample from the subject at one or more time points following administration of the treatment; and c) comparing the expression level of NF-kB and/or calcineurin at the one or more time points following initiation of the treatment to the baseline expression level, wherein the comparison indicates a likelihood of therapeutic efficacy in treating PD in the subject.

Also provided herein is the use of a therapeutic agent in the treatment of Parkinson's Disease (PD) in a subject, wherein the use comprises: a) determining a baseline expression level of NF-kB and/or calcineurin in a biological sample from a subject having PD; b) determining an expression level of NF-kB and/or calcineurin in a biological sample from the subject at one or more time points following administration of the therapeutic agent; and c) comparing the expression level of NF-kB and/or calcineurin at the one or more time points following initiation of the treatment to the baseline expression level, wherein the comparison indicates a likelihood of therapeutic efficacy in treating PD in the subject.

Also provided herein is a therapeutic agent for use in the treatment of Parkinson's Disease (PD) in a subject, wherein the therapeutic agent is for use in a method comprising: a) determining a baseline activation level of one or more canonical pathways in a biological sample from a subject having PD, wherein the one or more canonical pathways are associated with cellular immune response signaling, neuroinflammation signaling, or PD signaling pathways; b) determining an activation level of one or more canonical pathways in a biological sample from the subject at one or more time points following administration of the treatment; and c) comparing the activation level of one or more canonical pathways in a biological sample from the subject at the one or more time points following initiation of the treatment to the baseline expression level, wherein the comparison indicates a likelihood of therapeutic efficacy in treating PD in the subject.

Also provided herein is the use of a therapeutic agent in the treatment of Parkinson's Disease (PD) in a subject, wherein the use comprises: a) determining a baseline activation level of one or more canonical pathways in a biological sample from a subject having PD, wherein the one or more canonical pathways are associated with cellular immune response signaling, neuroinflammation signaling, or PD signaling pathways; b) determining an activation level of one or more canonical pathways in a biological sample from the subject at one or more time points following administration of the treatment; and c) comparing the activation level of one or more canonical pathways in the biological sample from the subject at the one or more time points following initiation of the treatment to the baseline expression level, wherein the comparison indicates a likelihood of therapeutic efficacy in treating PD in the subject.

### BRIEF DESCRIPTION OF THE DRAWINGS

FIG. 1A depicts the gating strategy for CD4+ and Treg populations. FIG. 1B shows T responder cells (Tresp) cells at baseline and at 2, 4, 6, 8, 10, and 12 months of Leukine treatment where they are assessed for their ability to suppress Tresp proliferation. FIG. 1C shows representative schematic for assessing area under the curve (AUC) to determine Treg activity for sargramostim treatment vs baseline treatment. Dotted lines represent the number of Treg needed for 50% inhibition at baseline (darker shading) and with sargramostim treatment (lighter shading).

FIG. 2A-E shows FMO gating strategy for populations within peripheral blood.

FIG. 3A-D shows adverse events (AE) comparing two clinical trials of sargramostim. PD subjects were administered sargramostim (Leukine®, human recombinant GM-CSF) in a previous Phase 1a (Ph 1a) (n=10) and current Phase 1b (Ph 1b) clinical trial (n=5), with Phase 1a (1a) on the left and Phase 1b (1b) on the right for Total and Total/month (Mo). FIG. 3C shows a plot for adverse events in Phase 1a, and FIG. 3D shows a plot for adverse events in Phase 1b.

FIG. 4A-F shows Unified Parkinson's Disease Rating Scale (UPDRS) Part III motor assessment before and during sargramostim treatment. FIG. 4A shows raw UPDRS Part III scores over time grouped for individual subjects. FIG. 4B shows the total mean UPDRS Part III scores grouped by time of treatment. The dashed line indicates mean baseline measurement. FIG. 4C shows mean UPDRS Part III scores±SD grouped by combined (All) and individual subjects. Specific p values are indicated above each subject. FIG. 4D shows change from baseline in UPDRS Part III scores over time grouped for individual subjects. FIG. 4E shows mean change from baseline±SD in UPDRS Part III scores grouped by time of treatment. FIG. 4F shows mean change from baseline in UPDRS Part III scores±SD grouped by combined (All) and individual subjects. Specific p values are indicated above each subject.

FIG. 5A-I shows flow cytometric analysis of CD4+ peripheral blood populations over time.

FIG. 6A-E shows flow cytometric analysis, immunosuppressive function, and FOXP3 Treg-Specific Demethylated Region (TSDR) methylation status of CD4+CD25+CD127low Treg subsets within CD4+ peripheral blood lymphocytes. FIG. 6F shows percent demethylation (±SD) within the TSDR of the FOXP3 intron from isolated Tregs before and at 2 and 6 months after initiation of sargramostim treatment. FIG. 6G shows quantification of Treg-mediated suppression (±SD) of Tresp (CD4+CD25−) proliferation at various Tresp:Treg ratios. Treg-mediated suppression is calculated as % Inhibition=1−(% Proliferating Tresp:Treg÷% Proliferating Stimulated Tresp Alone) and is reported as percent inhibition. FIG. 6H shows correlation analysis for percent TSDR demethylation and corresponding Treg-mediated inhibition (Treg activity, AUC) at baseline, 2-months, and 6-months during sargramostim treatment, indicating a direct correlation of TSDR demethylation and Treg activity.

FIG. 7A-M shows flow cytometric analysis of CD4+ peripheral blood populations comparing baseline and pooled sargramostim treatment for combined and individual subjects.

FIG. 8A-E shows flow cytometric analysis of CD4+CD25+CD127high Teff within peripheral blood over time.

FIG. 9A-M shows flow cytometric analysis of CD4+CD25+CD127high Teff markers comparing baseline and sargramostim treatment for combined or individual subjects.

FIG. 10A-J shows flow cytometric analysis of CD4+CD25+CD127low Treg markers comparing baseline and sargramostim treatment for all subjects combined and individual subjects.

FIG. 11A-H shows elevated peripheral blood markers are associated with enhanced Treg function.

FIG. 12A-E shows elevated peripheral blood markers and enhanced Treg function are associated with decreased UPDRS Part III scores.

FIG. 13A-G shows correlation analyses of peripheral blood markers with Treg number necessary for 50% inhibitory activity.

FIG. 14A-B shows transcriptomic analysis of innate and adaptive immune response gene regulation within peripheral blood lymphocytes isolated from subjects at baseline and following sargramostim treatment.

FIG. 14A shows a heat map depicting gene regulation in lymphocytes isolated from subjects after 2 months (2 mo) and 6 months (6 mo) of sargramostim treatment compared to baseline values for individual subjects (2001, 2003, and 2005).

FIG. 14B shows tables of significantly dysregulated genes within peripheral blood lymphocyte populations for individual subjects.

FIG. 15 shows differential proteomic analysis of peripheral blood lymphocytes at 2 and 6 months after treatment. Volcano plots showing the fold change (treatment versus baseline) plotted against the p value highlighting significantly changed proteins.

### DETAILED DESCRIPTION

Parkinson's disease (PD) is the most common neurodegenerative motor disorder heralded by reductions in striatal dopamine and numbers of dopaminergic neurons in the substantia nigra pars compacta (Schwab A D et al., Neurobiol Dis 2020; 137: 104760). While palliative therapies abound, clinical trials designed for efficacy of disease-modifying strategies have largely failed, suggesting that either hypotheses are limited or limitations are inherent in study design, implementation, or clinical outcome assessment (Olanow C W et al., Ann Neurol 2008; 64 Suppl 2: S101-10).

The linkages between clinical and disease biology may parallel the heterogeneity of diverse PD pathobiology. Of the suspected etiologies of PD, neuroinflammation and peripheral immune dysfunction stand concordant (Schwab A D et al., Neurobiol Dis 2020; 137: 104760; Guzman-Martinez L et al., Front Pharmacol 2019; 10: 1008; Machhi J et al. Mol Neurodegener 2020; 15(1): 32). Targeting immune response components can potentially mitigate disease. This is achieved by balancing numbers and function of regulatory and effector T cells (Treg and Teff) in the periphery and along the nigrostriatal axis (Machhi J et al., Mol Neurodegener 2020; 15(1): 32).

It has been demonstrated that Tregs attenuate neuroinflammation and protect dopaminergic neurons from injury and loss (Machhi J et al., Mol Neurodegener 2020; 15(1): 32; Kosloski L M et al., J Neuroimmunol 2013; 265(1-2): 1-10; Olson K E et al., J Neurosci 2015; 35(50): 16463-78; Reynolds A D, et al., J Leukoc Biol 2007; 82(5): 1083-94). PD patient Tregs are impaired in their immunosuppressive activities, and Teff subsets with neurotoxic potential are present during disease (Saunders J A et al. J Neuroimmune Pharmacol 2012; 7(4): 927-38; Chao Y et al., Biomed Res Int 2014; 2014: 308654; Lindestam Arlehamn C S et al., Nat Commun 2020; 11(1): 1875; Kustrimovic N et al., J Neuroinflammation 2018; 15(1): 205). Studies indicate that increased Teff phenotypes are associated with worsening of UPDRS Part III scores, while others suggest that α-synuclein reactive T cells are elevated in early disease but wane over time (Saunders J A et al., J Neuroimmune Pharmacol 2012; 7(4): 927-38; Lindestam Arlehamn C S et al. Nat Commun 2020; 11(1): 1875). Nonetheless, the presence of autoreactive T cells and elevated proinflammatory responses is confirmed, but the exact association and time-course with respect to disease progression is still being explored. Potentiation of Treg function and modulation of Teff responses restores adaptive immune regulation and represents a means to harness neurodegeneration (Machhi J et al., Mol Neurodegener 2020; 15(1): 32). Studies have demonstrated that granulocyte-macrophage colony-stimulating factor (GM-CSF, sargramostim, Leukine) increases Treg numbers and function and protects dopaminergic neurons (Kosloski L M et al., J Neuroimmunol 2013; 265(1-2): 1-10; Olson K E et al., Neurotherapeutics 2020; Gendelman H E et al., NPJ Parkinsons Dis 2017; 3: 10).

Translation to humans validated GM-CSF activities in a double-blind, placebo-controlled Phase 1 PD clinical trial (Gendelman H E et al., NPJ Parkinsons Dis 2017; 3: 10). Daily administration of high-dose sargramostim (6 μg/kg/day) increased Treg numbers and function with improved UPDRS-scored motor function and magnetoencephalography assessed neurophysiological activities. Although treatment was generally well-tolerated, sargramostim led to select adverse events including injection site reactions, bone pain, and immune reactions including urticaria and vasculitis.

The present disclosure, in some aspects, relates to a novel dosing regimen in which the dosing regimen is lowered and the time of the study evaluation is extended, with the safety and tolerability of a year-long reduced dose of a drug, such as sargramostim, as part of a treatment regimen being evaluated in the treatment of PD. The present disclosure, in some aspects, also relates to monitoring, assessing, and determining one or more biomarkers, such as NF-κB and/or calcineurin, during treatment of PD, e.g., for monitoring the progression of PD and/or the therapeutic efficacy of a therapeutic for the treatment of PD and/or as part of a method of treating PD.

The present disclosure describes novel biomarkers for Parkinson's Diseases. Specifically, NF-kB and calcineurin have been identified as biomarkers whose expression changes in response to specific treatments for Parkinson's Disease and can be used to track the progression of the diseases and be used as a readout for the effectiveness of various treatments.

In the present disclosure, the biomarkers include NF-kB, calcineurin, NF-kB pathway members, calcineurin pathway members or combinations thereof. In some embodiments, a decrease in the levels of one or both of these biomarkers corresponds to a beneficial transformation of the immune profile and the immune microenvironment. In some embodiments, a decrease in the levels of one or both of these biomarkers corresponds to a beneficial decrease in inflammation. The biomarkers of the present disclosure may also be used in combination with other Parkinson's Disease biomarkers to help monitor disease progression and response to therapy. In some embodiments, the biomarkers of the present disclosure are used in combination with the biomarkers described in US20140349877 (incorporated by reference herein) and US20190117735 (incorporated by reference herein).

In the present disclosure, the biomarkers can be used as a marker or readout for the effectiveness of existing or novel therapeutics for the treatment of Parkinson's Disease. In some embodiments, a therapeutic for the treatment of Parkinson's Disease may include but is not limited to small molecules, antibodies, antibody fragments, antibody drug conjugates, peptides, proteins, cell therapies, and nucleic acid-based therapies (such as miRNA, mRNA, siRNA). In some embodiments, the therapeutic is a drug capable of modulating the immune system including but not limited to GM-CSF (Leukine, Sargramostim), GM-CSF analogs, GM-CSF mRNA, VIP, VIP analogs, vaccines, immunogens, etc. In some embodiments, one or both of the biomarker's expression levels will decrease during treatment with a therapeutic. In some embodiments, the decrease in the expression of one or both of the biomarkers in response to treatment with a therapeutic is an indication that the therapeutic is having a beneficial effect in the subject.

The biomarkers of the present disclosure may be detected in a variety of biological samples obtained from a subject including but not limited to blood, plasma, and cerebral spinal fluid. In some embodiments, the biomarkers of the present disclosure may be detected in a biological sample using a variety of assay techniques including, mass spectrometry, ELISA, western blotting, and PCR.

Compositions of the present disclosure also include kits necessary for measuring the biomarkers in a biological sample. In some embodiments, the composition consists of a kit that includes a multi-well sample plate that is coated with immobilized capture antibodies that bind to NF-kB and/or calcineurin; detection antibodies covalently linked to an enzyme wherein the detection antibodies also bind to NF-kB and/or calcineurin; a colored or fluorescent product that will be catalyzed by the enzyme attached to the detection antibody; and appropriate buffers. In some embodiments, the ELISA plate will be specific for detecting one of the biomarkers. In another embodiment, the ELISA plate will be able to detect both biomarkers.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Biomarkers for Parkinson's Disease (PD)

Provided herein are methods, compositions, and kits involving biomarkers for Parkinson's Disease (PD).

### A. Methods Involving Biomarkers for Parkinson's Disease

Provided herein are methods that include determining the expression level of NF-kB and/or calcineurin in a biological sample from a subject having Parkinson's Disease (PD), prior to and following initiation of treatment with a therapeutic agent; and/or include determining the activation level of one or more canonical pathways, e.g., one or more canonical pathways associated with cellular immune response signaling, neuroinflammation signaling, or PD signaling pathways, prior to and following initiation of treatment with a therapeutic agent. In some embodiments, the methods provided herein allow for determining the efficacy of a therapeutic agent for the treatment of PD. In some embodiments, the methods provided herein allow for monitoring the progression of Parkinson's Disease (PD) during treatment. In some embodiments, the methods provided herein allow for the treatment of PD by indicating whether treatment is to be continued or discontinued or altered. In some embodiments, altering the treatment comprises administering a different treatment, e.g., a different therapeutic agent, and/or comprises increasing or decreasing the dosing and/or frequency of the therapeutic agent.

### 1. Methods of Determining the Efficacy of a Therapeutic Agent for the Treatment of Parkinson's Disease

Provided herein are methods of determining the efficacy of a therapeutic agent for the treatment of Parkinson's Disease (PD), the method comprising: a) determining a baseline expression level of NF-kB and/or calcineurin in a biological sample from a subject having PD; b) determining an expression level of NF-kB and/or calcineurin in a biological sample from the subject at one or more time points following initiation of treatment with a therapeutic agent; and c) determining if the expression level of NF-kB and/or calcineurin at the one or more time points following initiation of treatment decreased compared to the baseline expression level.

Also provided herein are methods of determining the efficacy of a therapeutic agent for the treatment of Parkinson's Disease (PD), the method comprising: a) determining a baseline expression level of NF-kB and/or calcineurin in a biological sample from a subject having PD; b) determining an expression level of NF-kB and/or calcineurin in a biological sample from a subject at one or more time points following initiation of treatment with a therapeutic agent; and c) comparing the expression level of NF-kB and/or calcineurin at the one or more time points following initiation of treatment to the baseline expression level, wherein the comparison indicates a likelihood of therapeutic efficacy for the treatment of PD in the subject and/or indicates a degree of effectiveness at treating PD in the subject.

In some embodiments, the comparison indicates that the expression level of NF-kB and/or calcineurin at the one or more time points following initiation of treatment decreased compared to the baseline expression level. In some embodiments, the comparison indicates that the expression level of NF-kB at the one or more time points following initiation of treatment decreased compared to the baseline expression level. In some embodiments, the comparison indicates that the expression level of calcineurin at the one or more time points following initiation of treatment decreased compared to the baseline expression level. In some embodiments, the comparison indicates that the expression level of NF-kB and calcineurin at the one or more time points following initiation of treatment decreased compared to the baseline expression level.

In some embodiments, if the expression level of NF-kB and/or calcineurin at the one or more time points following initiation of treatment decreased compared to the baseline expression level, the comparison indicates an increased likelihood of therapeutic efficacy for the treatment of PD in the subject and/or indicates an increased degree of effectiveness at treating PD in the subject.

In some embodiments, if the expression level of NF-kB and/or calcineurin at the one or more time points following initiation of treatment did not decrease compared to the baseline expression level, the comparison indicates a neutral or decreased likelihood of therapeutic efficacy for the treatment of PD in the subject and/or indicates a neutral or decreased degree of effectiveness at treating PD in the subject.

In some embodiments, a decreased expression level of NF-kB and/or calcineurin at the one or more time points following initiation of treatment compared to the baseline expression level indicates an increase in likelihood of therapeutic efficacy for the treatment of PD in the subject and/or indicates an increase in degree of effectiveness at treating PD in the subject. In some embodiments, a decreased expression level of NF-kB at the one or more time points following initiation of treatment compared to the baseline expression level indicates an increase in likelihood of therapeutic efficacy for the treatment of PD in the subject and/or indicates an increase in degree of effectiveness at treating PD in the subject. In some embodiments, a decreased expression level of calcineurin at the one or more time points following initiation of treatment compared to the baseline expression level indicates an increase in likelihood of therapeutic efficacy for the treatment of PD in the subject and/or indicates an increase in degree of effectiveness at treating PD in the subject. In some embodiments, a decreased expression level of NF-kB and calcineurin at the one or more time points following initiation of treatment compared to the baseline expression level indicates an increase in likelihood of therapeutic efficacy for the treatment of PD in the subject and/or indicates an increase in degree of effectiveness at treating PD in the subject.

In some embodiments, the expression level of NF-kB and/or calcineurin is decreased at the one or more time points following initiation of treatment compared to the baseline expression level. In some embodiments, the expression level of NF-kB is decreased at the one or more time points following initiation of treatment compared to the baseline expression level. In some embodiments, the expression level of calcineurin is decreased at the one or more time points following initiation of treatment compared to the baseline expression level. In some embodiments, the expression level of NF-kB and calcineurin is decreased at the one or more time points following initiation of treatment compared to the baseline expression level.

Also provided herein are methods of determining the efficacy of a therapeutic agent for the treatment of Parkinson's Disease (PD), the method comprising: a) determining a baseline expression level of NF-kB and/or calcineurin in a biological sample from a subject having PD; b) determining an expression level of NF-kB and/or calcineurin in a biological sample from the subject at one or more time points following initiation of treatment with a therapeutic agent; and c) determining if the expression level of NF-kB and/or calcineurin at the one or more time points following initiation of treatment decreased compared to the baseline expression level; wherein if the expression level of NF-kB and/or calcineurin at the one or more time points following initiation of treatment decreased compared to the baseline expression level, the comparison indicates an increased likelihood of therapeutic efficacy for the treatment of PD in the subject and/or indicates an increased degree of effectiveness at treating PD in the subject.

Also provided herein are methods of determining the efficacy of a therapeutic agent for the treatment of Parkinson's Disease (PD), the method comprising: a) determining a baseline activation level of one or more canonical pathways in a biological sample from a subject having PD, wherein the one or more canonical pathways are associated with cellular immune response signaling, neuroinflammation signaling, or PD signaling pathways; b) determining an activation level of the one or more canonical pathways in a biological sample from the subject at one or more time points following initiation of treatment with a therapeutic agent; and c) comparing the activation level of the one or more canonical pathways at the one or more time points following initiation of treatment to the baseline expression level, wherein an increase in the activation of the one or more canonical pathways at the one or more time points following initiation of treatment to the baseline expression level indicates an increase in likelihood of therapeutic efficacy for the treatment of PD and/or indicates an increase in degree of effectiveness at treating PD in the subject.

In some embodiments, the comparing the activation level of the one or more canonical pathways at the one or more time points following initiation of treatment to the baseline activation level indicates a likelihood of therapeutic efficacy for the treatment of PD and/or indicates a degree of effectiveness at treating PD in the subject.

In some embodiments, if the activation level of the one or more canonical pathways at the one or more time points following initiation of treatment to the baseline activation level increased, the comparison indicates an increased likelihood of therapeutic efficacy for the treatment of PD in the subject and/or indicates an increased degree of effectiveness at treating PD in the subject.

In some embodiments, an increased activation level of the one or more canonical pathways at the one or more time points following initiation of treatment as compared to the baseline activation level indicates an increase in likelihood of therapeutic efficacy for the treatment of PD in the subject and/or indicates an increase in degree of effectiveness at treating PD in the subject.

In some embodiments, the activation level of the one or more canonical pathways at the one or more time points increased following initiation of treatment as compared to the baseline activation level.

In some embodiments, an increase in likelihood of therapeutic efficacy for the treatment of PD in the subject and/or an increase in degree of effectiveness at treating PD in the subject, is used as a measure for deciding whether to continue, discontinue, or alter the course of treatment. In some embodiments, altering treatment comprises administering a different treatment and/or increasing or decreasing the dosage and/or frequency of administration of the therapeutic agent.

In some embodiments, the increase in likelihood of therapeutic efficacy for the treatment of PD in the subject and/or the increase in degree of effectiveness at treating PD in the subject is based on the relative amount by which the expression level of NF-kB and/or calcineurin decreased compared to the baseline expression level. For instance, in some embodiments, a greater relative decrease in expression level of NF-kB and/or calcineurin results in a higher likelihood of therapeutic efficacy for the treatment of PD in the subject and/or a higher increase in degree of effectiveness at treating PD in the subject, as compared to a lesser relative decrease in expression level of NF-kB and/or calcineurin.

In some embodiments, the increase in likelihood of therapeutic efficacy for the treatment of PD in the subject and/or the increase in degree of effectiveness at treating PD in the subject is based on the relative amount by which the activation level of the one or more canonical pathways increased compared to the baseline expression level. For instance, in some embodiments, a greater relative increase in activation level of the one or more canonical pathways results in a higher likelihood of therapeutic efficacy for the treatment of PD in the subject and/or a higher increase in degree of effectiveness at treating PD in the subject, as compared to a lesser relative increase in activation level of the one or more canonical pathways.

### 2. Methods for Monitoring the Progression of Parkinson's Disease During Treatment

Provided herein are methods of monitoring the progression of Parkinson's Disease (PD) during treatment, the method comprising: a) determining a baseline expression level of NF-kB and/or calcineurin in a biological sample from a subject having PD prior to initiation of a treatment comprising a therapeutic agent; b) determining an expression level of NF-kB and/or calcineurin in a biological sample from a subject at one or more time points following initiation of the treatment; and c) determining if the expression level of NF-kB and/or calcineurin at the one or more time points following initiation of the treatment decreased compared to the baseline expression level.

Also provided herein are methods of monitoring the progression of Parkinson's Disease (PD) during treatment, the method comprising: a) determining a baseline expression level of NF-kB and/or calcineurin in a biological sample from a subject having PD prior to initiation of a treatment comprising a therapeutic agent; b) determining an expression level of NF-kB and/or calcineurin in a biological sample from a subject at one or more time points following initiation of the treatment; and c) comparing the expression level of NF-kB and/or calcineurin at the one or more time points following initiation of the treatment compared to the baseline expression level, wherein the comparison indicates a likelihood of slowing, reversing, and/or halting the progression of PD in the subject.

In some embodiments, the comparison indicates that the expression level of NF-kB and/or calcineurin at the one or more time points following initiation of treatment decreased compared to the baseline expression level.

In some embodiments, if the expression level of NF-kB and/or calcineurin at the one or more time points following initiation of treatment decreased compared to the baseline expression level, the comparison indicates an increased likelihood of slowing, reversing, and/or halting the progression of PD in the subject. In some embodiments, if the expression level of NF-kB and/or calcineurin at the one or more time points following initiation of treatment did not decrease compared to the baseline expression level, the comparison indicates a neutral or decreased likelihood of slowing, reversing, and/or halting the progression of PD in the subject.

In some embodiments, a decreased expression level of NF-kB and/or calcineurin at the one or more time points following initiation of treatment compared to the baseline expression level indicates an increased likelihood of slowing, reversing, and/or halting the progression of PD in the subject. In some embodiments, a decreased expression level of NF-kB and/or calcineurin at the one or more time points following initiation of treatment compared to the baseline expression level indicates an increased likelihood of slowing the progression of PD in the subject. In some embodiments, a decreased expression level of NF-kB and/or calcineurin at the one or more time points following initiation of treatment compared to the baseline expression level indicates an increased likelihood of reversing the progression of PD in the subject. In some embodiments, a decreased expression level of NF-kB and/or calcineurin at the one or more time points following initiation of treatment compared to the baseline expression level indicates an increased likelihood of halting the progression of PD in the subject.

In some embodiments, if the expression level of NF-kB and/or calcineurin at the one or more time points following initiation of treatment decreased compared to the baseline expression level, the therapeutic agent is indicated as being effective at slowing, reversing, and/or halting the progression of PD in the subject.

In some embodiments, a decreased expression level of NF-kB and/or calcineurin at the one or more time points following initiation of treatment compared to the baseline expression level indicates an increase in likelihood of slowing, reversing, and/or halting the progression of PD in the subject and/or indicates an increase in degree of effectiveness at slowing, reversing, and/or halting the progression of PD in the subject.

Also provided herein are methods of monitoring the progression of Parkinson's Disease (PD) during treatment, the method comprising: a) determining a baseline activation level of one or more canonical pathways in a biological sample from a subject having PD prior to initiation of a treatment comprising a therapeutic agent, wherein the one or more canonical pathways are associated with cellular immune response signaling, neuroinflammation signaling, or PD signaling pathways; b) determining an activation level of the one or more canonical pathways in a biological sample from a subject at one or more time points following initiation of the treatment; and c) comparing the activation level of the one or more canonical pathways at the one or more time points following initiation of treatment to the baseline expression level, wherein an increase in the activation of the one or more canonical pathways at the one or more time points following initiation of treatment compared to the baseline expression level indicates an increase in likelihood of slowing, reversing, and/or halting the progression of PD.

In some embodiments, the comparing the activation level of the one or more canonical pathways at the one or more time points following initiation of treatment to the baseline activation level indicates a likelihood of slowing, reversing, and/or halting the progression of PD in the subject.

In some embodiments, if the activation level of the one or more canonical pathways at the one or more time points following initiation of treatment to the baseline activation level increased, the comparison indicates an increased likelihood of slowing, reversing, and/or halting the progression of PD in the subject.

In some embodiments, an increased activation level of the one or more canonical pathways at the one or more time points following initiation of treatment as compared to the baseline activation level indicates an increase in likelihood of slowing, reversing, and/or halting the progression of PD in the subject. In some embodiments, an increased activation level of the one or more canonical pathways at the one or more time points following initiation of treatment as compared to the baseline activation level indicates an increase in likelihood of slowing the progression of PD in the subject. In some embodiments, an increased activation level of the one or more canonical pathways at the one or more time points following initiation of treatment as compared to the baseline activation level indicates an increase in likelihood of reversing the progression of PD in the subject. In some embodiments, an increased activation level of the one or more canonical pathways at the one or more time points following initiation of treatment as compared to the baseline activation level indicates an increase in likelihood of halting the progression of PD in the subject.

In some embodiments, the activation level of the one or more canonical pathways at the one or more time points following initiation of treatment as compared to the baseline activation level increased.

In some embodiments, an increase in likelihood of slowing, reversing, and/or halting the progression of PD in the subject, is used as a measure for deciding whether to continue, discontinue, or alter the course of treatment. In some embodiments, altering treatment comprises administering a different treatment and/or increasing or decreasing the dosage and/or frequency of administration of the therapeutic agent.

In some embodiments, the increase in likelihood of slowing, reversing, and/or halting the progression of PD in the subject is based on the relative amount by which the expression level of NF-kB and/or calcineurin decreased compared to the baseline expression level. For instance, in some embodiments, a greater relative decrease in expression level of NF-kB and/or calcineurin results in a higher likelihood of slowing, reversing, and/or halting the progression of PD in the subject, as compared to a lesser relative decrease in expression level of NF-kB and/or calcineurin.

In some embodiments, the increase in likelihood of slowing, reversing, and/or halting the progression of PD in the subject is based on the relative amount by which the activation level of the one or more canonical pathways increased compared to the baseline expression level. For instance, in some embodiments, a greater relative increase in activation level of the one or more canonical pathways results in a higher likelihood of slowing, reversing, and/or halting the progression of PD in the subject, as compared to a lesser relative increase in activation level of the one or more canonical pathways.

### 3. Methods for Treating Parkinson's Disease

Also provided herein are methods for treating Parkinson's Disease (PD) in a subject, the method comprising: a) determining a baseline expression level of NF-kB and/or calcineurin in a biological sample from a subject having PD; b) administering a therapeutic agent for the treatment of PD to the subject; c) determining an expression level of NF-kB and/or calcineurin in a biological sample from the subject at one or more time points following initiation of the treatment; and d) determining if the expression level of NF-kB and/or calcineurin at the one or more time points following initiation of the treatment decreased compared to the baseline expression level, wherein a decrease in the expression level of NF-kB and/or calcineurin at the one or more time points following initiation of the treatment decreased compared to the baseline expression level indicates an increased likelihood of therapeutic efficacy in treating PD in the subject.

Also provided herein are methods for treating Parkinson's Disease (PD) in a subject, the method comprising: a) determining a baseline expression level of NF-kB and/or calcineurin in a biological sample from a subject having PD; b) administering a therapeutic agent for the treatment of PD to the subject; c) determining an expression level of NF-kB and/or calcineurin in a biological sample from the subject at one or more time points following initiation of the treatment; and d) comparing the expression level of NF-kB and/or calcineurin at the one or more time points following initiation of the treatment to the baseline expression level, wherein the comparison indicates a likelihood of therapeutic efficacy in treating PD in the subject.

Also provided herein are therapeutic agents for use in the treatment of Parkinson's Disease (PD) in a subject, wherein the therapeutic agent is for use in a method comprising: a) determining a baseline expression level of NF-kB and/or calcineurin in a biological sample from a subject having PD; b) determining an expression level of NF-kB and/or calcineurin in a biological sample from the subject at one or more time points following administration of the treatment; and c) comparing the expression level of NF-kB and/or calcineurin at the one or more time points following initiation of the treatment to the baseline expression level, wherein the comparison indicates a likelihood of therapeutic efficacy in treating PD in the subject.

Also provided herein are uses of a therapeutic agent in the treatment of Parkinson's Disease (PD) in a subject, wherein the use comprises: a) determining a baseline expression level of NF-kB and/or calcineurin in a biological sample from a subject having PD; b) determining an expression level of NF-kB and/or calcineurin in a biological sample from the subject at one or more time points following administration of the therapeutic agent; and c) comparing the expression level of NF-kB and/or calcineurin at the one or more time points following initiation of the treatment to the baseline expression level, wherein the comparison indicates a likelihood of therapeutic efficacy in treating PD in the subject.

In some embodiments, administering a therapy comprising a therapeutic agent for the treatment of PD to the subject comprises administering the therapeutic agent at a therapeutically effective amount.

In some embodiments, the comparison indicates that the expression level of NF-kB and/or calcineurin at the one or more time points following initiation of treatment decreased compared to the baseline expression level.

In some embodiments, if the expression level of NF-kB and/or calcineurin at the one or more time points following initiation of treatment decreased compared to the baseline expression level, the comparison indicates an increased likelihood of therapeutic efficacy for the treatment of PD in the subject and/or indicates an increased degree of effectiveness at treating PD in the subject. In some embodiments, if the expression level of NF-kB and/or calcineurin at the one or more time points following initiation of treatment did not decrease compared to the baseline expression level, the comparison indicates a neutral or decreased likelihood of therapeutic efficacy for the treatment of PD in the subject and/or indicates a neutral or decreased degree of effectiveness at treating PD in the subject.

In some embodiments, a decreased expression level of NF-kB and/or calcineurin at the one or more time points following initiation of the treatment compared to the baseline expression level indicates an increase in likelihood of therapeutic efficacy in treating PD in the subject.

In some embodiments, the method further comprises continuing treatment if the comparison indicates an increased likelihood of therapeutic efficacy. In some embodiments, te method further comprises continuing treatment if the expression level of NF-kB and/or calcineurin at the one or more time points following initiation of the treatment decreased compared to the baseline expression level. In some embodiments, the method further comprises discontinuing treatment if the expression level of NF-kB and/or calcineurin at the one or more time points following initiation of the treatment did not decrease compared to the baseline expression level.

In some embodiments, treatment is continued if the expression level of NF-kB and/or calcineurin have decreased in the biological sample at the one or more time points following initiation of the therapy compared to the baseline expression level; or treatment is discontinued or altered if the expression level of NF-kB and/or calcineurin have not decreased in the biological sample at the one or more time points following initiation of the therapy compared to the baseline expression level. In some embodiments, altering the treatment comprises administering a different treatment, e.g., a different therapeutic agent, and/or comprises increasing or decreasing the dosing and/or frequency of the therapeutic agent.

Also provided herein are methods for treating Parkinson's Disease (PD) in a subject, the method comprising: a) determining a baseline expression level of NF-kB and/or calcineurin in a biological sample from a subject having PD; b) administering a therapeutic agent for the treatment of PD to the subject; c) determining an expression level of NF-kB and/or calcineurin in a biological sample from the subject at one or more time points following initiation of the treatment; and d) determining if the expression level of NF-kB and/or calcineurin at the one or more time points following initiation of the treatment decreased compared to the baseline expression level, wherein a decrease in the expression level of NF-kB and/or calcineurin at the one or more time points following initiation of the treatment decreased compared to the baseline expression level indicates an increased likelihood of therapeutic efficacy in treating PD in the subject; wherein: (i) if the expression level of NF-kB and/or calcineurin at the one or more time points following initiation of treatment decreased compared to the baseline expression level, the comparison indicates an increased likelihood of therapeutic efficacy for the treatment of PD in the subject and/or indicates an increased degree of effectiveness at treating PD in the subject; and/or (ii) if the expression level of NF-kB and/or calcineurin at the one or more time points following initiation of treatment did not decrease compared to the baseline expression level, the comparison indicates a neutral or decreased likelihood of therapeutic efficacy for the treatment of PD in the subject and/or indicates a neutral or decreased degree of effectiveness at treating PD in the subject.

Also provided herein are methods for treating Parkinson's Disease (PD) in a subject, the method comprising: a) determining a baseline activation level of one or more canonical pathways in a biological sample from a subject having PD, wherein the one or more canonical pathways are associated with cellular immune response signaling, neuroinflammation signaling, or PD signaling pathways; b) administering a therapy comprising a therapeutic agent for the treatment of PD to the subject; c) determining an activation level of one or more canonical pathways in a biological sample from the subject at one or more time points following initiation of the treatment; and d) comparing the activation level of the one or more canonical pathways at the one or more time points following initiation of treatment to the baseline expression level, wherein an increase in the activation level of the one or more canonical pathways indicates an increased likelihood of therapeutic efficacy.

Also provided herein are therapeutic agents for use in the treatment of Parkinson's Disease (PD) in a subject, wherein the therapeutic agent is for use in a method comprising: a) determining a baseline activation level of one or more canonical pathways in a biological sample from a subject having PD, wherein the one or more canonical pathways are associated with cellular immune response signaling, neuroinflammation signaling, or PD signaling pathways; b) determining an activation level of one or more canonical pathways in a biological sample from the subject at one or more time points following administration of the treatment; and c) comparing the activation level of one or more canonical pathways in a biological sample from the subject at the one or more time points following initiation of the treatment to the baseline expression level, wherein the comparison indicates a likelihood of therapeutic efficacy in treating PD in the subject.

Also provided herein are use of a therapeutic agent in the treatment of Parkinson's Disease (PD) in a subject, wherein the use comprises: a) determining a baseline activation level of one or more canonical pathways in a biological sample from a subject having PD, wherein the one or more canonical pathways are associated with cellular immune response signaling, neuroinflammation signaling, or PD signaling pathways; b) determining an activation level of one or more canonical pathways in a biological sample from the subject at one or more time points following administration of the treatment; and c) comparing the activation level of one or more canonical pathways in the biological sample from the subject at the one or more time points following initiation of the treatment to the baseline expression level, wherein the comparison indicates a likelihood of therapeutic efficacy in treating PD in the subject.

In some embodiments, the method further comprises continuing treatment if the activation level of the one or more canonical pathways indicates an increased likelihood of therapeutic efficacy. In some embodiments, treatment is continued if the activation level of the one or more canonical pathways have increased in the biological sample at the one or more time points following initiation of the therapy compared to the baseline activation level; or treatment is discontinued or altered if the activation level of the one or more canonical pathways have not increased in the biological sample at the one or more time points following initiation of the therapy compared to the baseline activation level. In some embodiments, altering the treatment comprises administering a different treatment, e.g., a different therapeutic agent, and/or comprises increasing or decreasing the dosing and/or frequency of the therapeutic agent.

In some embodiments, treatment is continued if the activation level of at least a threshold percentage of the one or more canonical pathways have increased in the biological sample at the one or more time points following initiation of the therapy compared to the baseline activation level. In some embodiments, the activation level of at least a threshold percentage of the one or more canonical pathways have increased in the biological sample at the one or more time points following initiation of the therapy compared to the baseline activation level. In some embodiments, the threshold percentage is at least 50%, at least 55%, at least 60%, at least 65%, at least 70%, at least 75%, at least 80%, at least 85%, at least 90%, or at least 95%.

In some embodiments, treatment is continued if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level. In some embodiments, the activation level of the one or more canonical pathways indicates an increased likelihood of therapeutic efficacy if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.

In some embodiments, an increase in likelihood of therapeutic efficacy in treating PD in the subject, is used as a measure for deciding whether to continue, discontinue, or alter the course of treatment.

In some embodiments, the increase in likelihood of therapeutic efficacy in treating PD in the subject is based on the relative amount by which the expression level of NF-kB and/or calcineurin decreased compared to the baseline expression level. For instance, in some embodiments, a greater relative decrease in expression level of NF-kB and/or calcineurin results in a higher likelihood of therapeutic efficacy in treating PD in the subject, as compared to a lesser relative decrease in expression level of NF-kB and/or calcineurin.

In some embodiments, the increase in likelihood of therapeutic efficacy in treating PD in the subject is based on the relative amount by which the activation level of the one or more canonical pathways increased compared to the baseline expression level. For instance, in some embodiments, a greater relative increase in activation level of the one or more canonical pathways results in a higher likelihood of therapeutic efficacy in treating PD in the subject, as compared to a lesser relative increase in activation level of the one or more canonical pathways.

### 4. Therapeutic Agents

Provided herein are therapeutic agents for use in the methods, uses, compositions, and kits described herein, including any of the methods described in Section I.A.1-3 and any of the compositions or kits described in Section I.B.

The methods provided herein involve treatment with a therapeutic agent for treating PD. The therapeutic agent can, in some embodiments, be any therapeutic agent used for the treatment of PD, such as any therapeutic agent known or expected to be effective in the treatment of PD.

In some embodiments, the therapeutic agent is selected from the group consisting of granulocyte macrophage colony stimulating factor (GM-CSF) or an analog thereof, GM-CSF mRNA, vasoactive intestinal peptide (VIP) or an analog thereof, and VIP mRNA.

In some embodiments, the GM-CSF or analog thereof is administered at or about 1 μg/kg/day, at or about 2 μg/kg/day, at or about 3 μg/kg/day, at or about 4 μg/kg/day, at or about 5 μg/kg/day, at or about 6 μg/kg/day, at or about 7 μg/kg/day, at or about 8 μg/kg/day, at or about 9 μg/kg/day, or at or about 10 μg/kg/day. In some embodiments, the GM-CSF or analog thereof is administered at a dose of between 0.1-10 μg/kg/day, or between 1-10 μg/kg/day, or between 2-8 μg/kg/day, or between 3-6 μg/kg/day. In some embodiments, the GM-CSF or analog thereof is administered at 3 μg/kg/day or 6 μg/kg/day.

In some embodiments, the GM-CSF or analog thereof is sargramostim. Sargramostim is a recombinant human GM-CSF that functions as an immunostimulator. In some embodiments, the sargramostim is administered subcutaneously five times per week, such as five days of administration in a row followed by two days without administration. In some embodiments, the sargramostim is administered at or about 1 μg/kg/day, at or about 2 μg/kg/day, at or about 3 μg/kg/day, at or about 4 μg/kg/day, at or about 5 μg/kg/day, at or about 6 μg/kg/day, at or about 7 μg/kg/day, at or about 8 μg/kg/day, at or about 9 μg/kg/day, or at or about 10 μg/kg/day. In some embodiments, the sargramostim is administered at a dose of between 0.1-10 μg/kg/day, or between 1-10 μg/kg/day, or between 2-8 μg/kg/day, or between 3-6 μg/kg/day. In some embodiments, the sargramostim is administered at 3 μg/kg/day or 6 μg/kg/day.

In some embodiments, the GM-CSF or analog thereof is molgramostim. Molgramostim is a recombinant human GM-CSF that functions as an immunostimulator. In some embodiments, the molgramostim is administered subcutaneously five times per week, such as five days of administration in a row followed by two days without administration. In some embodiments, the molgramostim is administered at or about 1 μg/kg/day, at or about 2 μg/kg/day, at or about 3 μg/kg/day, at or about 4 μg/kg/day, at or about 5 μg/kg/day, at or about 6 μg/kg/day, at or about 7 μg/kg/day, at or about 8 μg/kg/day, at or about 9 μg/kg/day, or at or about 10 μg/kg/day. In some embodiments, the molgramostim is administered at a dose of between 0.1-10 μg/kg/day, or between 1-10 μg/kg/day, or between 2-8 μg/kg/day, or between 3-6 μg/kg/day. In some embodiments, the molgramostim is administered at 3 μg/kg/day or 6 μg/kg/day.

In some embodiments, the VIP or analog thereof is aviptadil. Aviptadil is a synthetic form of human VIP.

In some embodiments, the GM-CSF mRNA encodes sargramostim or molgramostim.

In some embodiments, the VIP mRNA encodes aviptadil.

In some embodiments, the therapeutic agent is a vaccine, such as a vaccine for protection against the development and/or progression of PD.

In some embodiments, the therapeutic agent is an immunogen that induces a humoral immune response against at least one abnormal protein of PD. Exemplary immunogens that induces a humoral immune response against at least one abnormal protein of PD include those as described in U.S. Pat. No. 8,491,890 B2, the contents of which are hereby incorporated by reference in their entirety. In some embodiments, the immunogen is nitrated alpha synuclein or a fragment thereof. In some embodiments, the immunogen is a nitrated alpha synuclein fragment, and wherein the nitrated alpha synuclein fragment is a carboxy terminal fragment consisting of the carboxy terminal 20 amino acids of alpha synuclein up to the carboxy terminal 70 amino acids of alpha synuclein. In some embodiments, the immunogen is human nitrated alpha synuclein or a fragment thereof. In some embodiments, the nitrated alpha synuclein comprises the amino acid sequence of SEQ ID NO: 4, or comprises an amino acid sequence having at least 90%, 91%, 92%, 93%, 94%, 95%, 96%, 97%, 98%, or 99% sequence identity to the amino acid sequence of SEQ ID NO: 4. In some embodiments, the nitrated alpha synuclein fragment comprises amino acid residues 101-140 of SEQ ID NO: 4, or comprises an amino acid sequence having at least 90%, 91%, 92%, 93%, 94%, 95%, 96%, 97%, 98%, or 99% sequence identity to amino acid residues 101-140 of the amino acid sequence of SEQ ID NO: 4. In some embodiments, the nitrated alpha synuclein comprises the amino acid sequence of SEQ ID NO: 4.

In some embodiments, the immunogen is comprised in a composition that further comprises at least one adjuvant that stimulates regulatory T cells. In some embodiments, the adjuvant is selected from the group consisting of VIP, vitamin D, GM-CSF, and transforming growth factor beta (TGFβ). In some embodiments, the composition further comprises a pharmaceutically acceptable carrier.

In some embodiments, the therapeutic agent is a composition comprising an immunogen that induces a humoral immune response against at least one abnormal protein of PD; at least one adjuvant that stimulates regulatory T cells; and, optionally, a pharmaceutically acceptable carrier. In some embodiments, the therapeutic agent is a composition comprising an immunogen that induces a humoral immune response against at least one abnormal protein of PD; at least one adjuvant that stimulates regulatory T cells; and a pharmaceutically acceptable carrier. In some embodiments, the therapeutic agent is a composition comprising an immunogen that induces a humoral immune response against at least one abnormal protein of PD; and a pharmaceutically acceptable carrier.

In some embodiments, the therapeutic agent is an agent that reacts with and/or induces molecules that react with a GM-CSF receptor or a VIP receptor. In some embodiments, the GM-CSF receptor is CSF2R and/or the VIP receptor is VIP2R.

In some embodiments, the therapeutic agent is an agonist of granulocyte-macrophage colony stimulating factor 2 receptor (CSF2R) or vasoactive intestinal peptide receptor 2 (VIP2R). In some embodiments, the agonist is a peptide or peptide-like agonist of CSF2R or VIP2R. In some embodiments, the agonist is an antibody or a fragment thereof that binds to CSF2R or VIP2R.

In some embodiments, the therapeutic agent is a mimetic of GM-CSF or is a mimetic of VIP

In some embodiments, the therapeutic agent is levodopa. Levodopa is a precursor to dopamine. Levodopa is commonly used as a dopamine replacement agent, such as for the treatment of PD.

In some embodiments, the therapeutic agent is a dopamine receptor agonist. In some embodiments, the dopamine receptor agonist is selected from the group consisting of apomorphine, pramipexole, ropinirole, bromocriptine, cabergoline, pergolide, rotigotine, ciladopa, dihydrostine, dihydroergocryptine, dinapsoline, polyxanthine (doxanthrine), epicriptine, lisuride, pergolide, piribedil, pramipexole, propylnorapomorphine (propylnorapomorphine), quinagolide, roxburgine, ropinirole, rotigotine, rocindoline, sumanirole, and pharmaceutically acceptable salts, solvates and prodrugs thereof. In some embodiments, the dopamine receptor agonist is apomorphine. In some embodiments, the dopamine receptor agonist is pramipexole. In some embodiments, the dopamine receptor agonist is ropinirole. In some embodiments, the dopamine receptor agonist is bromocriptine. In some embodiments, the dopamine receptor agonist is cabergoline. In some embodiments, the dopamine receptor agonist is pergolide. In some embodiments, the dopamine receptor agonist is rotigotine. In some embodiments, the dopamine receptor agonist is ciladopa. In some embodiments, the dopamine receptor agonist is dihydrostine. In some embodiments, the dopamine receptor agonist is dihydroergocryptine. In some embodiments, the dopamine receptor agonist is dinapsoline. In some embodiments, the dopamine receptor agonist is polyxanthine (doxanthrine). In some embodiments, the dopamine receptor agonist is epicriptine. In some embodiments, the dopamine receptor agonist is lisuride. In some embodiments, the dopamine receptor agonist is pergolide. In some embodiments, the dopamine receptor agonist is piribedil. In some embodiments, the dopamine receptor agonist is pramipexole. In some embodiments, the dopamine receptor agonist is propylnorapomorphine (propylnorapomorphine). In some embodiments, the dopamine receptor agonist is quinagolide. In some embodiments, the dopamine receptor agonist is roxburgine. In some embodiments, the dopamine receptor agonist is ropinirole. In some embodiments, the dopamine receptor agonist is rotigotine. In some embodiments, the dopamine receptor agonist is rocindoline. In some embodiments, the dopamine receptor agonist is sumanirole.

In some embodiments, the administration of a therapeutic agent comprises administering the therapeutic agent at a therapeutically effective amount.

### 5. Biomarkers

Provided herein are biomarkers for use in any of the methods, uses, compositions, and kits described herein, including any of the methods described in Section I.A.1-3 and any of the compositions or kits described in Section I.B.

In some embodiments, the biomarkers comprise nuclear factor kappa B (NF-κB) and/or calcineurin. In some embodiments, the biomarker is NF-κB. In some embodiments, the biomarker is calcineurin. In some embodiments, the biomarkers are NF-κB and calcineurin. In some embodiments, the reduced expression of biomarkers, e.g., NF-κB and/or calcineurin, is indicative of the therapeutic efficacy of the therapeutic agent and/or of the progression of PD during the treatment.

High calcineurin activity is found to drive a toxic response in the presence of high α-synuclein levels in PD (Caraveo G. et al., Proc Natl Acad Sci USA 2014; 111 (34): E3544-52). Additionally, activation of the nuclear factor of activated T cells (NFAT) pathway which plays important roles in T cell activation and modulation of immune responses is altered (Hermann-Kleiter N. et al., Blood 2010; 115 (15): 2989-97). Most NFAT proteins are known to be regulated by calcineurin, and altered calcineurin/NFAT activation has been linked to the pathology of several neurodegenerative diseases including PD (Hogan P G. et al., Genes Dev 2003; 17 (18): 2205-32, Kipanyula M J. et al., J Aging Res 2016; 2016: 5081021). Thus, reducing calcineurin activity during treatment suggests a mechanism by which a therapeutic agent, such as sargramostim, can provide a therapeutic benefit to PD subjects. As such, identifying the subjects in which the therapeutic agent has reduced calcineurin is advantageous, such as for determining the therapeutic efficacy of a therapeutic treatment and/or monitoring the progression of PD during therapeutic treatment and/or informing treatment decisions, e.g., whether to continue or discontinue or alter treatment.

In both microglia and astroglia, activation of NF-kB, along with other proinflammatory transcription factors, leads to the transcription of several proinflammatory molecules (Liu X. et al., J Biol Chem 2002; 277 (42): 39312-9, Dasgupta S. et al., J Biol Chem 2003; 278 (25): 22424-31) that contribute or are causal to the loss of dopaminergic neurons in MPTP-intoxicated mice and PD patients (Nagatsu T. et al., J Neural Transm Suppl 2000; (60): 277-90, Mogi M. et al., Neurosci Lett 1994; 180 (2): 147-50). Additional studies have shown that inhibition of NF-κB activation reduces the induction of proinflammatory molecules and significantly protects nigrostriatal neurons against MPTP-induced neurodegeneration (Ghosh A. et al., Proc Natl Acad Sci USA 2007; 104 (47): 18754-9). Therefore, reduction of NF-κB activity by a therapeutic agent, such as sargramostim, may reduce the inflammation-mediated neurodegeneration and provide a consequent therapeutic benefit in PD subjects. As such, identifying the subjects in which the therapeutic agent has reduced NF-κB expression is advantageous, such as for determining the therapeutic efficacy of a therapeutic treatment and/or monitoring the progression of PD during therapeutic treatment and/or informing treatment decisions, e.g., whether to continue or discontinue or alter treatment.

For at least these collective reasons, identifying the subjects in which the therapeutic agent has reduced the expression level of NF-κB and/or calcineurin is advantageous, such as for determining the therapeutic efficacy of a therapeutic treatment and/or monitoring the progression of PD during therapeutic treatment and/or informing treatment decisions, e.g., whether to continue or discontinue or alter treatment.

Accordingly, in some embodiments, the biomarker is reduced expression of NF-κB and/or calcineurin. In some embodiments, the biomarker is reduced expression of NF-κB. In some embodiments, the biomarker is reduced expression of calcineurin. In some embodiments, the biomarker is reduced expression of NF-κB and calcineurin. In some embodiments, the expression level of NF-κB and/or calcineurin is determined prior to initiation of a treatment comprising a therapeutic agent, e.g., the baseline expression level of NF-κB and/or calcineurin is determined.

Accordingly, in some embodiments, the method comprises determining a baseline expression level of NF-kB and/or calcineurin in a biological sample from a subject having PD. In some embodiments, the method comprises determining an expression level of NF-kB and/or calcineurin in a biological sample from the subject at one or more time points following initiation of treatment with a therapeutic agent. In some embodiments, the method comprises determining a baseline expression level of NF-kB and/or calcineurin in a biological sample from a subject having PD; and determining an expression level of NF-kB and/or calcineurin in a biological sample from the subject at one or more time points following initiation of treatment with a therapeutic agent.

In some embodiments, the baseline expression level of NF-kB and/or calcineurin in a biological sample from a subject having PD is determined prior to initiation of a treatment comprising a therapeutic agent. In some embodiments, the baseline expression level is determined prior to initiation of the treatment. In some embodiments, the baseline expression level is determined at any time point prior to initiation of the treatment.

In some embodiments comprising administration of a therapy, determining the baseline expression level of NF-kB and/or calcineurin occurs prior to administering the therapy, which is also referred to as initiating the treatment.

In some embodiments, the baseline expression level of NF-kB and/or calcineurin is determined at or at least 1 day, at or at least 2 days, at or at least 3 days, at or at least 4 days, at or at least 5 days, at or at least 6 days, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment. In some embodiments, the baseline expression level of NF-kB and/or calcineurin is determined at or about 2 months prior to initiation of treatment. In some embodiments, the baseline expression level of NF-kB and/or calcineurin is determined between about 1 month and about 3 months prior to initiation of treatment. In some embodiments, the baseline expression level is determined on the same day that treatment is initiated.

In some embodiments, the determining an expression level of NF-kB and/or calcineurin in a biological sample from the subject at one or more time points following initiation of treatment with a therapeutic agent can be done at any one or more time points following initiation of treatment. In some embodiments, the one or more time points following initiation of treatment with the therapeutic agent can be any time point following initiation of the treatment.

In some embodiments, the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment. In some embodiments, the one or more time points following initiation of treatment with the therapeutic agent comprises a time point that is at or about 1 day, at or about 2 days, at or about 3 days, at or about 4 days, at or about 5 days, at or about 6 days, or at or about or 7 days or more following initiation of treatment. In some embodiments, the one or more time points following initiation of treatment with the therapeutic agent comprises one or more time points selected from the group consisting of a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment.

In some embodiments, the one or more time points following initiation of treatment with the therapeutic agent comprises a time point at or about 2 months following initiation of treatment. In some embodiments, the one or more time points following initiation of treatment with the therapeutic agent comprises a time point at or about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 days following initiation of treatment.

In some embodiments, the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment. In some embodiments, the one or more time points following initiation of treatment with the therapeutic agent comprises one or more time points selected from the group consisting of a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.

In some embodiments, the one or more time points following initiation of treatment with the therapeutic agent comprises at least 1, at least 2, at least 3, at least 4, at least 5, at least 6, at least 7, at least 8, at least 9, at least 10, at least 11, or at least 12 different time points following initiation of treatment with the therapeutic agent. In some embodiments, each of the different time points is separated by an interval of time. In some embodiments, the internal of time is or is about 1 week, is or is about 2 weeks, is or is about 3 weeks, is or is about 4 weeks, is or is about 1 month, is or is about 5 weeks, is or is about 6 weeks, is or is about 7 weeks, is or is about 8 weeks, or is or is about 2 months.

In some embodiments, the expression level of the biomarker, e.g., NF-kB and/or calcineurin, can be determined using any appropriate assay or method available in the art. In some embodiments, the expression level of NF-kB and/or calcineurin is determined by an assay. In some embodiments, the assay is selected from the group consisting of mass spectrometry, enzyme-linked immunosorbent assay (ELISA), western blotting, or polymerase chain reaction (PCR). In some embodiments, the expression level of NF-kB and/or calcineurin is determined by an ELISA assay. In some embodiments, the assay is an immunoassay or an mRNA expression assay. In some embodiments, the expression level of NF-kB and/or calcineurin is determined by PCR. In some embodiments, the expression level of NF-kB and/or calcineurin is determined by real-time PCR. In some embodiments, the assay is a gene expression assay, e.g., an mRNA expression assay. In some embodiments, the assay is a gene expression assay, such as a microarray.

In some embodiments, the biomarkers comprise one or more pathways, such as one or more canonical pathways.

Accordingly, in some embodiments, the method comprises determining a baseline activation level of one or more canonical pathways in a biological sample from a subject having PD.

In some embodiments, the method comprises determining a baseline activation level of one or more canonical pathways in a biological sample from a subject having PD. In some embodiments, the method comprises determining an activation level of the one or more canonical pathways in a biological sample from the subject at one or more time points following initiation of treatment with a therapeutic agent. In some embodiments, the method comprises determining a baseline activation level of one or more canonical pathways in a biological sample from a subject having PD; and determining an activation level of the one or more canonical pathways in a biological sample from the subject at one or more time points following initiation of treatment with a therapeutic agent.

In some embodiments, the baseline activation level is determined at or at least 1 day, at or at least 2 days, at or at least 3 days, at or at least 4 days, at or at least 5 days, at or at least 6 days, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment. In some embodiments, the baseline activation level is determined on the same day that treatment is initiated.

In some embodiments, the one or more time points following initiation of treatment with the therapeutic agent can be any time point following initiation of the treatment.

In some embodiments, the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment. In some embodiments, the one or more time points following initiation of treatment with the therapeutic agent comprises a time point that is at or about 1 day, at or about 2 days, at or about 3 days, at or about 4 days, at or about 5 days, at or about 6 days, or at or about or 7 days or more following initiation of treatment. In some embodiments, the one or more time points following initiation of treatment with the therapeutic agent comprises one or more time points selected from the group consisting of a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment.

In some embodiments, the one or more time points following initiation of treatment with the therapeutic agent comprises a time point at or about 2 months following initiation of treatment.

In some embodiments, the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment. In some embodiments, the one or more time points following initiation of treatment with the therapeutic agent comprises one or more time points selected from the group consisting of a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.

In some embodiments, the one or more time points following initiation of treatment with the therapeutic agent comprises at least 1, at least 2, at least 3, at least 4, at least 5, at least 6, at least 7, at least 8, at least 9, at least 10, at least 11, or at least 12 different time points following initiation of treatment with the therapeutic agent. In some embodiments, each of the different time points is separated by an interval of time. In some embodiments, the internal of time is or is about 1 week, is or is about 2 weeks, is or is about 3 weeks, is or is about 4 weeks, is or is about 1 month, is or is about 5 weeks, is or is about 6 weeks, is or is about 7 weeks, is or is about 8 weeks, or is or is about 2 months.

In some embodiments, the activation level of the one or more canonical pathways can be determined using any appropriate assay or method available in the art for determining activation level of a pathway or a component thereof. In some embodiment, the activation level of the one or more canonical pathways is determined using gene expression data, such as from a microarray or next generation sequencing methods. In some embodiment, the activation level of the one or more canonical pathways is determined using ELISA and/or western blotting methods. In some embodiments, the activation level of the one or more canonical pathways is determined using bioinformatics analysis. The bioinformatics analysis can be any suitable bioinformatics analysis, e.g., using gene expression data, such as from a microarray. In some embodiments, the activation level of the one or more canonical pathways is determined using the ingenuity pathway analysis (IPA) method. IPA is a web-based bioinformatics method that uses data, such as gene expression data from microarray or next-generation sequencing methods, to analyze the data and identify networks and signaling pathways relevant to the data. For instance, IPA can be used to identify pathways that have an activation level that is increased in one sample compared to another sample, such as from a baseline sample taken from a subject prior to treatment compared to a sample taken from the same subject at a time point following the initiation of treatment.

In some embodiments, the comparing the activation level of the one or more canonical pathways at the one or more time points following initiation of treatment to the baseline activation level comprises determining whether the activation level of the one or more canonical pathways at the one or more time points following initiation of treatment increased compared to the baseline activation level.

In some embodiments, the activation level of at least a threshold percentage of the one or more canonical pathways have increased in the biological sample at the one or more time points following initiation of the therapy compared to the baseline activation level. In some embodiments comprising administration of a treatment, treatment is continued if the activation level of at least a threshold percentage of the one or more canonical pathways have increased in the biological sample at the one or more time points following initiation of the therapy compared to the baseline activation level. In some embodiments comprising administration of a treatment, the activation level of at least a threshold percentage of the one or more canonical pathways have increased in the biological sample at the one or more time points following initiation of the therapy compared to the baseline activation level.

In some embodiments, the threshold percentage is at least 50%, at least 55%, at least 60%, at least 65%, at least 70%, at least 75%, at least 80%, at least 85%, at least 90%, or at least 95%. In some embodiments, the comparison indicates an increase in likelihood of therapeutic efficacy for the treatment of PD in the subject and/or indicates an increase in degree of effectiveness at treating PD in the subject if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.

In some embodiments, the one or more canonical pathways comprises a pathway associated with cellular immune response signaling, neuroinflammation signaling, and/or PD signaling, or any combination thereof.

In some embodiments, the one or more canonical pathways are selected from the group consisting of IL-8 Signaling, fMLP Signaling in Neutrophils, Role of NFAT in Regulation of the Immune Response, Fcγ Receptor-mediated Phagocytosis in Macrophages and Monocytes, CXCR4 Signaling, PKCθ Signaling in T Lymphocytes, Leukocyte Extravasation Signaling, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, CCR3 Signaling in Eosinophils, IL-3 Signaling, GM-CSF Signaling, Macropinocytosis Signaling, IL-7 Signaling Pathway, Interferon Signaling, Nur77 Signaling in T Lymphocytes, IL-9 Signaling, Antiproliferative Role of TOB in T Cell Signaling, CCR5 Signaling in Macrophages, Role of PKR in Interferon Induction and Antiviral Response, Production of Nitric Oxide and Reactive Oxygen Species in Macrophages, and iNOS Signaling, or any combination thereof.

In some embodiments, the one or more canonical pathways comprise IL-8 Signaling, fMLP Signaling in Neutrophils, Role of NFAT in Regulation of the Immune Response, Fcγ Receptor-mediated Phagocytosis in Macrophages and Monocytes, CXCR4 Signaling, PKCθ Signaling in T Lymphocytes, Leukocyte Extravasation Signaling, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, CCR3 Signaling in Eosinophils, IL-3 Signaling, GM-CSF Signaling, Macropinocytosis Signaling, IL-7 Signaling Pathway, Interferon Signaling, Nur77 Signaling in T Lymphocytes, IL-9 Signaling, Antiproliferative Role of TOB in T Cell Signaling, CCR5 Signaling in Macrophages, Role of PKR in Interferon Induction and Antiviral Response, Production of Nitric Oxide and Reactive Oxygen Species in Macrophages, and iNOS Signaling, or any combination thereof.

In some embodiments, the one or more canonical pathways is or comprises IL-8 Signaling. In some embodiments, the one or more canonical pathways is or comprises fMLP Signaling in Neutrophils. In some embodiments, the one or more canonical pathways is or comprises Role of NFAT in Regulation of the Immune Response. In some embodiments, the one or more canonical pathways is or comprises Fcγ Receptor-mediated Phagocytosis in Macrophages and Monocytes. In some embodiments, the one or more canonical pathways is or comprises CXCR4 Signaling. In some embodiments, the one or more canonical pathways is or comprises PKCθ Signaling in T Lymphocytes. In some embodiments, the one or more canonical pathways is or comprises Leukocyte Extravasation Signaling. In some embodiments, the one or more canonical pathways is or comprises CD28 Signaling in T Helper Cells. In some embodiments, the one or more canonical pathways is or comprises iCOS-iCOSL Signaling in T Helper Cells. In some embodiments, the one or more canonical pathways is or comprises Calcium-induced T Lymphocyte Apoptosis. In some embodiments, the one or more canonical pathways is or comprises Natural Killer Cell Signaling. In some embodiments, the one or more canonical pathways is or comprises PI3K Signaling in B Lymphocytes. In some embodiments, the one or more canonical pathways is or comprises CCR3 Signaling in Eosinophils. In some embodiments, the one or more canonical pathways is or comprises IL-3 Signaling. In some embodiments, the one or more canonical pathways is or comprises GM-CSF Signaling. In some embodiments, the one or more canonical pathways is or comprises Macropinocytosis Signaling. In some embodiments, the one or more canonical pathways is or comprises IL-7 Signaling Pathway. In some embodiments, the one or more canonical pathways is or comprises Interferon Signaling. In some embodiments, the one or more canonical pathways is or comprises Nur77 Signaling in T Lymphocytes. In some embodiments, the one or more canonical pathways is or comprises IL-9 Signaling. In some embodiments, the one or more canonical pathways is or comprises Antiproliferative Role of TOB in T Cell Signaling. In some embodiments, the one or more canonical pathways is or comprises CCR5 Signaling in Macrophages. In some embodiments, the one or more canonical pathways is or comprises Role of PKR in Interferon Induction and Antiviral Response. In some embodiments, the one or more canonical pathways is or comprises Production of Nitric Oxide and Reactive Oxygen Species in Macrophages. In some embodiments, the one or more canonical pathways is or comprises iNOS Signaling.

In some embodiments, the one or more canonical pathways comprise fMLP Signaling in Neutrophils, Role of NFAT in Regulation of the Immune Response, PKCθ Signaling in T Lymphocytes, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, IL-3 Signaling, GM-CSF Signaling, Nur77 Signaling in T Lymphocytes, IL-9 Signaling, Role of PKR in Interferon Induction and Antiviral Response, Production of Nitric Oxide and Reactive Oxygen Species in Macrophages, and iNOS Signaling, or any combination thereof. In some embodiments, the one or more canonical pathways are selected from the group consisting of fMLP Signaling in Neutrophils, Role of NFAT in Regulation of the Immune Response, PKCθ Signaling in T Lymphocytes, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, IL-3 Signaling, GM-CSF Signaling, Nur77 Signaling in T Lymphocytes, IL-9 Signaling, Role of PKR in Interferon Induction and Antiviral Response, Production of Nitric Oxide and Reactive Oxygen Species in Macrophages, and iNOS Signaling, or any combination thereof. In some embodiments, the one or more canonical pathways associated with cellular immune response signaling, neuroinflammation signaling, and/or PD signaling, or any combination thereof, are selected from the group consisting of fMLP Signaling in Neutrophils, Role of NFAT in Regulation of the Immune Response, PKCθ Signaling in T Lymphocytes, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, IL-3 Signaling, GM-CSF Signaling, Nur77 Signaling in T Lymphocytes, IL-9 Signaling, Role of PKR in Interferon Induction and Antiviral Response, Production of Nitric Oxide and Reactive Oxygen Species in Macrophages, and iNOS Signaling, or any combination thereof.

### 6. Subjects and Biological Samples

Provided herein are methods, uses, compositions, and kits for use with subjects having Parkinson's Disease (PD), including any of the methods described in Section I.A.1-3 and any of the compositions or kits described in Section I.B.

In some embodiments, the subject has PD. In some embodiments, the subject has been diagnosed with PD. In some embodiments, the subject is human. In some embodiments, the subject was diagnosed with PD at least 1 year, at least 2 years, or at least 3 years or more prior to the initiation of treatment. In some embodiments, the subject was diagnosed with PD between or between about 3 years and 15 years prior to the initiation of treatment. In some embodiments, the subject was diagnosed with PD between or between about 1 year and 20 years, between or between about 1 month and 20 years, or between or between about 1 year and 15 years prior to the initiation of treatment.

In some embodiments, the subject has signs and symptoms associated with PD. In some embodiments, the signs and symptoms associated with PD include one or more of asymmetric bradykinesia, resting tremor, and/or muscle rigidity persisting for longer than three years with less than stage four on the Hoehn and Yahr disease scale. In some embodiments, the subject does not exhibit signs and symptoms associated with PD.

In some embodiments, the subject is assessed for motor function. Motor function can be assessed prior to, concurrently with, and/or at one or more time points following initiation of the treatment. In some embodiments, motor function is assessed by determining a Movement Disorder Society-Unified Parkinson's Disease Rating Scale (MDS-UPDRS) score. In some embodiments, the MDS-UPDRS score is an MDS-UPDRS Part III score.

In some embodiments, an MDS-UPDRS Part III score is determined prior to administration or initiation of a treatment comprising a therapeutic agent. In some embodiments, an MDS-UPDRS Part III score is determined concurrently with administration or initiation of a treatment comprising a therapeutic agent. In some embodiments, an MDS-UPDRS Part III score is determined at one or more time points following initiation of a treatment comprising a therapeutic agent. In some embodiments, an MDS-UPDRS Part III score is determined prior to administration or initiation of a treatment comprising a therapeutic agent, and at one or more time points following initiation of the treatment. In some embodiments, the one or more time points following initiation of the treatment can be at any one or more time points between at or about 1 week and at or about 2 years or more following initiation of the treatment, such as between at or about 1 month and at or about 18 months, or between at or about 1 month and at or about 12 months, or between at or about 2 months and at or about 12 months following initiation of the treatment, or at any time point in between.

In some embodiments, an MDS-UPDRS Part III score is determined at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, or at least or at least about 3 months or more prior to administration or initiation of a treatment comprising a therapeutic agent.

In some embodiments, an MDS-UPDRS Part III score is determined at or about 1 month, at or about 2 months, at or about 3 months, at or about 4 months, at or about 5 months, at or about 6 months, at or about 7 months, at or about 8 months, at or about 9 months, at or about 10 months, at or about 11 months, at or about 12 months, or at least or at least about 12 months or more following initiation of a treatment comprising a therapeutic agent.

In some embodiments, the MDS-UPDRS Part III score is decreased at one or more time points following initiation of the treatment compared to the MDS-UPDRS Part III score prior to administration or initiation of the treatment. In some embodiments, the MDS-UPDRS Part III score is decreased by at least 5%, at least 10%, at least 15%, at least 20%, at least 25%, at least 30%, at least 35%, at least 40%, at least 45%, or at least 50% or more at one or more time points following initiation of the treatment compared to the MDS-UPDRS Part III score prior to administration or initiation of the treatment.

In some embodiments, the MDS-UPDRS Part III score is decreased at one or more time points selected from among 1 month, 2 months, 3 months, 4 months, 5 months, 6 months, 7 months, 8 months, 9 months, 10 months, 11 months, and 12 months following initiation of the treatment compared to the MDS-UPDRS Part III score prior to administration or initiation of the treatment. In some embodiments, the MDS-UPDRS Part III score is decreased at or about 3 months following initiation of the treatment compared to the MDS-UPDRS Part III score prior to administration or initiation of the treatment. In some embodiments, the MDS-UPDRS Part III score is decreased at or about 6 months following initiation of the treatment compared to the MDS-UPDRS Part III score prior to administration or initiation of the treatment. In some embodiments, the MDS-UPDRS Part III score is decreased at or about 8 months following initiation of the treatment compared to the MDS-UPDRS Part III score prior to administration or initiation of the treatment. In some embodiments, the MDS-UPDRS Part III score is decreased at one or more time points between or between about 3 and 12 months, or between or between about 3 and 8 months following initiation of the treatment compared to the MDS-UPDRS Part III score prior to administration or initiation of the treatment.

The methods provided herein also involve determining the expression level of NF-kB and/or calcineurin, and/or the activation level of one or more canonical pathways, in a biological sample from a subject having PD, such as described in Section I.A.1-5.

In some embodiments, the biological sample comprises white blood cells. In some embodiments, the biological sample comprises peripheral blood lymphocytes (PBLs). In some embodiments, the biological sample comprises T cells.

In some embodiments, the biological sample is an isolated biological sample. In some embodiments, the biological sample is obtained from the subject by leukapheresis. Leukapheresis is a procedure in which white blood cells are isolated from a blood sample. Accordingly, in some embodiments, the biological sample is a leukapheresis sample. In some embodiments, the biological sample is a leukapheresis sample that is further processed and/or subjected to isolation of one or more cell types.

### B. Compositions, Kits, and Articles of Manufacture

Provided here are compositions, kits, and articles of manufacture, each for carrying out any of the methods and uses provided herein, including any of the methods as described in Section I.A.

Also provided herein are compositions, kits, and articles of manufacture, each comprising a detection reagent for determining the expression level of NF-kB and/or calcineurin. In some embodiments, the detection reagent is or comprises one or more reagents for use in carrying out an assay selected from the group consisting of mass spectrometry, enzyme-linked immunosorbent assay (ELISA), western blotting, and polymerase chain reaction (PCR). In some embodiments, the assay is a gene expression assay, e.g., an mRNA expression assay.

Also provided herein are compositions, kits, and articles of manufacture, each comprising a detection reagent for determining the activation level of one or more canonical pathways, such as any of the canonical pathways described herein.

Also provided herein are compositions for detecting NF-kB and/or calcineurin expression in a biological sample, wherein the composition comprises a kit for a mass spectrometry analysis, an ELISA assay, a western blotting assay, or a PCR reaction. In some embodiments, the kit comprises a reagent for an ELISA assay. In some embodiments, the kit for the ELISA assay comprises a multi-well sample plate that is coated with immobilized capture antibodies that bind to NF-kB and/or calcineurin; detection antibodies covalently linked to an enzyme wherein the detection antibodies also bind to NF-kB and/or calcineurin; a colored or fluorescent product that will be catalyzed by the enzyme attached to the detection antibody; and appropriate buffers.

Also provided herein is a kit, comprising reagents for detecting NF-kB and/or calcineurin expression in a biological sample, and, optionally, instructions for detecting NF-kB and/or calcineurin expression in the biological sample.

Also provided herein is a kit, comprising reagents for determining the activation level of one or more canonical pathways in a biological sample, and, optionally, instructions for detecting NF-kB and/or calcineurin expression in the biological sample.

In some embodiments, the kit, composition, or article of manufacture comprises reagents comprising components for performing an assay for determining the expression level of NF-kB and/or calcineurin expression in a biological sample, and/or for determining the activation level of one or more canonical pathways. In some embodiments, the assay is an immunoassay or an mRNA expression assay.

Also provided herein are biomarkers for monitoring the progression of Parkinson's Disease (PD) and/or the effectiveness of therapeutics for the treatment of PD, wherein the biomarkers are NF-κB and calcineurin; or are one or more canonical pathways associated with cellular immune response signaling, neuroinflammation signaling, or PD signaling pathways.

In some embodiments, the one or more canonical pathways are selected from the group consisting of IL-8 Signaling, fMLP Signaling in Neutrophils, Role of NFAT in Regulation of the Immune Response, Fcγ Receptor-mediated Phagocytosis in Macrophages and Monocytes, CXCR4 Signaling, PKCθ Signaling in T Lymphocytes, Leukocyte Extravasation Signaling, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, CCR3 Signaling in Eosinophils, IL-3 Signaling, GM-CSF Signaling, Macropinocytosis Signaling, IL-7 Signaling Pathway, Interferon Signaling, Nur77 Signaling in T Lymphocytes, IL-9 Signaling, Antiproliferative Role of TOB in T Cell Signaling, CCR5 Signaling in Macrophages, Role of PKR in Interferon Induction and Antiviral Response, Production of Nitric Oxide and Reactive Oxygen Species in Macrophages, and iNOS Signaling. In some embodiments, the one or more canonical pathways are selected from the group consisting of fMLP Signaling in Neutrophils, Role of NFAT in Regulation of the Immune Response, PKCθ Signaling in T Lymphocytes, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, IL-3 Signaling, GM-CSF Signaling, Nur77 Signaling in T Lymphocytes, IL-9 Signaling, Role of PKR in Interferon Induction and Antiviral Response, Production of Nitric Oxide and Reactive Oxygen Species in Macrophages, and iNOS Signaling.

### II. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art

Although items, elements, or components of the subject matter may be described or claimed in the singular, the plural is contemplated to be within the scope thereof unless limitation to the singular is explicitly stated. For example, use of “a” or “an” should be understood to include both singular and plural unless explicitly stated. The presence of broadening words and phrases, such as “one or more,” “at least,” “but not limited to,” or other like phrases in some instances, shall not be read to mean that the narrower case is intended or required in instances where such broadening phrases may be absent.

The term “treat” as used herein refers to any type of treatment that imparts a benefit to a subject, e.g., patient, afflicted with the disease, including improvement in the disease of the subject, e.g., in one or more symptoms, or in slowing, halting, and/or reversing the progression of the disease, e.g., Parkinson's Disease.

A “therapeutically effective amount” of a therapeutic agent, or a compound, or a composition, e.g., a pharmaceutical composition, refers to an amount effective to prevent, inhibit, treat, or lessen the symptoms of a particular disorder or disease. The treatment of Parkinson's Disease (PD) herein may refer to curing, relieving, and/or preventing PD, a symptom(s) of it, or the predisposition towards it, or may refer to slowing, halting, and/or reversing the progression of PD.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxiliary agent, filler, disintegrant, lubricating agent, binder, stabilizer, preservative or vehicle with which an active agent of the present disclosure is administered. Pharmaceutically acceptable carriers can be sterile liquids, such as water and oils, including those of petroleum, animal, vegetable, or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present disclosure can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N. Y., 1980; and Kibbe, et al., Eds., Handbook of Pharmaceutical Excipients (3rd Ed.), American Pharmaceutical Association, Washington, 1999.

As used herein, “regulatory T cells” or “Tregs” are CD4+CD25+ cells that exhibit immunoinhibitory properties.

An “immunogen” refers to a compound comprising a peptide, polypeptide or protein which is “immunogenic,” i.e., capable of eliciting, augmenting or boosting an immune response (e.g., cellular and/or humoral). The immunogen can be recombinantly produced. An immunogen comprises at least one antigenic determinant or epitope.

### III. Exemplary Embodiments

Among the provided embodiments are:
1. A method of determining the efficacy of a therapeutic agent for the treatment of Parkinson's Disease (PD), the method comprising: a) determining a baseline expression level of NF-kB and/or calcineurin in a biological sample from a subject having PD; b) determining an expression level of NF-kB and/or calcineurin in a biological sample from the subject at one or more time points following initiation of treatment with a therapeutic agent; and c) determining if the expression level of NF-kB and/or calcineurin at the one or more time points following initiation of treatment decreased compared to the baseline expression level.
2. A method of determining the efficacy of a therapeutic agent for the treatment of Parkinson's Disease (PD), the method comprising: a) determining a baseline expression level of NF-kB and/or calcineurin in a biological sample from a subject having PD; b) determining an expression level of NF-kB and/or calcineurin in a biological sample from a subject at one or more time points following initiation of treatment with a therapeutic agent; and c) comparing the expression level of NF-kB and/or calcineurin at the one or more time points following initiation of treatment to the baseline expression level, wherein the comparison indicates a likelihood of therapeutic efficacy for the treatment of PD in the subject and/or indicates a degree of effectiveness at treating PD in the subject.
3. The method of embodiment 2, wherein the comparison indicates that the expression level of NF-kB and/or calcineurin at the one or more time points following initiation of treatment decreased compared to the baseline expression level.
4. The method of embodiment 2, wherein if the expression level of NF-kB and/or calcineurin at the one or more time points following initiation of treatment decreased compared to the baseline expression level, the comparison indicates an increased likelihood of therapeutic efficacy for the treatment of PD in the subject and/or indicates an increased degree of effectiveness at treating PD in the subject.
5 The method of embodiment 2, wherein if the expression level of NF-kB and/or calcineurin at the one or more time points following initiation of treatment did not decrease compared to the baseline expression level, the comparison indicates a neutral or decreased likelihood of therapeutic efficacy for the treatment of PD in the subject and/or indicates a neutral or decreased degree of effectiveness at treating PD in the subject.
6. The method of any one of embodiments 1-4, wherein a decreased expression level of NF-kB and/or calcineurin at the one or more time points following initiation of treatment compared to the baseline expression level indicates an increase in likelihood of therapeutic efficacy for the treatment of PD in the subject and/or indicates an increase in degree of effectiveness at treating PD in the subject.
7. The method of any one of embodiments 1-6, wherein the baseline expression level is determined at or at least 1 day, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment.
8. The method of any one of embodiments 1-7, wherein the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment.
9 The method of any one of embodiments 1-8, wherein the one or more time points following initiation of treatment with the therapeutic agent comprises a time point at or about 2 months following initiation of treatment.
10. The method of any one of embodiments 1-9, wherein the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.
11. The method of any one of embodiments 1-10, wherein the expression level of NF-kB and/or calcineurin is determined by an assay.
12. The method of embodiment 11, wherein the assay is selected from the group consisting of mass spectrometry, enzyme-linked immunosorbent assay (ELISA), western blotting, or polymerase chain reaction (PCR).
13. The method of embodiment 12, wherein the PCR is real-time PCR.
14. The method of any one of embodiments 1-13, wherein the expression level of NF-kB and/or calcineurin is decreased at the one or more time points following initiation of treatment compared to the baseline expression level.
15. A method of determining the efficacy of a therapeutic agent for the treatment of Parkinson's Disease (PD), the method comprising: a) determining a baseline activation level of one or more canonical pathways in a biological sample from a subject having PD, wherein the one or more canonical pathways are associated with cellular immune response signaling, neuroinflammation signaling, or PD signaling pathways; b) determining an activation level of the one or more canonical pathways in a biological sample from the subject at one or more time points following initiation of treatment with a therapeutic agent; and c) comparing the activation level of the one or more canonical pathways at the one or more time points following initiation of treatment to the baseline expression level, wherein an increase in the activation of the one or more canonical pathways at the one or more time points following initiation of treatment to the baseline expression level indicates an increase in likelihood of therapeutic efficacy for the treatment of PD and/or indicates an increase in degree of effectiveness at treating PD in the subject.
16. The method of embodiment 15, wherein the comparing the activation level of the one or more canonical pathways at the one or more time points following initiation of treatment to the baseline activation level indicates a likelihood of therapeutic efficacy for the treatment of PD and/or indicates a degree of effectiveness at treating PD in the subject.
17. The method of embodiment 15 or embodiment 16, wherein if the activation level of the one or more canonical pathways at the one or more time points following initiation of treatment to the baseline activation level increased, the comparison indicates an increased likelihood of therapeutic efficacy for the treatment of PD in the subject and/or indicates an increased degree of effectiveness at treating PD in the subject.
18. The method of any one of embodiments 15-17, wherein an increased activation level of the one or more canonical pathways at the one or more time points following initiation of treatment as compared to the baseline activation level indicates an increase in likelihood of therapeutic efficacy for the treatment of PD in the subject and/or indicates an increase in degree of effectiveness at treating PD in the subject.
19. The method of any one of embodiments 15-17, wherein the activation level of the one or more canonical pathways at the one or more time points following initiation of treatment as compared to the baseline activation level increased.
20. The method of any one of embodiments 15-19, wherein the activation level of at least a threshold percentage of the one or more canonical pathways have increased in the biological sample at the one or more time points following initiation of the therapy compared to the baseline activation level.
21. The method of embodiment 20, wherein the threshold percentage is at least 50%, at least 55%, at least 60%, at least 65%, at least 70%, at least 75%, at least 80%, at least 85%, at least 90%, or at least 95%.
22. The method of any one of embodiments 15-21, wherein the comparison indicates an increase in likelihood of therapeutic efficacy for the treatment of PD in the subject and/or indicates an increase in degree of effectiveness at treating PD in the subject if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.
23. A method of monitoring the progression of Parkinson's Disease (PD) during treatment, the method comprising: a) determining a baseline expression level of NF-kB and/or calcineurin in a biological sample from a subject having PD prior to initiation of a treatment comprising a therapeutic agent; b) determining an expression level of NF-kB and/or calcineurin in a biological sample from a subject at one or more time points following initiation of the treatment; and c) determining if the expression level of NF-kB and/or calcineurin at the one or more time points following initiation of the treatment decreased compared to the baseline expression level.
24. A method of monitoring the progression of Parkinson's Disease (PD) during treatment, the method comprising: a) determining a baseline expression level of NF-kB and/or calcineurin in a biological sample from a subject having PD prior to initiation of a treatment comprising a therapeutic agent; b) determining an expression level of NF-kB and/or calcineurin in a biological sample from a subject at one or more time points following initiation of the treatment; and c) comparing the expression level of NF-kB and/or calcineurin at the one or more time points following initiation of the treatment compared to the baseline expression level, wherein the comparison indicates a likelihood of slowing, reversing, and/or halting the progression of PD in the subject.
25. The method of embodiment 24, wherein the comparison indicates that the expression level of NF-kB and/or calcineurin at the one or more time points following initiation of treatment decreased compared to the baseline expression level.
26. The method of embodiment 24, wherein if the expression level of NF-kB and/or calcineurin at the one or more time points following initiation of treatment decreased compared to the baseline expression level, the comparison indicates an increased likelihood of slowing, reversing, and/or halting the progression of PD in the subject.
27. The method of embodiment 24, wherein if the expression level of NF-kB and/or calcineurin at the one or more time points following initiation of treatment did not decrease compared to the baseline expression level, the comparison indicates a neutral or decreased likelihood of slowing, reversing, and/or halting the progression of PD in the subject.
28. The method of any one of embodiments 23-27, wherein a decreased expression level of NF-kB and/or calcineurin at the one or more time points following initiation of treatment compared to the baseline expression level indicates an increased likelihood of slowing, reversing, and/or halting the progression of PD in the subject.
29. The method of any one of embodiments 23-28, wherein if the expression level of NF-kB and/or calcineurin at the one or more time points following initiation of treatment decreased compared to the baseline expression level, the therapeutic agent is indicated as being effective at slowing, reversing, and/or halting the progression of PD in the subject.
30. The method of any one of embodiments 23-29, wherein a decreased expression level of NF-kB and/or calcineurin at the one or more time points following initiation of treatment compared to the baseline expression level indicates an increase in likelihood of slowing, reversing, and/or halting the progression of PD in the subject and/or indicates an increase in degree of effectiveness at slowing, reversing, and/or halting the progression of PD in the subject.
31. The method of any one of embodiments 23-30, wherein the baseline expression level is determined at or at least 1 day, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment.
32. The method of any one of embodiments 23-31, wherein the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment.
33. The method of any one of embodiments 23-32, wherein the one or more time points following initiation of treatment with the therapeutic agent comprises a time point at or about 2 months following initiation of treatment.
34. The method of any one of embodiments 23-33, wherein the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.
35. The method of any one of embodiments 23-34, wherein the expression level of NF-kB and/or calcineurin is determined by an assay.
36. The method of embodiment 35, wherein the assay is selected from the group consisting of mass spectrometry, enzyme-linked immunosorbent assay (ELISA), western blotting, or polymerase chain reaction (PCR).
37. The method of embodiment 36, wherein the PCR is real-time PCR.
38. The method of any one of embodiments 23-37, wherein the expression level of NF-kB and/or calcineurin is decreased at the one or more time points following initiation of treatment compared to the baseline expression level.
39. A method of monitoring the progression of Parkinson's Disease (PD) during treatment, the method comprising: a) determining a baseline activation level of one or more canonical pathways in a biological sample from a subject having PD prior to initiation of a treatment comprising a therapeutic agent, wherein the one or more canonical pathways are associated with cellular immune response signaling, neuroinflammation signaling, or PD signaling pathways; b) determining an activation level of the one or more canonical pathways in a biological sample from a subject at one or more time points following initiation of the treatment; and c) comparing the activation level of the one or more canonical pathways at the one or more time points following initiation of treatment to the baseline expression level, wherein an increase in the activation of the one or more canonical pathways at the one or more time points following initiation of treatment compared to the baseline expression level indicates an increase in likelihood of slowing, reversing, and/or halting the progression of PD.
40. The method of embodiment 39, wherein the comparing the activation level of the one or more canonical pathways at the one or more time points following initiation of treatment to the baseline activation level indicates a likelihood of slowing, reversing, and/or halting the progression of PD in the subject.
41. The method of embodiment 39 or embodiment 40, wherein if the activation level of the one or more canonical pathways at the one or more time points following initiation of treatment to the baseline activation level increased, the comparison indicates an increased likelihood of slowing, reversing, and/or halting the progression of PD in the subject.
42. The method of any one of embodiments 39-41, wherein an increased activation level of the one or more canonical pathways at the one or more time points following initiation of treatment as compared to the baseline activation level indicates an increase in likelihood of slowing, reversing, and/or halting the progression of PD in the subject.
43. The method of any one of embodiments 39-42, wherein the activation level of the one or more canonical pathways at the one or more time points following initiation of treatment as compared to the baseline activation level increased.
44. The method of any one of embodiments 39-43, wherein the activation level of at least a threshold percentage of the one or more canonical pathways have increased in the biological sample at the one or more time points following initiation of the therapy compared to the baseline activation level.
45. The method of embodiment 44, wherein the threshold percentage is at least 50%, at least 55%, at least 60%, at least 65%, at least 70%, at least 75%, at least 80%, at least 85%, at least 90%, or at least 95%.
46. The method of any one of embodiments 39-45, wherein the comparison indicates an increase in likelihood of slowing, reversing, and/or halting the progression of PD in the subject if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.
47. A method for treating Parkinson's Disease (PD) in a subject, the method comprising: a) determining a baseline expression level of NF-kB and/or calcineurin in a biological sample from a subject having PD; b) administering a therapeutic agent for the treatment of PD to the subject; c) determining an expression level of NF-kB and/or calcineurin in a biological sample from the subject at one or more time points following initiation of the treatment; and d) determining if the expression level of NF-kB and/or calcineurin at the one or more time points following initiation of the treatment decreased compared to the baseline expression level, wherein a decrease in the expression level of NF-kB and/or calcineurin at the one or more time points following initiation of the treatment decreased compared to the baseline expression level indicates an increased likelihood of therapeutic efficacy in treating PD in the subject.
48. A method for treating Parkinson's Disease (PD) in a subject, the method comprising: a) determining a baseline expression level of NF-kB and/or calcineurin in a biological sample from a subject having PD; b) administering a therapeutic agent for the treatment of PD to the subject; c) determining an expression level of NF-kB and/or calcineurin in a biological sample from the subject at one or more time points following initiation of the treatment; and d) comparing the expression level of NF-kB and/or calcineurin at the one or more time points following initiation of the treatment to the baseline expression level, wherein the comparison indicates a likelihood of therapeutic efficacy in treating PD in the subject.
49. The method of embodiment 48, wherein the comparison indicates that the expression level of NF-kB and/or calcineurin at the one or more time points following initiation of treatment decreased compared to the baseline expression level.
50. The method of embodiment 48, wherein if the expression level of NF-kB and/or calcineurin at the one or more time points following initiation of treatment decreased compared to the baseline expression level, the comparison indicates an increased likelihood of therapeutic efficacy for the treatment of PD in the subject and/or indicates an increased degree of effectiveness at treating PD in the subject.
51. The method of embodiment 48, wherein if the expression level of NF-kB and/or calcineurin at the one or more time points following initiation of treatment did not decrease compared to the baseline expression level, the comparison indicates a neutral or decreased likelihood of therapeutic efficacy for the treatment of PD in the subject and/or indicates a neutral or decreased degree of effectiveness at treating PD in the subject.
52. The method of embodiment 48, wherein a decreased expression level of NF-kB and/or calcineurin at the one or more time points following initiation of the treatment compared to the baseline expression level indicates an increase in likelihood of therapeutic efficacy in treating PD in the subject.
53. The method of any one of embodiments 47-52, wherein the baseline expression level is determined at or at least 1 day, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment.
54. The method of any one of embodiments 47-53, wherein the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment.
55. The method of any one of embodiments 47-54, wherein the one or more time points following initiation of treatment with the therapeutic agent comprises a time point at or about 2 months following initiation of treatment.
56. The method of any one of embodiments 47-55, wherein the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.
57. The method of any one of embodiments 47-56, wherein the expression level of NF-kB and/or calcineurin is determined by an assay.
58. The method of embodiment 57, wherein the assay is selected from the group consisting of mass spectrometry, enzyme-linked immunosorbent assay (ELISA), western blotting, or polymerase chain reaction (PCR).
59. The method of embodiment 58, wherein the PCR is real-time PCR.
60. The method of any one of embodiments 47-59, wherein the expression level of NF-kB and/or calcineurin is decreased at the one or more time points following initiation of treatment compared to the baseline expression level.
61. The method of any one of embodiments 47-60, further comprising continuing treatment if the comparison indicates an increased likelihood of therapeutic efficacy.
62. The method of any one of embodiments 47-61, further comprising continuing treatment if the expression level of NF-kB and/or calcineurin at the one or more time points following initiation of the treatment decreased compared to the baseline expression level.
63. The method of any one of embodiments 47-62, further comprising discontinuing treatment if the expression level of NF-kB and/or calcineurin at the one or more time points following initiation of the treatment did not decrease compared to the baseline expression level.
64. The method of any one of embodiments 47-63, wherein: (i) treatment is continued if the expression level of NF-kB and/or calcineurin have decreased in the biological sample at the one or more time points following initiation of the therapy compared to the baseline expression level; or (ii) treatment is discontinued or altered if the expression level of NF-kB and/or calcineurin have not decreased in the biological sample at the one or more time points following initiation of the therapy compared to the baseline expression level.
65. The method of any one of embodiments 1-14, 23-38, and 47-64, wherein determining the baseline expression level of NF-kB and/or calcineurin occurs prior to administering the therapy. 66. A method for treating Parkinson's Disease (PD) in a subject, the method comprising: a) determining a baseline activation level of one or more canonical pathways in a biological sample from a subject having PD, wherein the one or more canonical pathways are associated with cellular immune response signaling, neuroinflammation signaling, or PD signaling pathways; b) administering a therapeutic agent for the treatment of PD to the subject; c) determining an activation level of one or more canonical pathways in a biological sample from the subject at one or more time points following initiation of the treatment; and d) comparing the activation level of the one or more canonical pathways at the one or more time points following initiation of treatment to the baseline expression level, wherein an increase in the activation level of the one or more canonical pathways indicates an increased likelihood of therapeutic efficacy.
67. The method of embodiment 66, further comprising continuing treatment if the activation level of the one or more canonical pathways indicates an increased likelihood of therapeutic efficacy.
68. The method of embodiment 66 or embodiment 67, wherein: (i) treatment is continued if the activation level of the one or more canonical pathways have increased in the biological sample at the one or more time points following initiation of the therapy compared to the baseline activation level; or (ii) treatment is discontinued or altered if the activation level of the one or more canonical pathways have not increased in the biological sample at the one or more time points following initiation of the therapy compared to the baseline activation level.
69. The method of any one of embodiments 66-68, wherein treatment is continued if the activation level of at least a threshold percentage of the one or more canonical pathways have increased in the biological sample at the one or more time points following initiation of the therapy compared to the baseline activation level.
70. The method of any one of embodiments 66-69, wherein the activation level of at least a threshold percentage of the one or more canonical pathways have increased in the biological sample at the one or more time points following initiation of the therapy compared to the baseline activation level.
71. The method of embodiment 69 or embodiment 70, wherein the threshold percentage is at least 50%, at least 55%, at least 60%, at least 65%, at least 70%, at least 75%, at least 80%, at least 85%, at least 90%, or at least 95%.
72. The method of any one of embodiments 66-71, wherein treatment is continued if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.
73. The method of any one of embodiments 66-72, wherein the activation level of the one or more canonical pathways indicates an increased likelihood of therapeutic efficacy if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.
74. The method of any one of embodiments 15-22, 39-46, and 66-73, wherein the one or more canonical pathways comprises a pathway associated with cellular immune response signaling, neuroinflammation signaling, and/or PD signaling, or any combination thereof.
75. The method of any one of embodiments 15-22, 39-46, and 66-74, wherein the one or more canonical pathways are selected from the group consisting of IL-8 Signaling, fMLP Signaling in Neutrophils, Role of NFAT in Regulation of the Immune Response, Fcγ Receptor-mediated Phagocytosis in Macrophages and Monocytes, CXCR4 Signaling, PKCθ Signaling in T Lymphocytes, Leukocyte Extravasation Signaling, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, CCR3 Signaling in Eosinophils, IL-3 Signaling, GM-CSF Signaling, Macropinocytosis Signaling, IL-7 Signaling Pathway, Interferon Signaling, Nur77 Signaling in T Lymphocytes, IL-9 Signaling, Antiproliferative Role of TOB in T Cell Signaling, CCR5 Signaling in Macrophages, Role of PKR in Interferon Induction and Antiviral Response, Production of Nitric Oxide and Reactive Oxygen Species in Macrophages, and iNOS Signaling.
76. The method of any one of embodiments 15-22, 39-46, and 66-75, wherein the one or more canonical pathways are selected from the group consisting of fMLP Signaling in Neutrophils, Role of NFAT in Regulation of the Immune Response, PKCθ Signaling in T Lymphocytes, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, IL-3 Signaling, GM-CSF Signaling, Nur77 Signaling in T Lymphocytes, IL-9 Signaling, Role of PKR in Interferon Induction and Antiviral Response, Production of Nitric Oxide and Reactive Oxygen Species in Macrophages, and iNOS Signaling.
77. The method of any one of embodiments 1-76, wherein the therapeutic agent is selected from the group consisting of granulocyte macrophage colony stimulating factor (GM-CSF) or an analog thereof, GM-CSF mRNA, vasoactive intestinal peptide (VIP) or an analog thereof, and VIP mRNA.
78. The method of any one of embodiments 1-76, wherein the therapeutic agent is an agonist of granulocyte-macrophage colony stimulating factor 2 receptor (CSF2R) or vasoactive intestinal peptide receptor 2 (VIP2R).
79. The method of embodiment 78, wherein the agonist is a peptide or peptide-like agonist of CSF2R or VIP2R.
80. The method of embodiment 78, wherein the agonist is an antibody or a fragment thereof that binds to CSF2R or VIP2R.
81. The method of any one of embodiments 1-76, wherein the therapeutic agent is a mimetic of GM-CSF or is a mimetic of VIP.
82. The method of any one of embodiments 1-76, wherein the therapeutic agent is levodopa.
83. The method of any one of embodiments 1-76, wherein the therapeutic agent is a dopamine receptor agonist.
84. The method of embodiment 77, wherein the GM-CSF or analog thereof is sargramostim.
85. The method of embodiment 84, wherein the sargramostim is administered subcutaneously five times per week.
86. The method of embodiment 77, wherein the GM-CSF or analog thereof is molgramostim.
87. The method of embodiment 77, wherein the VIP or analog thereof is aviptadil.
88. The method of embodiment 77, wherein the GM-CSF mRNA encodes sargramostim or molgramostim.
89. The method of embodiment 77, wherein the VIP mRNA encodes aviptadil.
90. The method of any one of embodiments 1-76, wherein the therapeutic agent is an agent that reacts with and/or induces molecules that react with a GM-CSF receptor or a VIP receptor.
91. The method of embodiment 90, wherein the GM-CSF receptor is CSF2R and/or the VIP receptor is VIP2R.
92. The method of any one of embodiments 1-76, wherein the therapeutic agent is an immunogen that induces a humoral immune response against at least one abnormal protein of PD.
93. The method of embodiment 92, wherein the immunogen is nitrated alpha synuclein or a fragment thereof.
94. The method of embodiment 93, wherein the immunogen is a nitrated alpha synuclein fragment, and wherein the nitrated alpha synuclein fragment is a carboxy terminal fragment consisting of the carboxy terminal 20 amino acids of alpha synuclein up to the carboxy terminal 70 amino acids of alpha synuclein.
95. The method of any one of embodiments 92-94, wherein the immunogen is human nitrated alpha synuclein or a fragment thereof.
96. The method of any one of embodiments 92-95, wherein the immunogen is comprised in a composition that further comprises at least one adjuvant that stimulates regulatory T cells.
97. The method of embodiment 96, wherein the adjuvant is selected from the group consisting of VIP, vitamin D, GM-CSF, and transforming growth factor beta (TGFβ).
98. The method of any one of embodiments 93-97, wherein the nitrated alpha synuclein comprises the amino acid sequence of SEQ ID NO: 4, or comprises an amino acid sequence having at least 90%, 91%, 92%, 93%, 94%, 95%, 96%, 97%, 98%, or 99% sequence identity to the amino acid sequence of SEQ ID NO: 4.
99. The method of any one of embodiments 93-97, wherein the nitrated alpha synuclein fragment comprises amino acid residues 101-140 of SEQ ID NO: 4, or comprises an amino acid sequence having at least 90%, 91%, 92%, 93%, 94%, 95%, 96%, 97%, 98%, or 99% sequence identity to amino acid residues 101-140 of the amino acid sequence of SEQ ID NO: 4.
100. The method of any one of embodiments 96-99, wherein the composition further comprises a pharmaceutically acceptable carrier.
101. The method of any one of embodiments 1-100, wherein the biological sample comprises peripheral blood lymphocytes.
102. The method of any one of embodiments 1-101, wherein the biological sample comprises T cells.
103. The method of any one of embodiments 1-102, wherein the biological sample is obtained from the subject by leukapheresis.
104. A composition for detecting NF-kB and/or calcineurin expression in a biological sample, wherein the composition comprises a kit for a mass spectrometry analysis, an ELISA assay, a western blotting assay, or a PCR reaction.
105. The composition of embodiment 104, wherein the kit comprises a reagent for an ELISA assay.
106. The composition of embodiment 105, wherein the kit for the ELISA assay comprises a multi-well sample plate that is coated with immobilized capture antibodies that bind to NF-kB and/or calcineurin; detection antibodies covalently linked to an enzyme wherein the detection antibodies also bind to NF-kB and/or calcineurin; a colored or fluorescent product that will be catalyzed by the enzyme attached to the detection antibody; and appropriate buffers.
107 Biomarkers for monitoring the progression of Parkinson's Disease (PD) and/or the effectiveness of therapeutics for the treatment of PD, wherein the biomarkers are NF-κB and calcineurin; or are one or more canonical pathways associated with cellular immune response signaling, neuroinflammation signaling, or PD signaling pathways.
108. The method of embodiment 107, wherein the one or more canonical pathways are selected from the group consisting of IL-8 Signaling, fMLP Signaling in Neutrophils, Role of NFAT in Regulation of the Immune Response, Fcγ Receptor-mediated Phagocytosis in Macrophages and Monocytes, CXCR4 Signaling, PKCθ Signaling in T Lymphocytes, Leukocyte Extravasation Signaling, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, CCR3 Signaling in Eosinophils, IL-3 Signaling, GM-CSF Signaling, Macropinocytosis Signaling, IL-7 Signaling Pathway, Interferon Signaling, Nur77 Signaling in T Lymphocytes, IL-9 Signaling, Antiproliferative Role of TOB in T Cell Signaling, CCR5 Signaling in Macrophages, Role of PKR in Interferon Induction and Antiviral Response, Production of Nitric Oxide and Reactive Oxygen Species in Macrophages, and iNOS Signaling.
109 The method of embodiment 107 or embodiment 108, wherein the one or more canonical pathways are selected from the group consisting of fMLP Signaling in Neutrophils, Role of NFAT in Regulation of the Immune Response, PKCθ Signaling in T Lymphocytes, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, IL-3 Signaling, GM-CSF Signaling, Nur77 Signaling in T Lymphocytes, IL-9 Signaling, Role of PKR in Interferon Induction and Antiviral Response, Production of Nitric Oxide and Reactive Oxygen Species in Macrophages, and iNOS Signaling.
110. A therapeutic agent for use in the treatment of Parkinson's Disease (PD) in a subject, wherein the therapeutic agent is for use in a method comprising: a) determining a baseline expression level of NF-kB and/or calcineurin in a biological sample from a subject having PD; b) determining an expression level of NF-kB and/or calcineurin in a biological sample from the subject at one or more time points following administration of the treatment; and c) comparing the expression level of NF-kB and/or calcineurin at the one or more time points following initiation of the treatment to the baseline expression level, wherein the comparison indicates a likelihood of therapeutic efficacy in treating PD in the subject.
11. Use of a therapeutic agent in the treatment of Parkinson's Disease (PD) in a subject, wherein the use comprises: a) determining a baseline expression level of NF-kB and/or calcineurin in a biological sample from a subject having PD; b) determining an expression level of NF-kB and/or calcineurin in a biological sample from the subject at one or more time points following administration of the therapeutic agent; and c) comparing the expression level of NF-kB and/or calcineurin at the one or more time points following initiation of the treatment to the baseline expression level, wherein the comparison indicates a likelihood of therapeutic efficacy in treating PD in the subject.
112. A therapeutic agent for use in the treatment of Parkinson's Disease (PD) in a subject, wherein the therapeutic agent is for use in a method comprising: a) determining a baseline activation level of one or more canonical pathways in a biological sample from a subject having PD, wherein the one or more canonical pathways are associated with cellular immune response signaling, neuroinflammation signaling, or PD signaling pathways; b) determining an activation level of one or more canonical pathways in a biological sample from the subject at one or more time points following administration of the treatment; and c) comparing the activation level of one or more canonical pathways in a biological sample from the subject at the one or more time points following initiation of the treatment to the baseline expression level, wherein the comparison indicates a likelihood of therapeutic efficacy in treating PD in the subject.
113. Use of a therapeutic agent in the treatment of Parkinson's Disease (PD) in a subject, wherein the use comprises: a) determining a baseline activation level of one or more canonical pathways in a biological sample from a subject having PD, wherein the one or more canonical pathways are associated with cellular immune response signaling, neuroinflammation signaling, or PD signaling pathways; b) determining an activation level of one or more canonical pathways in a biological sample from the subject at one or more time points following administration of the treatment; and c) comparing the activation level of one or more canonical pathways in the biological sample from the subject at the one or more time points following initiation of the treatment to the baseline expression level, wherein the comparison indicates a likelihood of therapeutic efficacy in treating PD in the subject.

### IV. Examples

The following examples are included for illustrative purposes only and are not intended to limit the scope of the invention.

### Example 1 Administration of Sargramostim for the Treatment of Parkinson's Disease

### Study Design and Subject Enrollment

The study is an unblinded, open-label, single-center phase 1 clinical trial performed at the University of Nebraska Medical Center (UNMC), Omaha, NE, USA designed to test safety, tolerability, and biomarker discovery utilizing a 3 μg/kg/day (5 days on 2 days off) sargramostim regimen. In total, 6 PD subjects were enrolled, and of those, 5 met study entry criteria. All were recruited from the Omaha metropolitan area, assessed for three months for clinical status and baseline immune, hematological, and metabolic profiling, and treated for 12 months between January 2019 to July 2020. Eligibility criteria were 35 to 85 years of age with PD signs and symptoms that included asymmetric bradykinesia, resting tremor, and/or muscle rigidity persisting for longer than three years with less than stage four on the Hoehn and Yahr disease scale. Exclusion criteria included poor venous access, inability to undergo leukapheresis, use of a wheelchair, walker, and/or cane, multiple system atrophy, corticobasal degeneration, unilateral Parkinsonism of >3 years, prior head injury, stroke, brain surgery including deep brain stimulation, a family history of >1 blood relative with PD, mental illness, cognitive impairment, autoimmune, systemic inflammatory or hematologic diseases, current treatment with neuroleptics or lithium, past treatment with sargramostim, past immunosuppressive treatments, and known allergies to colony-stimulating factors or yeast-derived products.

TABLE 1
Complete blood count (CBC) for subjects before and during sargramostim treatment

| | | Baseline | Months after samgramostim initiation (number of subjects) | | | | | | | |
|---|---|---|---|---|---|---|---|---|---|---|
| | | Mo −4-0 | 1 | 2 | 3 | 4 | 5 | 6 | 8 | 10 |
| Variable | | ( [symbol] ) | ( [symbol] ) | ( [symbol] ) | (5) | (4) | (6) | (5) | (5) | (5) |
| VVBC × 10³/μL | Mean | 5.50 | 7.92 | 14.46 | 7.50 | 7.80 | 7.96 | 14.56 | 7.47 | 7.48 |
| | SD | 1.35 | 1.80 | 2.24 | 1.15 | 1.25 | 0.75 | 6.70 | 1.64 | 1.22 |
| RBC × 10⁶/μL | Mean | 4.74 | 5.01 | 4.81 | 5.06 | 6.11 | 4.97 | 4.84 | 5.01 | 6.02 |
| | SD | 0.39 | 0.27 | 0.24 | 0.29 | 0.26 | 0.19 | 0.29 | 0.31 | 0.20 |
| Hemoglobin, g/dL | Mean | 15.07 | 15.34 | 14.90 | 15.42 | 15.48 | 14.98 | 14.54 | 15.04 | 15.00 |
| | SD | 0.84 | 0.89 | 0.57 | 0.90 | 0.88 | 0.72 | 0.88 | 1.00 | 0.72 |
| Hematocrit, % | Mean | 45.70 | 46.70 | 45.38 | 46.80 | 47.70 | 46.04 | 44.98 | 48.34 | 48.46 |
| | SD | 2.65 | 3.16 | 2.61 | 2.79 | 3.24 | 2.10 | 2.88 | 3.13 | 2.38 |
| MCV^{d}, fL | Mean | 93.71 | 93.2 | 94.44 | 92.54 | 93.3 | 92.68 | 92.98 | 92.57 | 92.48 |
| | SD | 2.69 | 2.38 | 2.82 | 2.29 | 2.33 | 2.19 | 2.22 | 3.19 | 2.88 |
| MCHC^{d}, % | Mean | 32.98 | 32.86 | 32.88 | 32.94 | 32.48 | 32.62 | 32.34 | 32.48 | 32.32 |
| | SD | 0.85 | 0.71 | 0.64 | 0.33 | 0.57 | 0.40 | 0.58 | 0.58 | 0.47 |
| RBC Distribution Width, % | Mean | 13.02 | 13.18 | 13.22 | 13.00 | 12.98 | 13.20 | 13.54 | 13.66 | 13.44 |
| | SD | 0.41 | 0.22 | 0.43 | 0.34 | 0.49 | 0.42 | 0.53 | 0.69 | 0.59 |
| Platlets × 10³/μL | Mean | 22 [symbol] .76 | 205.40 | 218.20 | 220.80 | 212.00 | 217.60 | 223.80 | 221.80 | 228.00 |
| | SD | 49.25 | 32.23 | 44.23 | 58.20 | 38.32 | 42.33 | 60.47 | 34.50 | 40.99 |
| Neutrophils, % | Mean | 63.05 | 64.20 | 63.40 | 64.80 | 66.25 | 63.00 | 57.80 | 63.90 | 61.40 |
| | SD | 6.81 | 2.17 | 5.22 | 8.84 | 6.13 | 4.90 | 7.09 | 1.60 | 8.50 |
| Lymphocytes, % | Mean | 24.29 | 19.40 | 9.60 | 20.20 | 20.00 | 19.80 | 17.00 | 19.30 | 20.20 |
| | SD | 8.18 | 2.79 | 2.30 | 6.83 | 6.38 | 8.42 | 8.43 | 3.53 | 6.30 |
| Monocytes, % | Mean | 8.71 | 8.60 | 2.80 | 6.60 | 6.00 | 8.60 | 6.80 | 8.10 | 9.40 |
| | SD | 3.52 | 0.89 | 2.69 | 1.52 | 0.82 | 3.13 | 2.51 | 2.75 | 3.97 |
| Eosinophils, % | Mean | 2.76 | 8.00 | 21.00 | 6.80 | 6.25 | 7.00 | 14.60 | 7.20 | 7.60 |
| | SD | 0.89 | 1.41 | 5.66 | 3.56 | 2.22 | 2.45 | 8.20 | 3.11 | 4.62 |
| Basophils, % | Mean | 1.00 | 1.00 | 0.20 | 1.00 | 0.75 | 1.00 | 1.80 | 1.00 | 1.00 |
| | SD | 0.45 | 0.00 | 0.45 | 0.00 | 0.50 | 0.00 | 2.95 | 0.00 | 0.00 |
| Neutrophils × 10³/μL | Mean | 3.45 | 5.10 | 9.66 | 4.88 | 5.23 | 5.02 | 9.66 | 4.81 | 4.80 |
| | SD | 0.98 | 1.13 | 2.21 | 0.90 | 1.22 | 0.54 | 4.95 | 1.03 | 0.82 |
| Lymphocytes × 10³/μL | Mean | 1.35 | 1.54 | 1.42 | 1.52 | 1.53 | 1.58 | 1.66 | 1.43 | 1.52 |
| | SD | 0.51 | 0.50 | 0.52 | 0.53 | 0.43 | 0.44 | 0.61 | 0.37 | 0.55 |
| Monocytes × 10³/μL | Mean | 0.48 | 0.52 | 0.40 | 0.50 | 0.45 | 0.72 | 0.92 | 0.68 | 0.86 |
| | SD | 0.14 | 0.11 | 0.39 | 0.14 | 0.10 | 0.33 | 0.53 | 0.08 | 0.23 |
| Eosinophils × 10³/μL | Mean | 0.18 | 0.82 | 2.46 | 0.52 | 0.48 | 0.54 | 2.28 | 0.63 | 0.58 |
| | SD | 0.07 | 0.24 | 0.98 | 0.33 | 0.24 | 0.23 | 2.26 | 0.32 | 0.39 |
| Basophils × 10³/μL | Mean | 0.05 | 0.08 | 0.02 | 0.10 | 0.08 | 0.08 | 0.04 | 0.10 | 0.10 |
| | SD | 0.05 | 0.04 | 0.04 | 0.00 | 0.05 | 0.04 | 0.05 | 0.00 | 0.00 |

| | | Months after samgramostim initiation (number of subjects) | | | |
|---|---|---|---|---|---|
| | | 12 | Mo 1-12 | p values^{a} | |
| Variable | | (4) | (4- [symbol] ) | ANOVA^{b} | t-test^{c} |
| VVBC × 10³/μL | Mean | 8.75 | 9.37 | <0.0001 | 0.0002 |
| | SD | 1.88 | 3.79 | | |
| RBC × 10⁶/μL | Mean | 4.58 | 4.94 | 0.7465 | 0.1615 |
| | SD | 0.87 | 0.36 | | |
| Hemoglobin, g/dL | Mean | 13.55 | 14.93 | 0.3080 | 0.5785 |
| | SD | 2.62 | 1.13 | | |
| Hematocrit, % | Mean | 41.93 | 45.88 | 0.5018 | 0.8 [symbol] |
| | SD | 8.29 | 3.59 | | |
| MCV^{d}, fL | Mean | 91.55 | 92.88 | 0.8811 | 0.2357 |
| | SD | 3.28 | 2.48 | | |
| MCHC^{d}, % | Mean | 32.33 | 32.58 | 0.2078 | 0.0366 |
| | SD | 0.98 | 0.59 | | |
| RBC Distribution Width, % | Mean | 13.45 | 13.30 | 0.1102 | 0.0521 |
| | SD | 0.62 | 0.49 | | |
| Platlets × 10³/μL | Mean | 222.00 | 218.91 | 0.9988 | 0.5385 |
| | SD | 23.58 | 39.83 | | |
| Neutrophils, % | Mean | 62.50 | 62.97 | 0.7701 | 0.7279 |
| | SD | 5.20 | 5.88 | | |
| Lymphocytes, % | Mean | 17.80 | 18.08 | 0.0048 | 0.0014 |
| | SD | 7.68 | 6.27 | | |
| Monocytes, % | Mean | 7.25 | 6.90 | 0.0213 | 0.0521 |
| | SD | 2.50 | 2.93 | | |
| Eosinophils, % | Mean | 10.00 | 9.91 | <0.0001 | <0.0001 |
| | SD | 7.79 | 6.46 | | |
| Basophils, % | Mean | 0.75 | 0.95 | 0.4528 | 0.6909 |
| | SD | 0.60 | 1.02 | | |
| Neutrophils × 10³/μL | Mean | 5.50 | 8.08 | <0.0001 | 0.0003 |
| | SD | 1.60 | 2.74 | | |
| Lymphocytes × 10³/μL | Mean | 1.45 | 1.52 | 0.9807 | 0.2232 |
| | SD | 0.53 | 0.46 | | |
| Monocytes × 10³/μL | Mean | 0.60 | 0.60 | 0.0216 | 0.0 [symbol] |
| | SD | 0.22 | 0.30 | | |
| Eosinophils × 10³/μL | Mean | 1.10 | 1.02 | <0.0001 | 0.002 [symbol] |
| | SD | 0.74 | 1.12 | | |
| Basophils × 10³/μL | Mean | 0.08 | 0.07 | 0.0239 | 0.052 [symbol] |
| | SD | 0.05 | 0.04 | | |

^{a}Signficant variable values and p values ≤0.05 are emboldened.
^{b}Comparison of means from baseline (Mo −4-0) vs sargramostim treatment at each month; Dunnett's post hoc test.
^{c}Comparison of means from baseline (Mo −4-0) vs combined months (1-12) on sargramastim and adjusted for false discovery rate.
^{d}MCV. Mean Corpuscular Volume; MCHC, Mean Corpuscular Hemoglobulin Concentration [symbol]
indicates data missing or illegible when filed

### Procedure

The current study (Phase 1b) was designed for safety and tolerability assessment for direct comparison with a previously published (Phase 1a) study in which PD subjects self-administered sargramostim at 6 μg/kg/day for two months (NCT01882010). For the current study, PD subjects underwent three pre-treatment monthly interval appointments to determine baseline immune, hematologic, and metabolic profiles (Tables 1-3, baseline column). On visit three (month 0), subjects initiated self-administered sargramostim at 3 μg/kg/day (five days on, two days off) subcutaneously for 12 months, returning for clinical assessments every four weeks.

TABLE 2
T cell frequency and number from subjects before and during treatment with sargramostim

| | | Baseline | Month after sargramostim initiatic (number of subjects) | | | | | | | | | | | |
|---|---|---|---|---|---|---|---|---|---|---|---|---|---|---|
| | | Mo −4-0 | 1 | 2 | 3 | 4 | 5 | 6 | 8 | 10 | 12 | 1-12 | p value^{a} | |
| Variable | | (5) | (5) | (4) | (5) | (4) | (5) | (5) | (5) | (5) | (4) | (5) | ANOVA^{b} | t-test^{c} |
| Percentage | | | | | | | | | | | | | | |
| CD3+ | Mean | 67.0 | 70.3 | 74.0 | 73.4 | 72.9 | 72.2 | 72.4 | 71.9 | 72.3 | 72.0 | 72.3 | 0.2596 | 0.0063 |
| | SD | 5.9 | 5.2 | 5.5 | 6.0 | 7.0 | 5.4 | 6.2 | 8.6 | 5.4 | 3.9 | 5.5 | | |
| CD4+ | Mean | 46.7 | 49.3 | 50.2 | 50.2 | 48.7 | 49.7 | 49.3 | 49.8 | 50.0 | 46.7 | 49.4 | 0.9969 | 0.2555 |
| | SD | 6.8 | 9.0 | 9.2 | 12.9 | 14.5 | 12.9 | 5.2 | 12.7 | 10.9 | 8.6 | 9.9 | | |
| CD8+ | Mean | 17.5 | 19.2 | 22.1 | 21.6 | 22.7 | 20.9 | 21.3 | 20.8 | 20.4 | 23.8 | 21.3 | 0.9709 | 0.1636 |
| | SD | 9.8 | 8.7 | 10.2 | 10.2 | 10.9 | 3.3 | 10.3 | 8.7 | 9.1 | 11.3 | 8.9 | | |
| Number/ [symbol] L | | | | | | | | | | | | | | |
| CD3+ | Mean | 940 | 1089 | 1262 | 1164 | 1219 | 1124 | 1053 | 1088 | 1260 | 1178 | 1155 | 0.5775 | 0.0433 |
| | SO | 334 | 377 | 221 | 356 | 375 | 365 | 300 | 261 | 437 | 198 | 309 | | |
| CD4+ | Mean | 636 | 743 | 872 | 754 | 769 | 742 | 732 | 738 | 841 | 765 | 771 | 0.6482 | 0.0433 |
| | SD | 206 | 231 | 293 | 141 | 175 | 235 | 275 | 206 | 263 | 188 | 210 | | |
| CD8+ | Mean | 282 | 317 | 366 | 381 | 419 | 355 | 296 | 332 | 385 | 389 | 358 | 0.9691 | 0.1916 |
| | SD | 189 | 234 | 159 | 287 | 302 | 262 | 140 | 193 | 280 | 212 | 216 | | |
| Ratio | | | | | | | | | | | | | | |
| CD4+/CD8+ | Mean | 4.4 | 3.2 | 2.9 | 3.1 | 3.0 | 3.1 | 3.0 | 3.0 | 3.2 | 2.7 | 3.0 | 0.9421 | 0.0905 |
| | SD | 3.7 | 2.0 | 2.0 | 2.1 | 2.4 | 2.1 | 2.1 | 1.8 | 2.3 | 2.3 | 1.9 | | |

^{a}Signficant variable values and p values ≤ 0.05 are emboldened.
^{b}Comparison of means from baseline vs sargramostim treatment at each month; Dunnett's post hoc test.
^{c}Comparison of means from baseline (Mo −4-0) vs combined months (1-12) on sargramostim and adjusted for false discovery rate. [symbol]
indicates data missing or illegible when filed

Prior to treatment initiation and at two and six months after initiation, subjects underwent leukapheresis to allow for peripheral blood lymphocyte enrichment to obtain sufficient numbers of CD4+ Treg to complete analyses for DNA methylation assessment, flow cytometric analysis, and Treg functional assessments. Peripheral blood samples, physical examinations, and MDS-UPDRS Parts I-IV assessments were completed during each appointment. Anti-parkinsonian therapies (carbidopa-levodopa) were continued during the course of study without modification for 4/5 subjects (Table 4). The primary neurologist (PS) performed MDS-UPDRS III assessments in the “ON” state at the same time of day for each study visit. Observable and/or clinical adverse events discovered during physical examination were recorded directly by the study neurologist such as elevated white blood cell counts or site injection reactions. Subjects were also provided with an “adverse event log” for events occurring between visits. The severity of the adverse event and likelihood of relationship to treatment were determined by the study neurologist. Study drug was withheld during the drug holiday for two days prior to each clinical visit, except for during leukapheresis visits (month 2 and 6). On these visits, subjects did not undergo the two-day drug holiday, and blood was harvested on day five of treatment. WBC counts with differentials, immunocyte numbers, CD4 and CD8 T cell percentages and ratios, and comprehensive blood chemistry profiles were monitored for safety.

TABLE 3
Comprehensive metabolic panel before and during sargramostim treatment

| Variable | | Baseline | Months after sargramostim initiation (number of subjects) | | | | | | | |
|---|---|---|---|---|---|---|---|---|---|---|
| Reference | | Mo −4-0 | 1 | 2 | 3 | 4 | 5 | 6 | 8 | 10 |
| Values | | (5) | (5) | (5) | (5) | (4) | (5) | (5) | (5) | (5) |
| ALP | Mean | 76.19 | 70.20 | 67.60 | 68.00 | 65.50 | 67.80 | 67.40 | 67.00 | 66.00 |
| 32-91 U/L | SD | 27.25 | 22.39 | 23.74 | 21.53 | 15.95 | 24.99 | 25.48 | 25.45 | 17.68 |
| AST | Mean | 17.38 | 13.80 | 10.00 | 11.60 | 11.25 | 12.40 | 9.60 | 11.60 | 9.80 |
| 15-41 U/L | SD | 4.41 | 3.27 | 3.54 | 2.88 | 3.10 | 3.05 | 2.61 | 3.13 | 2.95 |
| ALT | Mean | 5.19 | 8.60 | 8.60 | 4.80 | 7.25 | 5.60 | 4.20 | 3.20 | 3.60 |
| 7-52 U/L | SD | 3.74 | 12.03 | 10.41 | 3.27 | 8.06 | 3.29 | 2.28 | 1.30 | 1.34 |
| Protein | Mean | 6.77 | 6.54 | 6.00 | 6.50 | 6.48 | 6.34 | 6.10 | 6.50 | 6.38 |
| 5.8-8.2 g/dL | SD | 0.26 | 0.32 | 0.25 | 0.31 | 0.46 | 0.29 | 0.21 | 0.22 | 0.37 |
| Albumin | Mean | 4.14 | 4.00 | 3.70 | 4.04 | 3.90 | 3.98 | 3.80 | 4.10 | 4.00 |
| 3.5-5.1 g/dL | SD | 0.22 | 0.21 | 0.21 | 0.30 | 0.26 | 0.26 | 0.19 | 0.00 | 0.23 |
| Bilirubin | Mean | 0.75 | 0.80 | 0.64 | 0.68 | 0.60 | 0.64 | 0.62 | 0.60 | 0.74 |
| 0.3-1.0 mg/dL | SD | 0.26 | 0.35 | 0.15 | 0.19 | 0.12 | 0.15 | 0.26 | 0.16 | 0.24 |
| Glucose | Mean | 92.76 | 96.20 | 92.80 | 86.20 | 111.50 | 90.80 | 98.40 | 86.60 | 90.60 |
| 70-139 mg/dL | SD | 12.65 | 8.07 | 31.33 | 11.86 | 66.74 | 12.76 | 14.15 | 10.78 | 12.03 |
| BUN | Mean | 22.71 | 22.40 | 22.00 | 20.40 | 19.25 | 22.60 | 21.80 | 24.60 | 22.60 |
| 8-20 mg/dL | SD | 8.18 | 7.09 | 4.36 | 4.98 | 7.37 | 5.86 | 3.70 | 8.26 | 6.19 |
| Creatinine | Mean | 1.02 | 1.00 | 1.03 | 0.96 | 0.88 | 0.91 | 1.00 | 0.99 | 0.99 |
| 0.64-1.27 mg/dL | SD | 0.23 | 0.26 | 0.24 | 0.21 | 0.14 | 0.26 | 0.25 | 0.28 | 0.20 |
| BUN/Creat Ratio | Mean | 23.02 | 23.24 | 21.84 | 21.74 | 22.13 | 25.76 | 22.62 | 25.04 | 23.00 |
| 10.0-20.0 | SD | 7.55 | 8.79 | 3.92 | 5.64 | 7.66 | 7.19 | 5.05 | 6.49 | 5.38 |
| GFR | Mean | 58.00 | 57.80 | 58.40 | 59.6 | 60.00 | 58.80 | 58.40 | 57.60 | 59.20 |
| >59 mL/min | SD | 3.99 | 4.92 | 3.58 | 0.89 | 0.00 | 2.68 | 3.58 | 5.37 | 1.79 |
| Calcium | Mean | 9.26 | 9.34 | 8.90 | 8.96 | 8.98 | 8.82 | 8.91 | 9.18 | 9.24 |
| 8.6-10.4 mg/DL | SD | 0.29 | 0.60 | 0.16 | 0.56 | 0.74 | 0.31 | 0.21 | 0.35 | 0.36 |
| Potassium | Mean | 4.03 | 3.94 | 3.86 | 4.16 | 4.05 | 4.04 | 3.78 | 4.12 | 3.92 |
| 3.5-5.1 mmol/L | SD | 0.32 | 0.27 | 0.43 | 0.31 | 0.29 | 0.24 | 0.30 | 0.38 | 0.37 |
| Sodium | Mean | 140.43 | 140.80 | 141.00 | 140.40 | 141.50 | 138.60 | 140.00 | 140.20 | 140.00 |
| 136-145 mmol/L | SD | 1.91 | 3.63 | 2.83 | 2.97 | 1.73 | 2.61 | 1.67 | 3.56 | 2.83 |
| Chloride | Mean | 104.05 | 104.20 | 104.20 | 104.40 | 105.25 | 104.80 | 104.20 | 103.40 | 103.80 |
| 98-107 mmol/L | SD | 2.67 | 4.87 | 3.11 | 4.39 | 1.71 | 4.44 | 2.59 | 3.21 | 2.77 |
| Anton Gap | Mean | 7.05 | 7.20 | 8.80 | 7.40 | 7.25 | 7.40 | 8.80 | 5.60 | 6.60 |
| 4-15 mmol/L | SD | 1.28 | 0.84 | 1.10 | 0.55 | 2.06 | 1.14 | 2.17 | 0.89 | 1.95 |
| Osmolality | Mean | 293.19 | 294.00 | 294.00 | 292.00 | 295.00 | 289.60 | 292.60 | 293.00 | 292.00 |
| 275-295 mOsm/kg | SD | 4.17 | 8.28 | 5.10 | 6.08 | 3.56 | 4.28 | 3.58 | 7.18 | 4.80 |
| CO2 | Mean | 29.33 | 29.40 | 28.00 | 28.60 | 29.00 | 26.40 | 27.00 | 31.20 | 29.60 |
| 22-32 mmol/L | SD | 1.65 | 1.95 | 1.41 | 1.82 | 3.56 | 2.07 | 1.22 | 1.30 | 2.07 |

| Variable | | Months after sargramostim initiation (number of subjects) | | | |
|---|---|---|---|---|---|
| Reference | | 12 | 1-12 | p value^{a} | |
| Values | | (4) | (5) | ANOVA^{b} | t-test^{c} |
| ALP | Mean | 71.25 | 67.84 | 0.992 | 0.4559 |
| 32-91 U/L | SD | 18.25 | 20.08 | | |
| AST | Mean | 9.25 | 11.07 | 0.0001 | <0.0001 |
| 15-41 U/L | SD | 5.91 | 3.40 | | |
| ALT | Mean | 4.75 | 5.60 | 0.7729 | 0.7654 |
| 7-52 U/L | SD | 2.06 | 5.99 | | |
| Protein | Mean | 6.18 | 6.33 | <0.0001 | <0.0001 |
| 5.8-8.2 g/dL | SD | 0.32 | 0.34 | | |
| Albumin | Mean | 3.85 | 3.93 | 0.0071 | 0.0055 |
| 3.5-5.1 g/dL | SD | 0.21 | 0.23 | | |
| Bilirubin | Mean | 0.68 | 0.67 | 0.8769 | 0.4641 |
| 0.3-1.0 mg/dL | SD | 0.39 | 0.22 | | |
| Glucose | Mean | 104.25 | 94.67 | 0.7970 | 0.7654 |
| 70-139 mg/dL | SD | 21.64 | 24.13 | | |
| BUN | Mean | 22.25 | 22.05 | 0.9609 | 0.7664 |
| 8-20 mg/dL | SD | 4.27 | 5.55 | | |
| Creatinine | Mean | 0.85 | 0.96 | 0.9206 | 0.5531 |
| 0.64-1.27 mg/dL | SD | 0.10 | 0.21 | | |
| BUN/Creat Ratio | Mean | 26.58 | 23.51 | 0.9752 | 0.7654 |
| 10.0-20.0 | SD | 6.11 | 5.97 | | |
| GFR | Mean | 60.25 | 58.84 | 0.9445 | 0.5686 |
| >59 mL/min | SD | 0.50 | 3.05 | | |
| Calcium | Mean | 9.33 | 9.07 | 0.1651 | 0.2574 |
| 8.6-10.4 mg/DL | SD | 0.50 | 0.43 | | |
| Potassium | Mean | 4.03 | 3.99 | 0.7403 | 0.7654 |
| 3.5-5.1 mmol/L | SD | 0.33 | 0.32 | | |
| Sodium | Mean | 142.50 | 140.49 | 0.6928 | 0.8616 |
| 136-145 mmol/L | SD | 3.32 | 2.80 | | |
| Chloride | Mean | 106.75 | 104.49 | 0.9420 | 0.7654 |
| 98-107 mmol/L | SD | 3.10 | 3.30 | | |
| Anton Gap | Mean | 7.75 | 7.42 | 0.0198 | 0.5666 |
| 4-15 mmol/L | SD | 1.26 | 1.67 | | |
| Osmolality | Mean | 297.75 | 293.19 | 0.6893 | 0.8727 |
| 275-295 mOsm/kg | SD | 5.32 | 5.47 | | |
| CO2 | Mean | 28.00 | 28.58 | 0.005 | 0.4559 |
| 22-32 mmol/L | SD | 0.82 | 2.22 | | |

^{a}Significant variable means and p vales ≤ 0.05 are emboldened.
^{b}Comparison of means from baseline (Mo −4-0) vs means from sargramostim treatment at each month; Dunnett's post hoc test.
^{c}Comparison of means from baseline (Mo −4-0) vs means from combined months (1-12) on sargramostim and adjusted for false discovery rate.

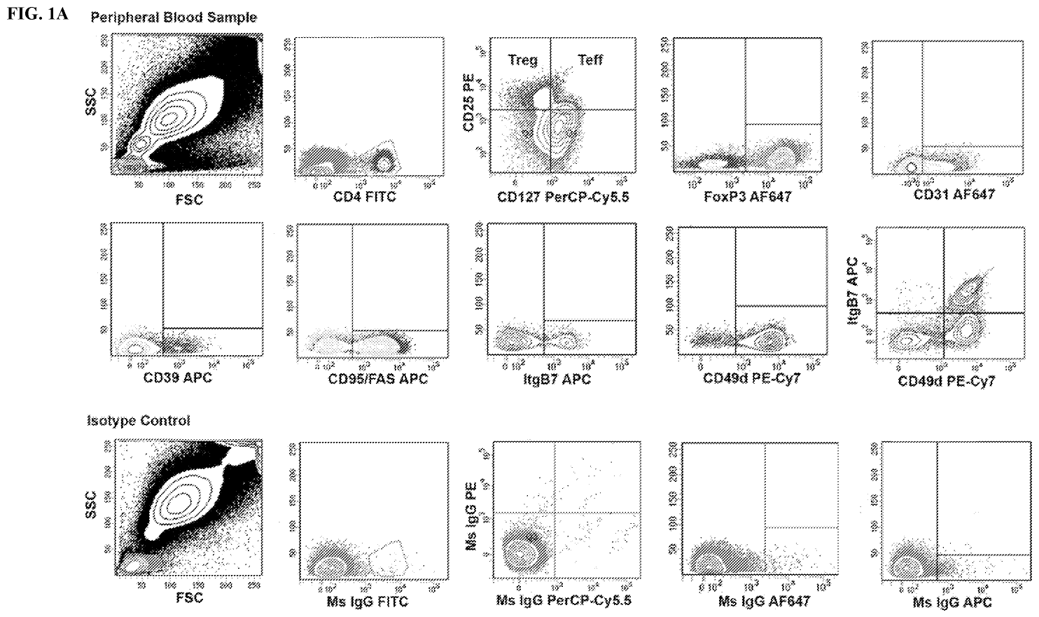

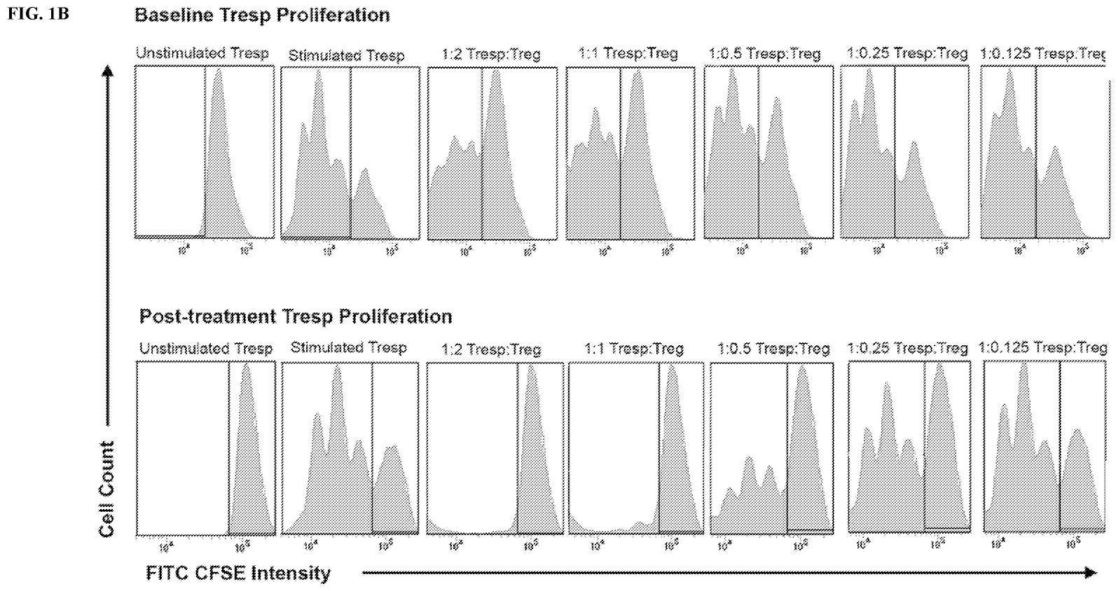

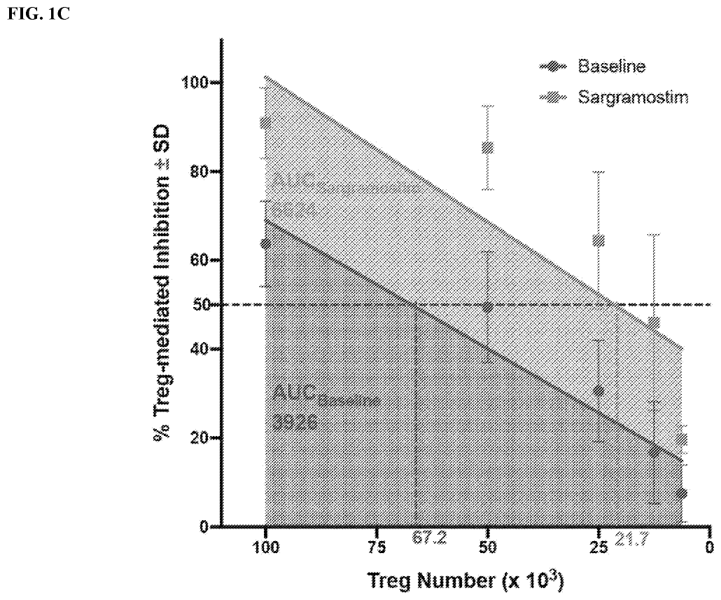

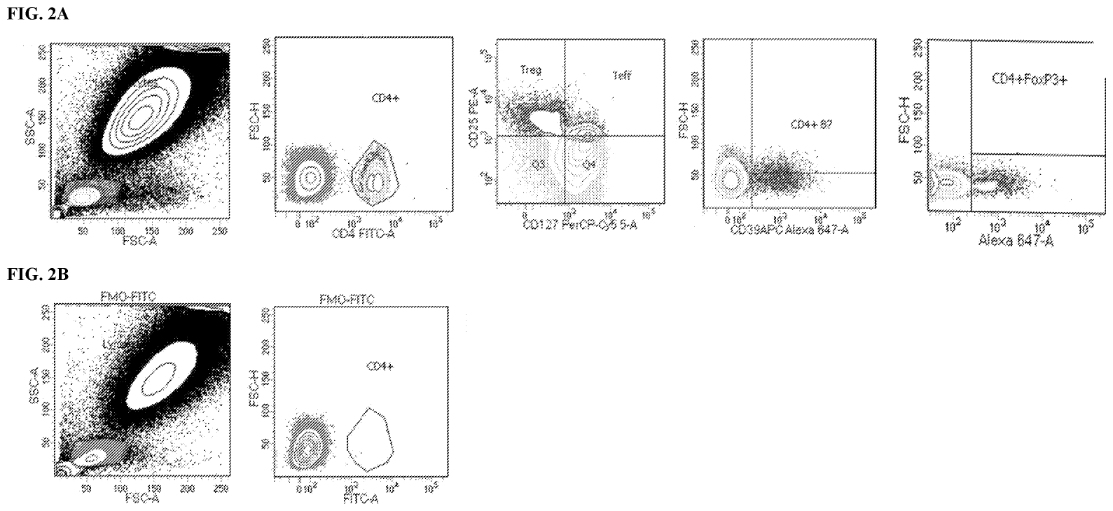

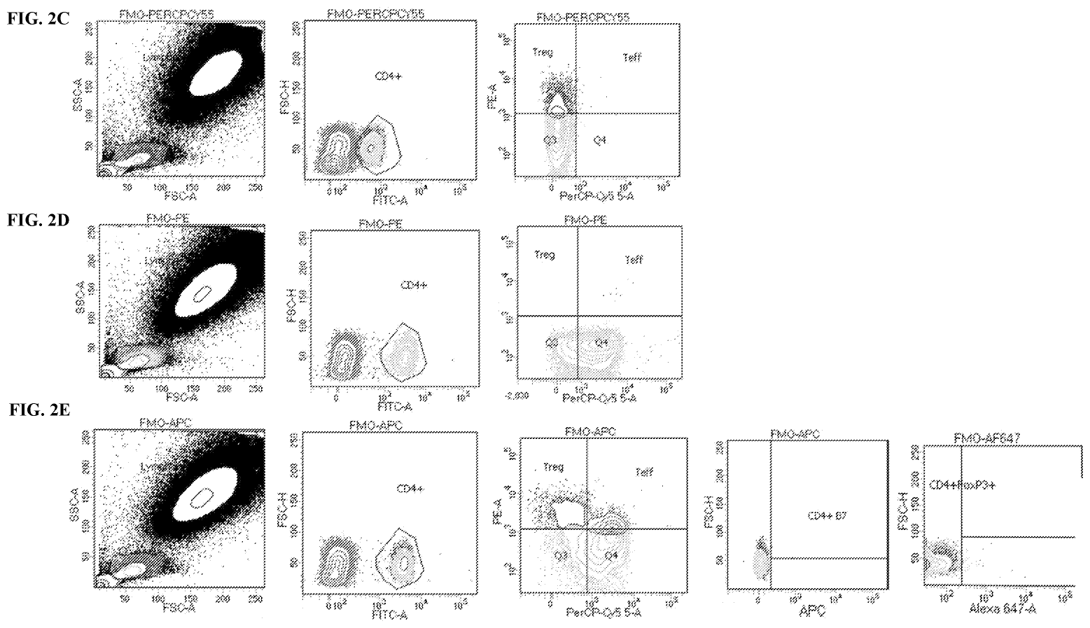

Lastly, peripheral blood cells were stained with fluorescently-conjugated monoclonal antibodies against CD4 (FITC or AF700; RRID: AB_395751 and AB_396943), CD127 (PerCP-Cy5.5; RRID: AB_1645548), CD25 (PE; RRID: AB_400203), FOXP3 (AF647; RRID: AB_1645411), Helios (AF-488; RRID: AB_10661895), CD152/CTLA-4 and/or iCTLA-4 (APC; RRID: AB_398615), CD95/FAS/Apol (APC; RRID: AB_398659), CD39 (APC; RRID: AB_1645459), CD31 (AF647; RRID: AB_397020), CD27 (APC; RRID: AB_1645457), CD45RA (AF700; RRID: AB_1727496), CD45RO (APC; RRID: AB_398673), CCR7 (PE-Cy7; AB_396765), Integrin B7 (APC; RRID: AB_398490) (all from BD Biosciences, San Jose, CA), and CD49d (PE-Cy7; RRID: AB_10643278) (BioLegend Inc., San Diego, CA), with isotype-matched antibodies serving as negative controls. For intracellular markers, cells were permeabilized using the Intracellular Fixation & Permeabilization Buffer Set (eBioscience; catalog #00-5523-00). Extracellular and intracellular labels were examined with an LSR II flow cytometer (BD Biosciences) and analyzed using BD FACSDiva software (RRID: SCR_001456). A representative gating strategy for T cell subset determination is depicted in FIGS. 1A-C and FIGS. 2A-E. CD4+ T cell phenotypes and lymphocyte profiles were compared with those of the previous Phase 1a study (Gendelman H E. et al., NPJ Parkinsons Dis 2017; 3: 10).

TABLE 4
Demographics^{a}

PD Subjects

| | N | Mean (SD) |
|---|---|---|
| Age (years) | 5 | 64 (5) |
| Time Since Diagnosis (years) | 5 | 8 (5) |
| UPDRS Part III Score | 5 | 20 (5) |

| | N | Percentage |
|---|---|---|
| Male Sex | 5 | 100 |
| Caucasian Race | 5 | 100 |
| Anti-Parkinsonian Therapy: | | |
| Carbidopa-Levodopa 25-100 mg^{b} | 3 | 60 |
| Carbidopa-Levodopa 50-200 mg | 1 | 20 |
| Carbidopa-Levodopa 23-95 mg | 1 | 20 |

^{a}Demographic data taken from subjects at the time of enrollment
^{b}Subject 2001 began anti-parkinsonian therapy on month 8

### Regulatory T Cell Proliferation Assays

Regulatory T cell function was assessed at each visit. CD4+CD25+CD127 low cells were isolated using EasySep™ Human CD4+CD127low Enrichment Cocktail and Pan-CD25 Positive Selection and Depletion Kits (Stemcell Technologies, catalog #19232 and 17861) following manufacturer's protocols. Isolated CD4+CD127lowCD25+ Treg were ≥89% pure as determined by flow cytometric analysis. Naïve, CD4+CD25− T responder cells (Tresp) were isolated from a single healthy donor and used for all proliferation assessments. Tresp were labeled with carboxyfluorescein succinimidyl ester (CFSE, Invitrogen, catalog #C34554) and 5×10⁴ Tresp were co-cultured with serially-diluted Treg to yield Treg:Tresp ratios of 1, 0.5, 0.25, 0.125, and 0.0625:1 (Schwab A D. et al., Neurobiol Dis 2020; 137: 104760 and Olanow C W. et al., Ann Neurol 2008; 64 Suppl 2: S101-10) Cellular divisions were tracked by assessing CFSE fluorescence intensity over three days (FIG. 1B). Measures of Treg immunosuppression were determined by calculating the area under the curve (AUC) for both baseline and following sargramostim treatment and by determining the number of Treg needed to achieve 50% inhibition (FIG. 1C).

### Genetic Outcomes

For lymphocyte gene assessments, individual lymphocyte populations were isolated by leukapheresis and centrifugal elutriation, mRNA isolated, and cDNA generated then amplified using quantitative real-time PCR containing gene primers found within RT² Profiler Human Innate and Adaptive Immune Response array (Qiagen, catalog #330231). Fold changes were determined using Qiagen's RT² Profiler analysis software version 3.5, and pathway analyses were performed utilizing Ingenuity Pathway Analysis (IPA; RRID: SCR_008653). To determine methylation status of the Treg specific-demethylated region (TSDR), Treg were also harvested after elutriation, DNA was isolated, and bisulfite methylation pyrosequencing was performed on the FOXP3 locus (Guzman-Martinez L. et al., Front Pharmacol 2019; 10: 1008). Briefly, unmethylated cytosine residues on genomic DNA were deaminated to uracil with bisulfite using the EZ DNA Methylation-Direct kit (Zymo Research, Orange, CA, catalog #D5021) leaving methylated cytosine residues unchanged. Human lymphocyte genomic DNA (Roche Diagnostic Corporation, Indianapolis, IN) methylated using M. SssI (CpG) methylase kit (New England Biolabs, Ipswich, MA) served as high methylation (positive) control, while unmethylated DNA served as low methylation (negative) control. PCR reactions were performed with 42 ng of bisulfite-modified DNA in a total volume of 25 μl and amplified for 35 cycles using Roche Diagnostic Corporation (Indianapolis, IN) FastStart Taq DNA Polymerase (1.0 U), MgCl2 solution (3.5 mM), dNTP's (0.2 mM), sense primer (0.24 uM) (SEQ ID NO: 1), antisense primer (SEQ ID NO: 2) (0.18 μM), with denaturation at 95° C. for 30 seconds, and annealing temperature for 45 seconds at temperature indicated in, and extension at 72° C. for 1 minute. PCR products were electrophoresed in 0.8% agarose gel, stained with ethidium bromide, and visualized for appropriate and pure product before proceeding with analyses using a Bio-Rad Laboratories (Hercules, CA) Gel-Doc UV illuminator. Methylation percentage of each CpG was determined using a Qiagen (Valencia, CA) Pyromark Q96 ID pyrosequencer and sequencing primer (SEQ ID NO: 3) according to manufacturer's recommendations.

### Differential Proteomic Analysis

For proteomic analysis, peripheral blood lymphocytes (PBLs) were isolated by leukapheresis and centrifugal elutriation and kept in liquid nitrogen until processing for proteomic analysis. PBL samples were lysed and protein concentration was determined using a Pierce 660 Protein Assay kit with ionic detergent compatibility reagent (Thermo Fisher Scientific, Rockford, IL, USA) following the manufacturer's instructions. Samples were processed as previously described (Machhi J. et al., Mol Neurodegener 2020; 15 (1): 32) using filter-aided sample preparation (FASP, Pall Corporation, Houston, TX, USA) for digestion of 50 μg per sample. Following overnight digestion, samples were cleaned using Oasis MCX column (Waters Corporation, Milford, MA, USA), followed by C18 Zip-Tips (Millipore Corporation, Billerica, MA, USA). Cleaned peptides were quantitated using NanoDrop2000 at A205.

Following resuspension in 0.1% formic acid, 2 μg of sample was used for label-free quantification (LFQ) in the UNMC Mass Spectrometry & Proteomics Core and the Bioinformatics and Systems Biology Core as previously described (Kosloski L M. et al., J Neuroimmunol 2013; 265 (1-2): 1-10). Briefly, 2 μg of each sample was loaded onto trap column Acclaim PepMap 100 75 μm×2 cm C18 LC Columns (Thermo Fisher Scientific) at a flow rate of 4 μl min⁻¹ then separated with a Thermo RSLC Ultimate 3000 (Thermo Fisher Scientific) on a Thermo Easy-Spray PepMap RSLC C18 75 μm×50 cm C-18 2 μm column (Thermo Fisher Scientific) with a step gradient of 4-25% solvent B (0.1% FA in 80% ACN) from 10-100 min and 25-45% solvent B for 100-130 min at 300 nl min⁻¹ and 50° C. with a 155 min total run time. Eluted peptides were analyzed by a Thermo Orbitrap Fusion Lumos Tribrid (Thermo Fisher Scientific) mass spectrometer in a data-dependent acquisition mode. A survey full scan MS (from m/z 350-1800) was acquired in the Orbitrap with a resolution of 120,000. The automatic gain control (AGC) target for precursor ion scan (MS1) was set as 4×10⁵ and ion filling time set at 100 ms. The most intense ions with charge state 2-6 were isolated in 3 s cycles and fragmented using higher energy collisional dissociation fragmentation with 35% normalized collision energy and detected at a mass resolution of 30,000 at 200 m/z. The AGC target for MS/MS was set at 5×10⁴ and ion-filling time set 60 ms dynamic exclusion was set for 30 s with a 10 ppm mass window.

Protein identification was performed by searching MS/MS data against the SwissProt Homo sapiens protein database downloaded on 21 Oct. 2020 using the in house PEAKS X+DB search engine. The search was set up for full tryptic peptides with a maximum of two missed cleavage sites. Acetylation of protein N-terminus and oxidized methionine were included as variable modifications and carbamidomethylation of cysteine was set as fixed modification. The precursor mass tolerance threshold was set 10 ppm for and maximum fragment mass error was 0.02 Da. The significance threshold of the ion score was calculated based on a false discovery rate of ≤1%. Quantitative data analysis was performed using Progenesis QI Proteomics 4.2 (Nonlinear Dynamics).

### Statistical Analysis

Measurement of Treg function and TSDR correlation evaluations were assessed by linear regression analyses as a function of Treg:Tresp ratio. Differences in Treg suppressive function were determined by differences between groups in slope or elevation. Slopes for all lines were determined to be significantly non-zero. Proteomic data were collected using ANOVA and multiple p values adjusted for false discovery rate of ≤1% using the Benjamini-Hochberg (BH) procedure. The adjusted p≤0.01 was considered as significant. Proteins identified by mass spectrometry were quantified to identify differentially expressed proteins between treatment (two- and six-months post-treatment) and baseline condition (pre-treatment) among all subjects. A protein was considered to be differentially expressed if p-value was ≤0.01 and the absolute fold change was ≥2.

Gene enrichment analysis to identify pathways, functions, and networks affected by treatment were performed using Ingenuity Pathway Analysis (IPA). All other statistical analysis was performed using GraphPad Prism 8.0 software (La Jolla, CA) and Statistica v13.3 (Tibco Software, Palo Alto, CA). All values are expressed as mean±SD. When applicable, differences in between-group means were analyzed using one-way ANOVA followed by Dunnett's post hoc test. Significant differences for these studies, including peripheral blood profiles, MDS-UPDRS Part III motor assessments, flow cytometric analysis, TSDR methylation status, and gene expression analysis was selected at p<0.05. For adverse event profiles, CBCs, T lymphocyte percentages, and clinical chemistries, ANOVA, Fisher's Exact, and/or Mann-Whitney U analyses were performed and are indicated on the corresponding tables. All other correlation analyses were determined using Pearson product-moment correlation coefficients, p values were determined for r values greater than 0.25, and multiple p values adjusted for FDR at 5% by the procedure of Benjamini, Krieger, and Yekutieli. Best-fit lines were determined using linear regression.

### Outcomes

The primary study endpoint was drug safety and tolerability assessed by clinical signs and symptoms, complete blood counts with differential, comprehensive blood chemistry profiles, physical examination, and MDS-UPDRS Part III scores. Hematological profiles were performed by the hospital's clinical diagnostics laboratory, and one neurologist performed all clinical examinations including blood pressure, pulse, temperature, skin, lung, heart, and abdomen evaluations as well as MDS-UPDRS assessments in the “ON” state. Adverse events were recorded and scored based on severity of event as mild (1), moderate (2), or severe (3). Events were also scored in relation to drug treatment as unrelated (1), unlikely (2), possible (3), probable (4), or definitely related (5) (Gendelman H E. et al., NPJ Parkinsons Dis 2017; 3: 10). Mild events caused minimal discomfort or concern and did not interfere with daily activities. Moderate events were defined as discomfort, inconvenience, or concerns ameliorated with simple therapeutic measures. Severe adverse events were defined as discomfort or incapacitation that may require prescription drug therapy, other treatments, or interventions. No events required interruption of treatment. A data and safety monitoring board of UNMC physicians and faculty monitored safety outcomes and advised study investigators during the course of study. Secondary outcomes were immune phenotype and function, DNA methylation status, and gene and proteome analyses.

### Results

### Demographics and Baseline Immune Profiles

Collectively, six PD subjects were enrolled and assessed for eligibility. One subject was excluded due to poor venous access, and the remaining five subjects continued on study for baseline evaluations and treatment (Table 4). All remaining subjects (n=5) were Caucasian males, 57-69 years of age with a mean of 64 years and have been diagnosed with PD for 3-15 years with a mean of 8 years. All subjects displayed complete blood counts, blood chemistry profiles, and immune cell ratios within normal reference values as a requirement for study participation (Table 1-3, baseline column). Due to the wide range of disease duration and potential impact of disease severity on baseline immune profile and treatment-related responses, subjects with abnormal baseline values were excluded. All but one subject began sargramostim therapy while on their anti-Parkinson's medications, as indicated in Table 4. These medications were maintained and continued during the course of study. The remaining subject began anti-parkinsonian treatment at month 8 post-sargramostim initiation.

### Safety, Tolerability, and Adverse Event Profiles

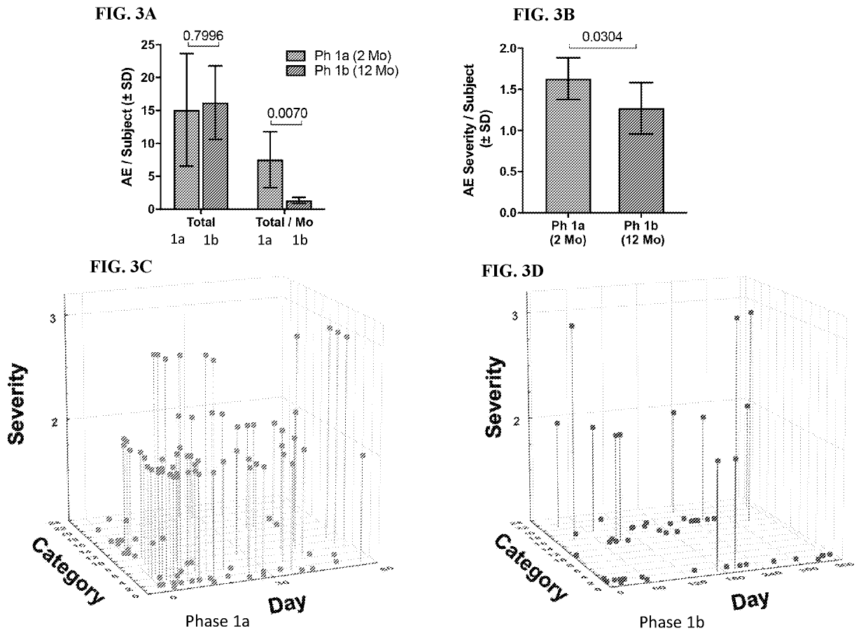

Lowering drug dose and extending treatment to 12 months was safe and generally well-tolerated. However, all subjects reported at least one adverse event over the course of the study, with the majority reporting elevated WBC counts (5/5, 100%), injection site reactions (4/5, 80%), fall with injury (3/5, 60%), and GI tract problems/nausea (3/5, 60%) (Table 5). Less frequently reported adverse events included pain in the upper torso and extremities, chest pain/discomfort, muscle weakness, headache, infection, dyskinesia, and skin and eye problems (1/5, 20% and/or 2/5, 40%). Compared to reported adverse events associated with the Phase 1a study using 6 μg/kg/day for 56 days of treatment (Gendelman H E. et al., NPJ Parkinsons Dis 2017; 3: 10), subjects administered 3 μg/kg/day (5 days on, 2 days off) for 56 days experienced fewer injection site reactions and rashes, less pain in the chest, upper torso, lower torso, and extremities, and less itching, muscle soreness, and weakness. Subjects also displayed significantly less adverse events per subject per month as well as lower severity of adverse events (Table 5 and FIG. 3A-D), with FIG. 3C showing severity of adverse events for phase 1a, and FIG. 3D showing severity of adverse events for phase 1b. Similarly, continued treatment for 12 months, resulted in diminished adverse event profiles. All adverse events in this study were considered mild/moderate with no severe or serious adverse events reported to be associated with treatment and no withdrawals. Two severe adverse events, a viral infection and leg cramping, were reported over the course of 12 months but were deemed to be unrelated to treatment. This is in contrast to the prior study wherein three severe adverse events were reported, and one serious adverse event that led to study withdrawal of one subject (Gendelman H E. et al., NPJ Parkinsons Dis 2017; 3: 10).

As expected, sargramostim increased levels of WBC, lymphocytes, monocytes, eosinophils, and neutrophils (Table 1) and slight increases in CD3+ and CD4+ T cells (Table 2). In addition to lower adverse event profiles, comprehensive metabolic panels for subjects showed no significant increases compared to baseline (Table 4) which validated the safety and tolerability of this low dosage regimen of sargramostim.

TABLE 5
Incidence and severity of adverse events

| | Sargramostim Phase 1a | | Sargramostim Phase 1b | | Sargramostim Phase 1b | |
|---|---|---|---|---|---|---|
| | 6 μg/kg, qd, 2 mos | | 3 μg/kg, 5 d/wk, 2 mos | | 3 μg/kg, 5 d/wk, 12 mos | |
| | (n = 10) | | (n = 5) | | (n = 5) | |
| | Number | Percentage | Number | Percentage | Number | Percentage |
| Adverse Events [symbol] for each subject | | | | | | |
| Any adverse event | 10 | 100 | 5 | 100 | 5 | 100 |
| Any severe adverse events | 3 | 30 | 0 | 0 | 2 | 40 |
| Any serious adverse events | 1 | 10 | 0 | 0 | 0 | 0 |
| Adverse event leading to withdrawal | 4 | 40 | 0 | 0 | 0 | 0 |
| Possible relationship to drug/placebo | 10 | 100 | 8 | 100 | 5 | 100 |
| Definitive relatinship to drug/placebo | 7 | 70 | 3 | 60 | 3 | 60 |
| Category, Subjects reporting | | | | | | |
| 1 Abnomal Laboratory | 10 | 100 | 1 | 20 | 6 | 100 |
| 2 Injection site reaction | 10 | 100 | 4 | 80 | 4 | 80 |
| 3 Chest pain of discomfort | 4 | 40 | 1 | 20 | 1 | 20 |
| 4 Pain, upper torso & extremities | 7 | 70 | 0 | 0 | 1 | 20 |
| 5 Pain, lower torso & extremities | 3 | 30 | 0 | 0 | 0 | 0 |
| 6 Pain, other than exctrimites | 0 | 0 | 0 | 0 | 0 | 0 |
| 7 Rash, other than injection site | 4 | 40 | 0 | 0 | 0 | 0 |
| 8 Itching, other than injection site | 2 | 20 | 0 | 0 | 0 | 0 |
| 9 Edema, other than injection site | 1 | 10 | 0 | 0 | 0 | 0 |
| 10 Shortness of breath, wheezing | 3 | 30 | 0 | 0 | 0 | 0 |
| 11 Headache | 2 | 20 | 1 | 20 | 1 | 20 |
| 12 Fatigue | 2 | 20 | 0 | 0 | 0 | 0 |
| 13 Chills, fever | 2 | 20 | 0 | 0 | 0 | 0 |
| 14 Infection, any | 2 | 20 | 0 | 0 | 2 | 40 |
| 15 GI tract, nausea, vomiting | 3 | 30 | 0 | 0 | 3 | 60 |
| 16 Muscle, soreness, weakness | 3 | 30 | 1 | 20 | 2 | 40 |
| 17 Equilibrium | 1 | 10 | 0 | 0 | 0 | 0 |
| 18 Inury, fall | 2 | 20 | 1 | 20 | 3 | 60 |
| 19 Skin, not infection | 1 | 10 | 0 | 0 | 1 | 20 |
| 20 Cardiovascular, hematological | 2 | 20 | 0 | 0 | 0 | 0 |
| 21 Neurological, psychological, dyskinesia | 2 | 20 | 1 | 20 | 2 | 40 |
| 22 Ophthalmological | 1 | 10 | 0 | 0 | 2 | 40 |
| 23 Sleep anomalies | 1 | 10 | 0 | 0 | 0 | 0 |
| | Median | Mean ± SD | Median | Mean ± 80 | Median | Mean ± SD |
| Total adverse events/subject | 13.5 | 15.1 ± 8.5 | 3.0 | 3.2 ± 1.3 | 17.0 | 16.2 ± 5.5 |
| Total adverse events/subject/ [symbol] | [symbol] . [symbol] | 7.8 ± 4.2 | 0.25 | 0.27 ± 0.11 | 1.4 | 1.4 ± 0.5 |
| Severity of adverse events [symbol] | 1.7 | 1.8 ± 0.3 | 1.0 | 1.2 ± 0.3 | 1.2 | 1.3 ± 0.3 |
| Likelihood of drug-related [symbol] | 3.75 | 3.5 ± 0.7 | 4.2 | 3.8 ± 1.0 | 3.0 | 2.9 ± 1.0 |

[symbol]

Reported adverse events since the initiation of drug [symbol]
More than 2 adverse advents per patient may have been reported [symbol]
Determined by attending physician
p < 0.05 vs. Phare 1a, Mann-Whitney U test [symbol]
indicates data missing or illegible when filed

### MDS-UPDRS Part III Motor Assessment

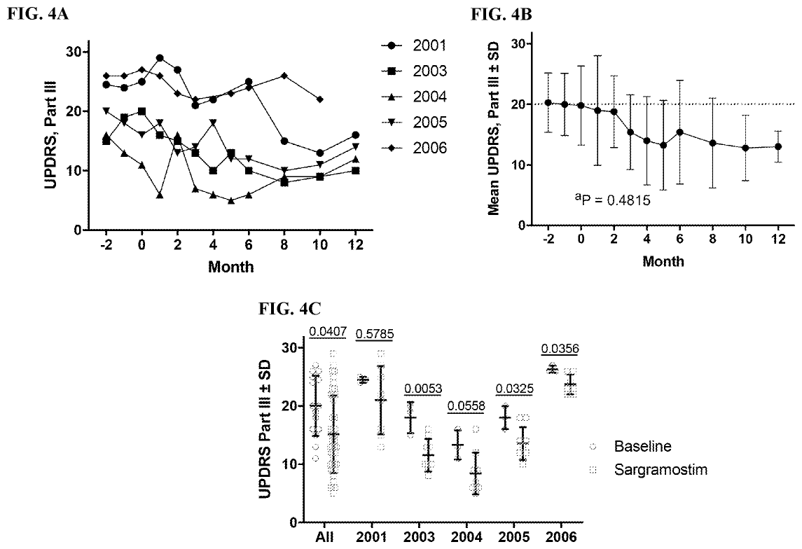

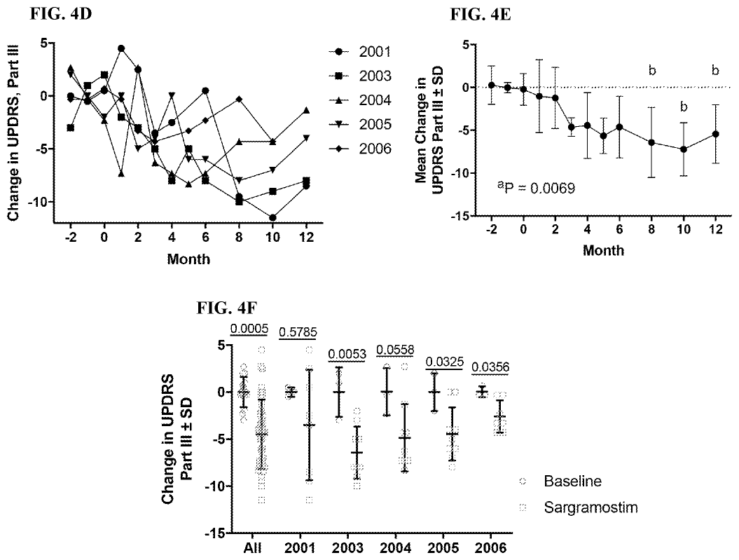

MDS-UPDRS Part III scores were monitored over three months prior to initiating treatment to establish baseline motor function for disease progression monitoring. No worsening of motor function scores was observed for any subject during the course of treatment (FIG. 4A-F). Compared to baseline, sargramostim treatment resulted in an overall decrease in MDS-UPDRS Part III scores for all subjects over time (FIG. 4A). Large variation in raw scores led to non-significant findings (FIG. 4B), however, significance was achieved comparing baseline to cumulative scores (FIG. 4C). Comparison of individual subject baseline and treatment scores demonstrated that 60% (3/5) of subjects displayed decreased MDS-UPDRS Part III scores following sargramostim initiation (FIG. 4C). Normalization of MDS-UPDRS Part III score as a change from baseline showed a profound decrease by three months and was significantly enhanced by eight months (FIGS. 4D and 4E). Similar to total UPDRS readings, 60% (3/5) of subjects experienced significant diminution from baseline at all times during sargramostim treatment (FIG. 4F).

### Immune Modulation and Peripheral Biomarker Evaluations

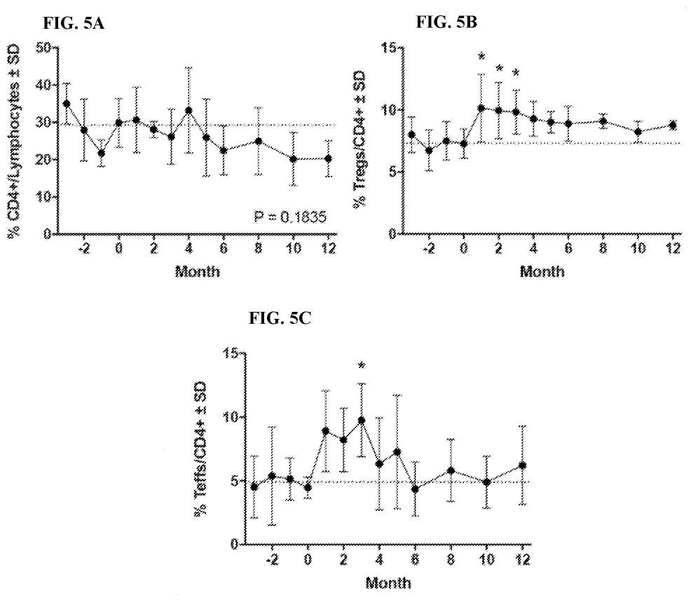

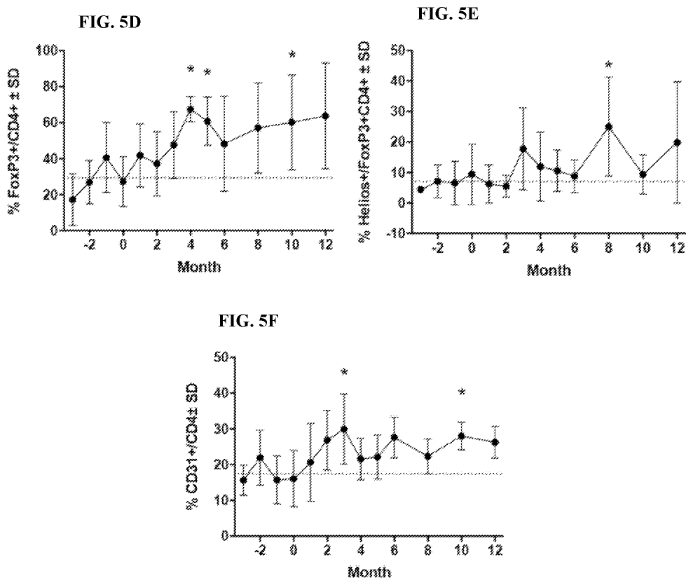

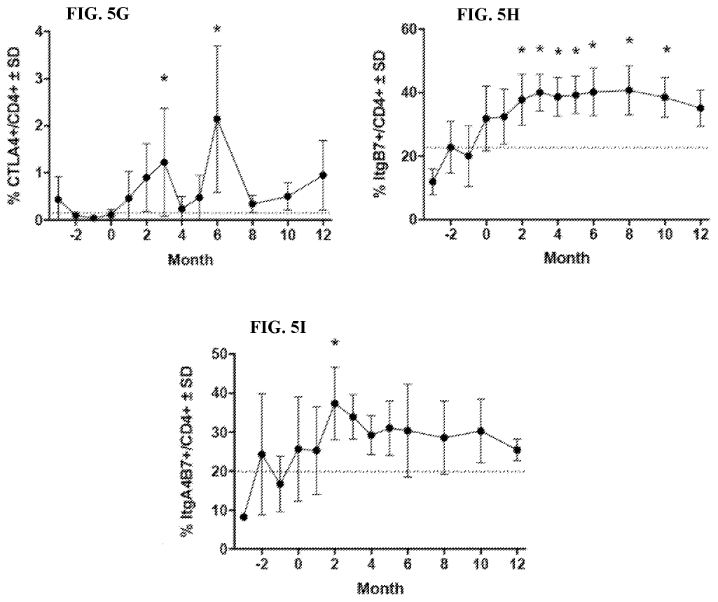

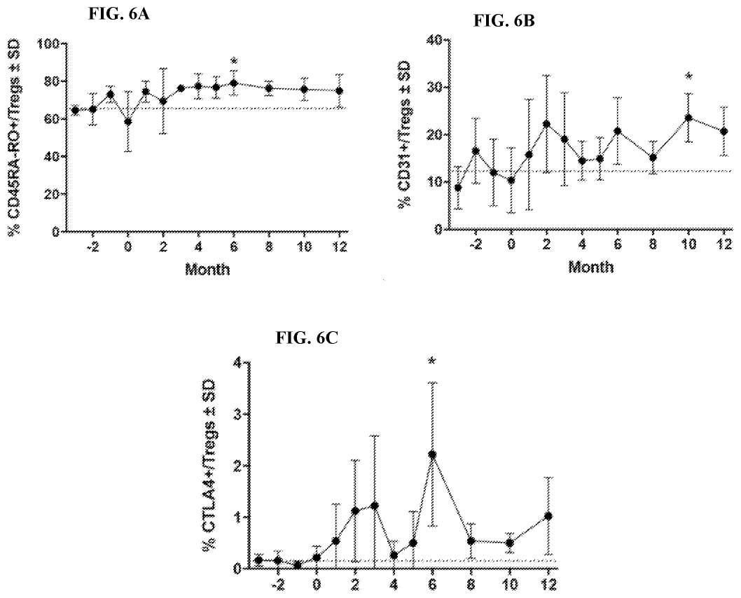

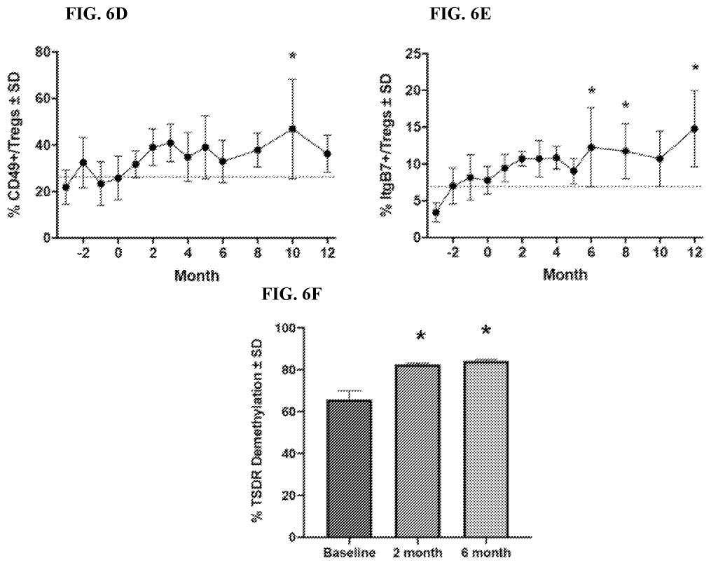

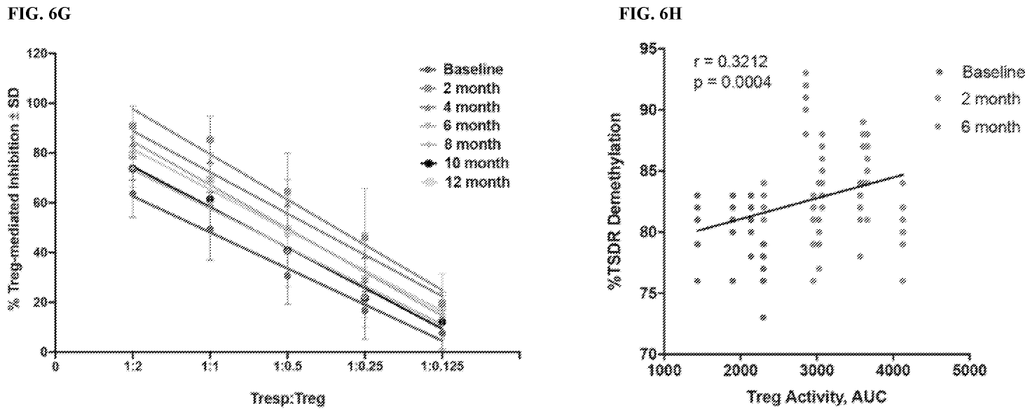

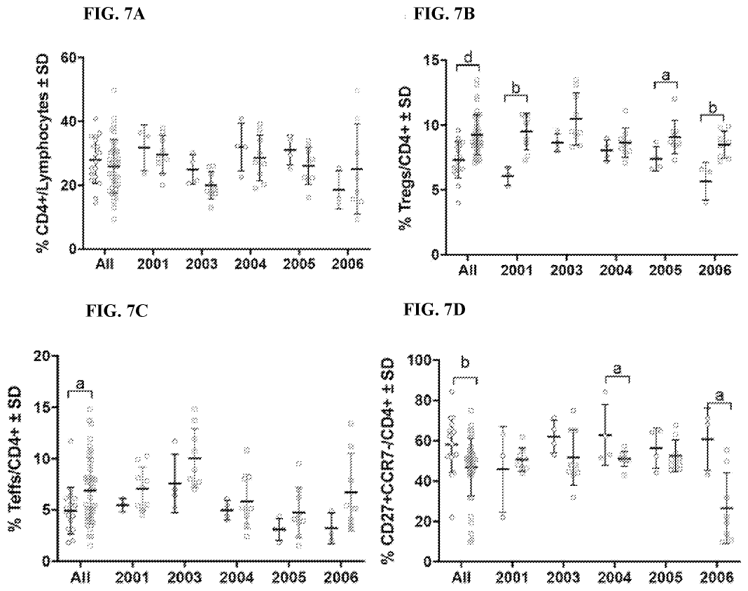

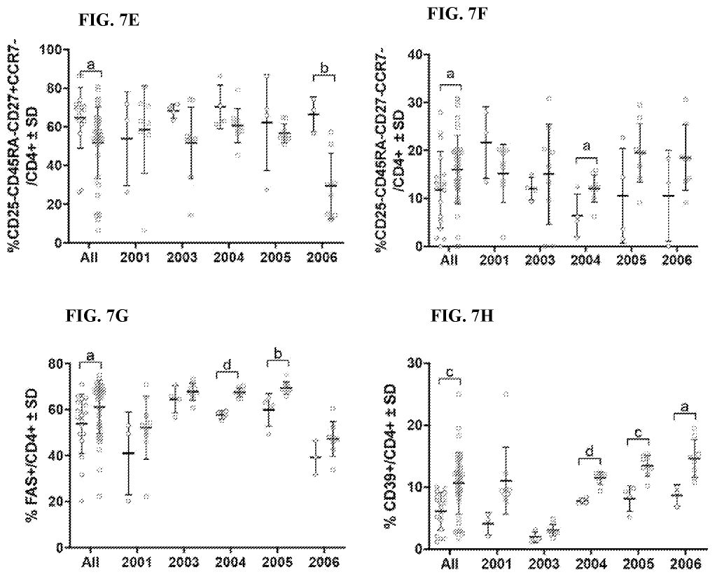

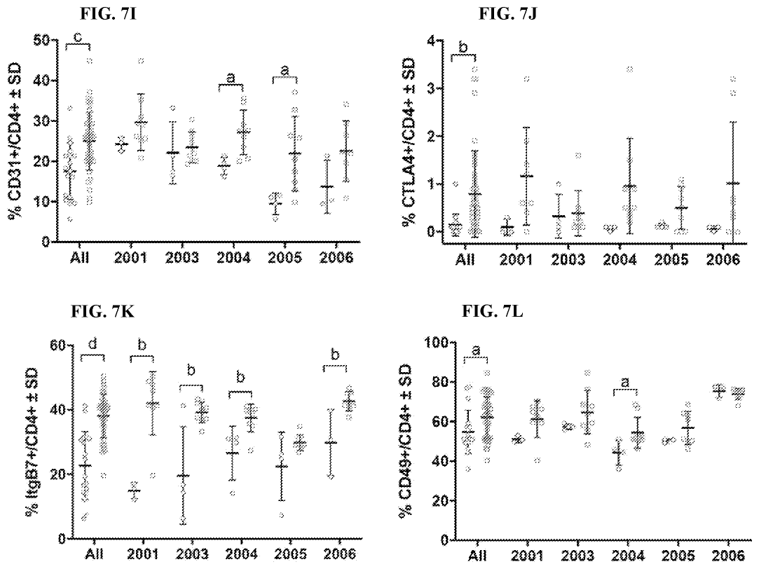

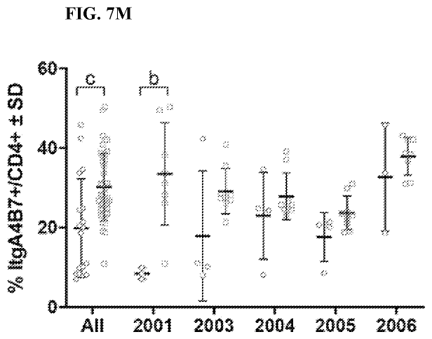

As T cell subsets have been shown to correlate with disease severity, immunomodulatory effects of sargramostim were assessed on phenotypic and functional CD4+ T cell biomarkers (Lindestam Arlehamn C S. et al., Nat Commun 2020; 11 (1): 1875, Gendelman H E. et al., NPJ Parkinsons Dis 2017; 3: 10, Sulzer D. et al., Nature 2017; 546 (7660): 656-61). Flow cytometric analysis revealed no change in total CD4+ lymphocytes and a significant sustained increase in CD4+CD127lowCD25+ Treg and transient increase in CD4+CD127highCD25+ Teff (FIG. 5a-c). Among CD4+ lymphocytes, frequencies of cells expressing FOXP3, Helios, CD31, CTLA, ItgB7, and ItgA4B7 were also increased significantly during treatment (FIG. 5D-I). Subsequent evaluation of Treg subsets also indicated elevated levels of CD45RO, CD31, CD49, CTLA4, and ItgB7 (FIGS. 6A-E). Evaluation of peripheral biomarkers before and after treatment on an individual basis and within the Teff population showed similar patterns (FIGS. 7-10).

From the same Treg isolates, the methylation status of the FOXP3 TSDR was evaluated at baseline following sargramostim treatment. Results indicated that 65% of the TSDR was demethylated at baseline compared to highly methylated DNA controls (FIG. 6F). Sargramostim treatment significantly increased the level of demethylation by 20% at 2 months and maintained demethylation levels at six months which is indicative of stable FOXP3 expression and suppressive Treg phenotype during treatment. Therefore, the effect of sargramostim treatment was assessed on Treg suppressive activities. By 2 months, Treg function was significantly enhanced compared to baseline, and potentiation of Treg function was maintained over the 12-month study (FIG. 4G). Moreover, levels for Treg-mediated activity and FOXP3 TSDR demethylation were confirmed to be positively correlated (r=0.3212, p=0.0004), thus verifying the effect of sargramostim on FOXP3 TSDR-directed Treg immunosuppressive capacity (FIG. 6H (4h)).

### Flow Cytometric Analysis of CD4+ T Cell Subsets

Comparison of mean CD4+ T cell frequencies combined before or during treatment for all or individual subjects confirmed no significant differences in CD4+ cells (FIG. 2A). However, among the CD4+ T cell subsets in the combined cohort (All), mean frequencies of Treg and Teff were increased with significant increases in 3/5 subjects, CD4+CD27+CCR7− and CD24+CD25−CD45RA−CD27+CCR7− subsets were decreased, and subset phenotypes included those co-expressing CD4+CD25−CD45RA−CD27−CCR7−, CD4+FAS+, CD4+CD39+, CD4+CD31+, CD4+CTLA+, CD4+ItgB7+, CD4+CD49+, and CD4+ItgA4B7+(FIG. 2B-M). A minor and transient increase in CD4+CD25+CD127high Teff was observed following sargramostim treatment.

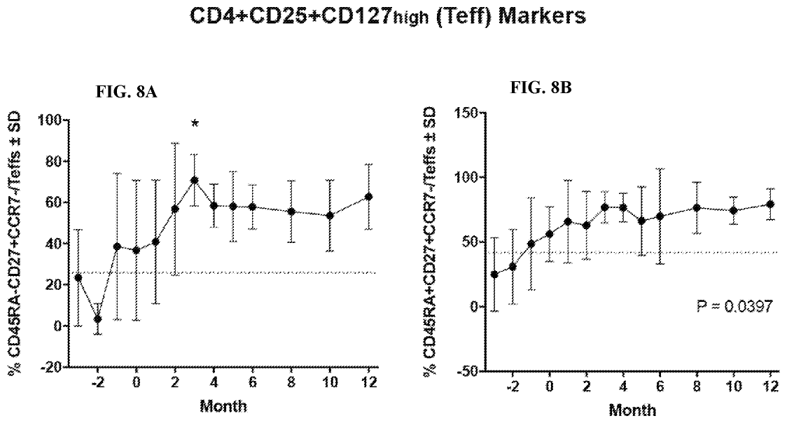

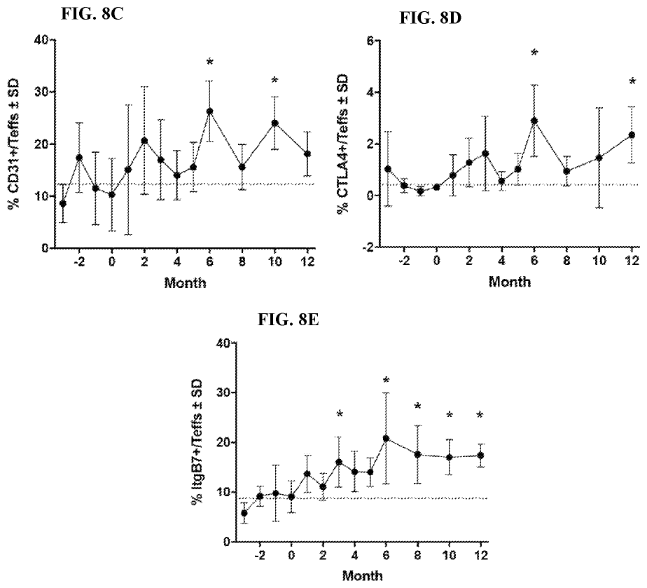

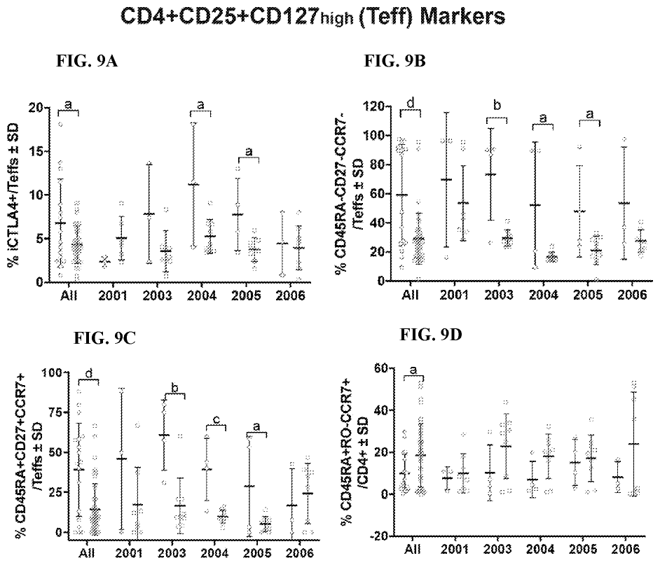

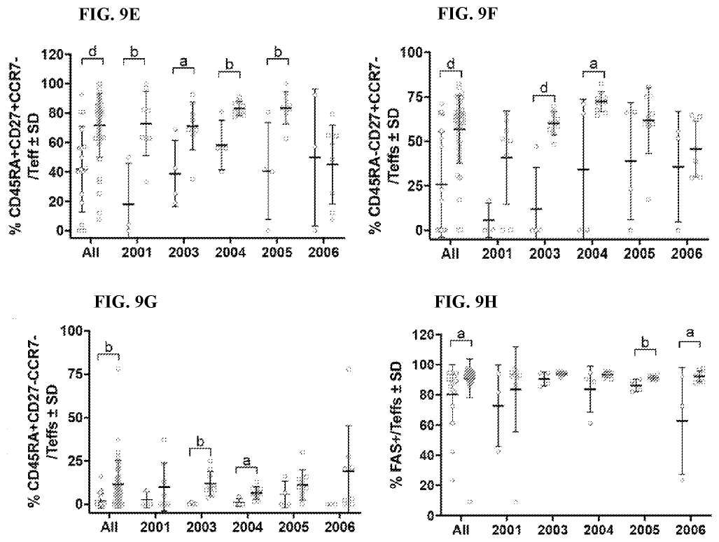

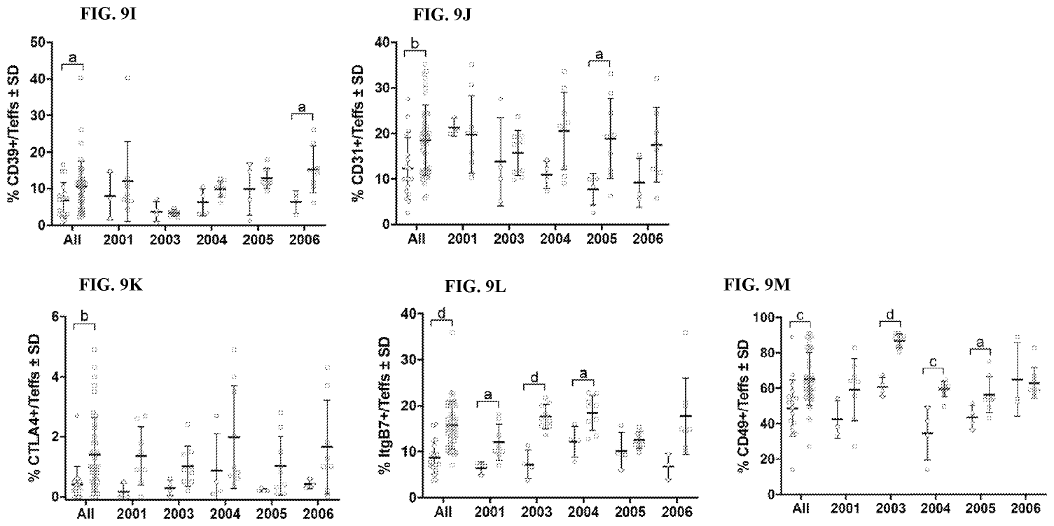

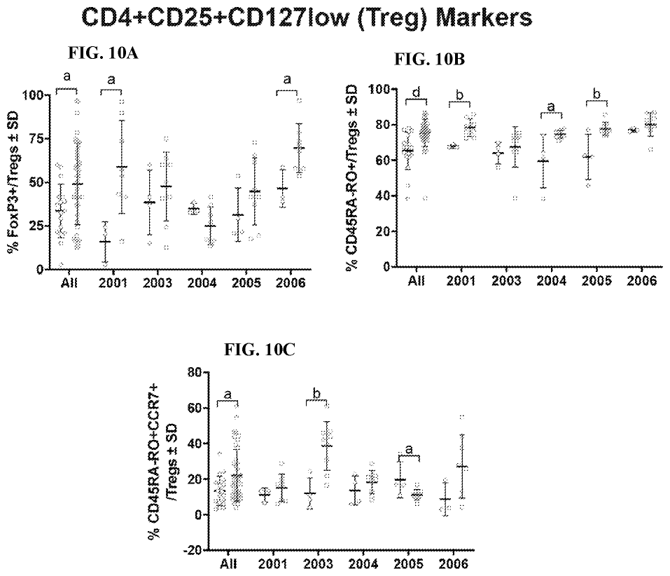

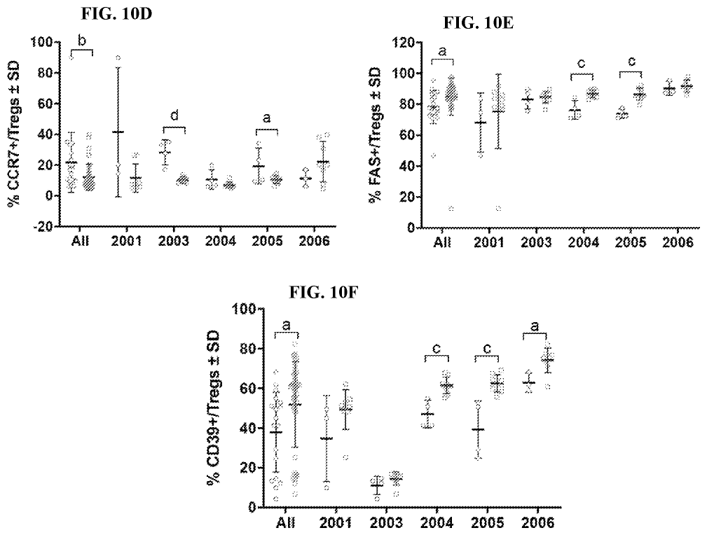

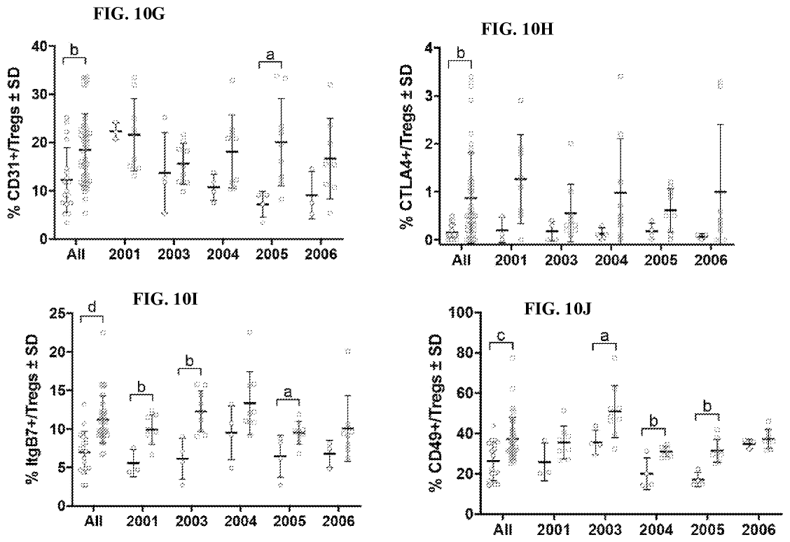

A significant increase in CD45RA−CD27+CCR7− and CD45RA+CD27+CCR7−, CD31+, CTLA+, and ItgB7+ Teff was observed over time, similar to the expression observations in Treg populations (FIG. 7A-E). Combining subset frequencies for all cohort time points validated those results showing sargramostim treatment diminished mean levels of iCTLA+, CD45RA−CD27−CCR7−, and CD45RA+CD27+CCR7+ Teffs with increased levels of CD45RA+RO−CCR7+, CD45RA+CD27+CCR7−, CD45RA−CD27+CCR7−, and CD45RA+CD27−CCR7− Teffs as well as those that co-express FAS, CD39, CD31, CTLA, ItgB7, CD49 (FIGS. 8A-M). Sargramostim treatment also increased levels of Tregs that co-express FOXP3, CD45RO, CCR7, FAS, CD39, CD31, CTLA, ItgB7, and CD49 (FIGS. 9A-J). Correlation analyses using the observed markers assessed against Treg function by defining function as Treg number necessary to yield 50% inhibition resulted in inverse correlations for the percentage of CD4+ T cells or Treg that express ItgB7, ItgA4B7, FAS, CD39, CD45RA, or and CD27 and more potent Treg activity (FIGS. 10A-G).

Association of Peripheral T Cell Biomarkers with Treg Cell Function and MDS-UPDRS Part III Scores

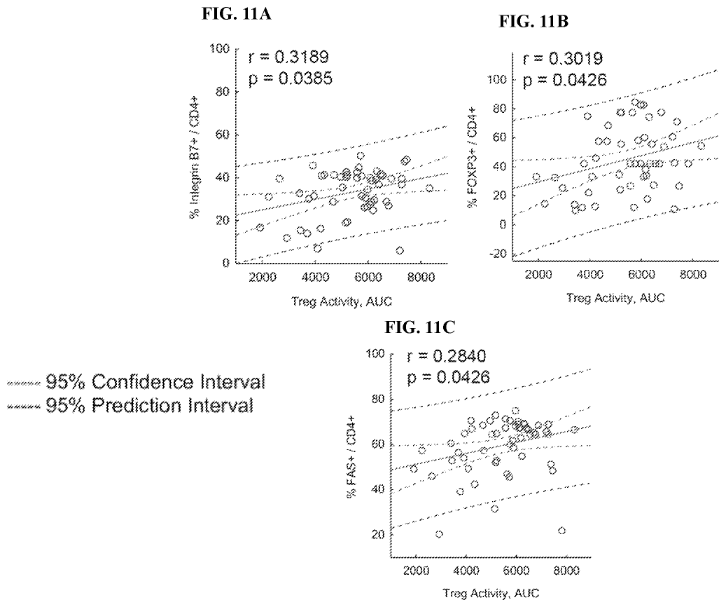

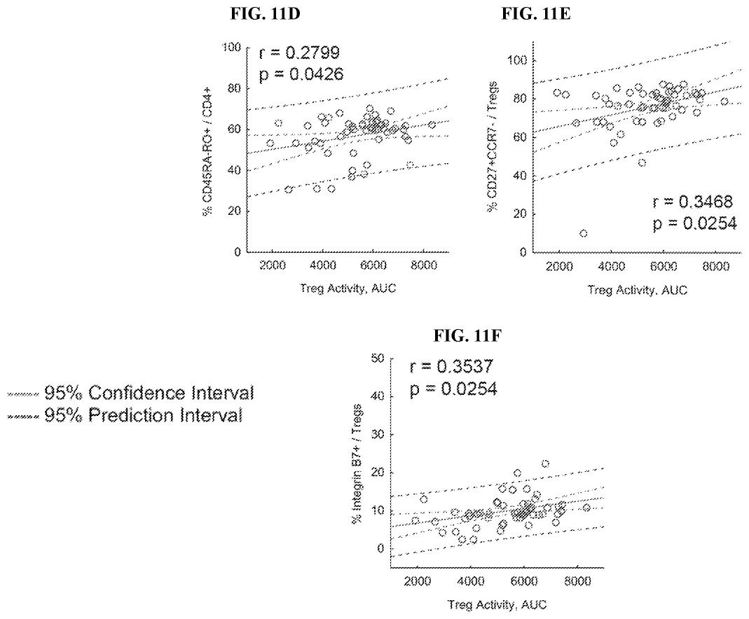

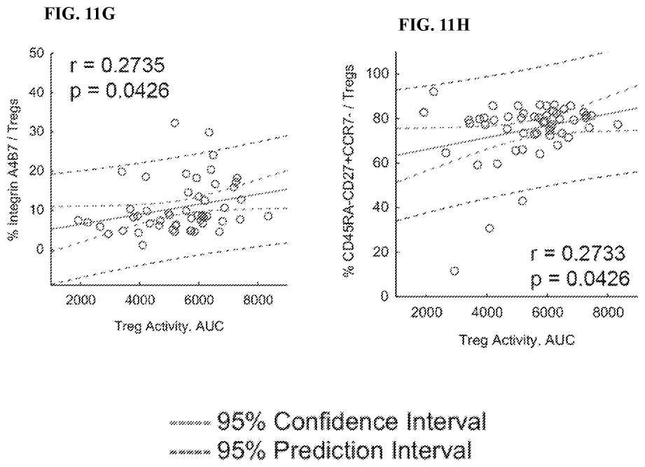

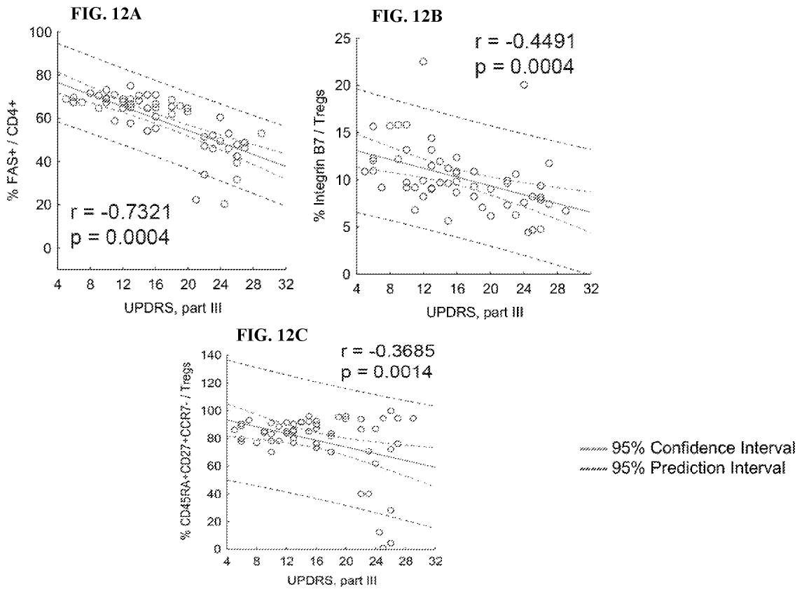

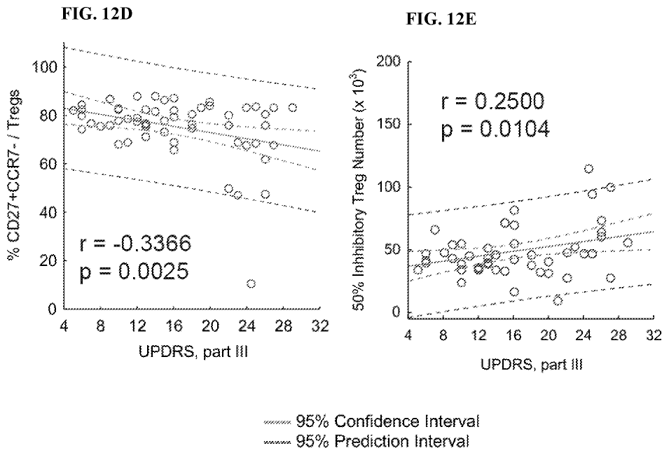

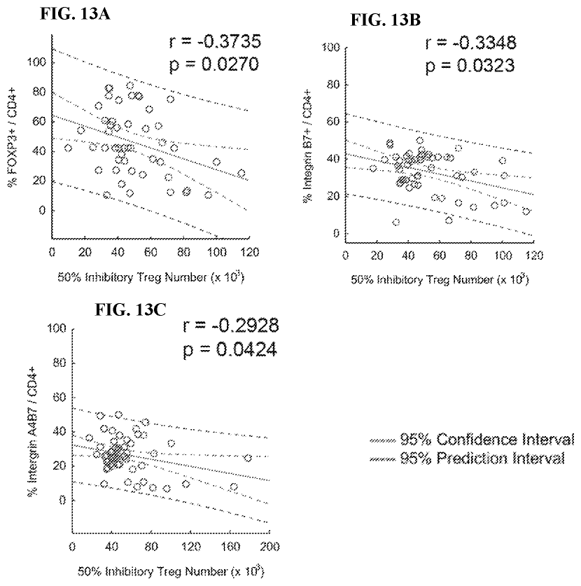

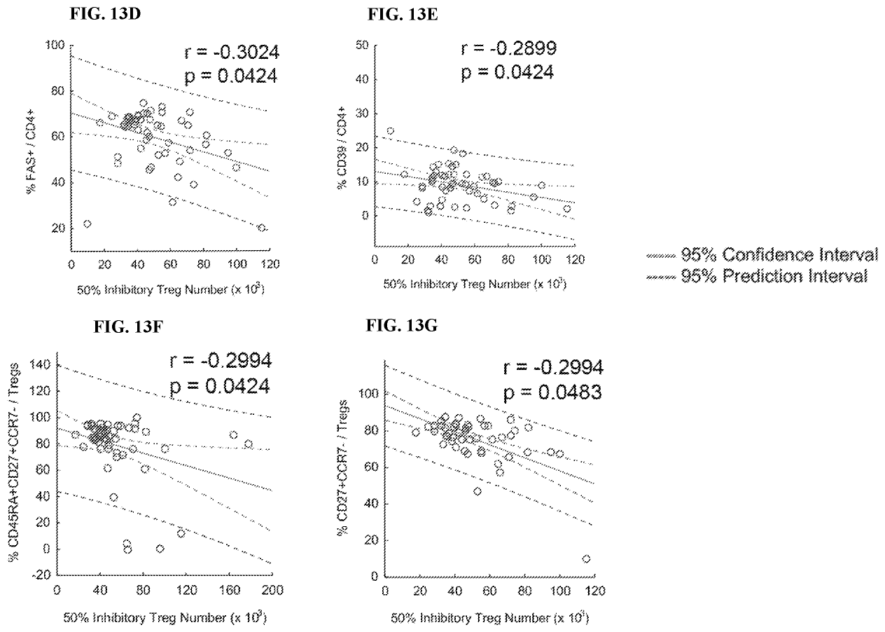

Next, the effect of sargramostim on treatment biomarker expression with Treg-mediated immunosuppressive function was assessed (FIGS. 11A-H). First, Treg activity was positively associated with increased co-expression of ItgB7, FOXP3, FAS, CD27, and CD45RA (FIGS. 11A-H). Second, negative correlations were shown between increasing MDS-UPDRS Part III scores and diminished frequencies of FAS+CD4+ T cells and Treg subsets that co-express ItgB7, CD45RA, and CD27 with the lack of CCR7 (FIGS. 12A-D), suggesting that motor function is improved with increased Treg subset levels and greater suppressive activity. Indeed, this was confirmed by the positive correlation of increased Treg function as measured by lower number of Tregs to yield 50% suppression and diminished MDS-UPDRS Part III scores (FIG. 12E; FIGS. 13A-G).

### Gene Profiling of Lymphocyte Populations

Lastly, a putative mechanism was sought by which sargramostim affects immune regulation of disease severity through transcriptomic profiling of peripheral blood lymphocytes (PBLs) before and during treatment. Heat maps of transcriptomic changes from baseline were detected in total peripheral blood lymphocytes and showed mixed phenotypes among individual subjects at two and six months of sargramostim treatment (FIG. 13A). Of the 84 genes assessed, significant gene expression levels over baseline were observed in each subject, although to varying degrees (FIG. 13B). However, among all subjects at two and six months of sargramostim treatment, greater than two-fold increases were shown in MBL2, IFNA1, IL10, and APCS.

### Proteomic Profile or Peripheral Blood Lymphocytes.

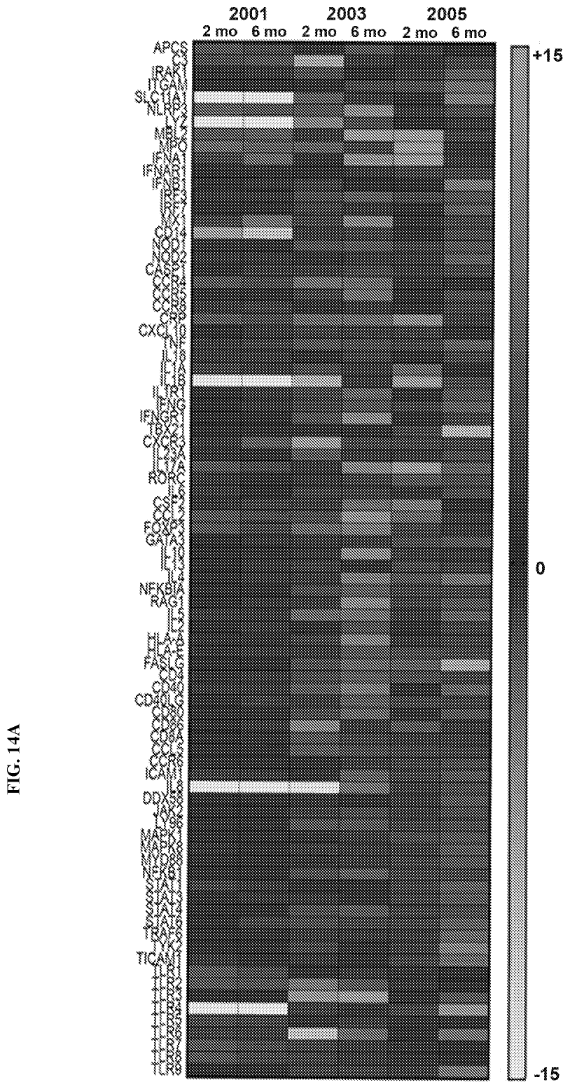

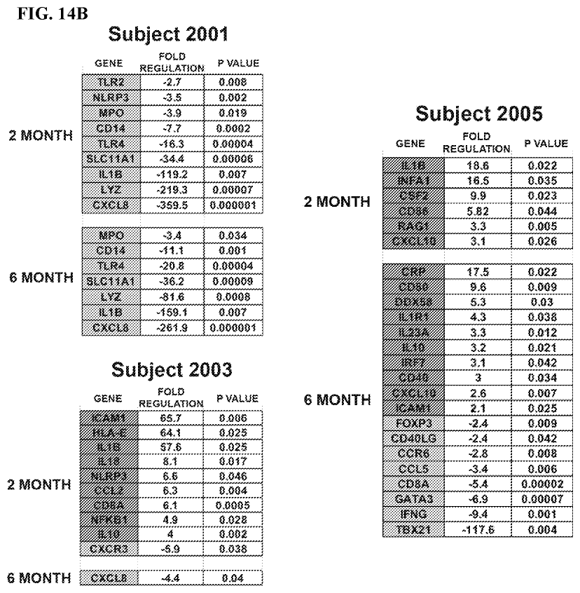

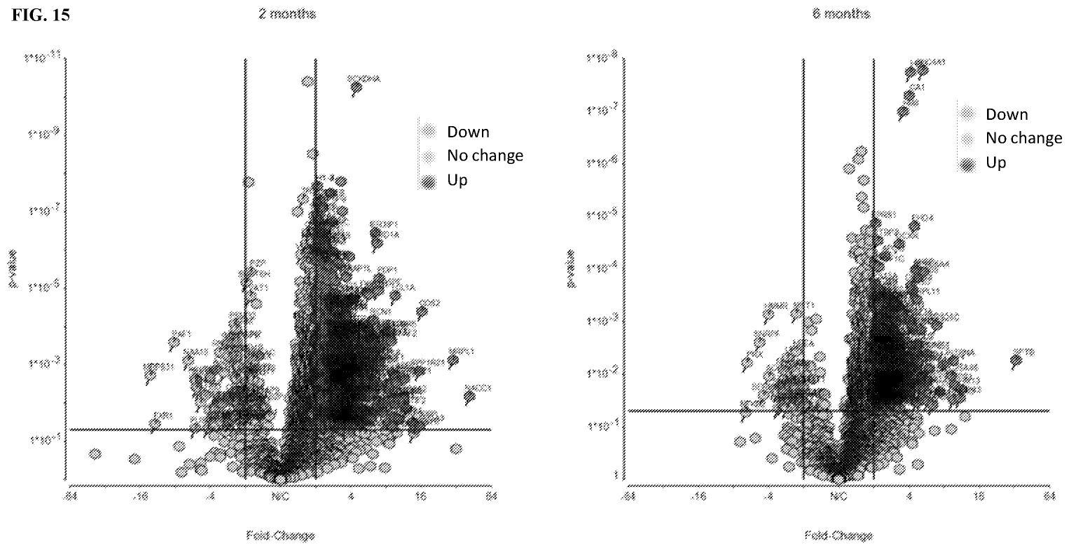

Before sargramostim initiation and at 2 and 6 months of sargramostim treatment, PBL isolated by leukapheresis were subjected to transcriptomic (FIGS. 14A-B) and proteomic analyses. Expression of over 2,500 proteins were identified and quantified for analysis. Proteins whose expression was significantly down-regulated (light shading) or upregulated (dark shading) post-treatment as compared with pre-treatment are shown in FIG. 14B. Among these proteins, 785 and 152 proteins were significantly differentially expressed at 2 and 6 months of sargramostim treatment, respectively. Volcano plots highlighting the proteins whose expression was significantly down-regulated (light shading) or upregulated (dark shading) post-treatment as compared with pre-treatment are shown in FIG. 15. Ingenuity Pathway Analysis (IPA) comparison analysis of canonical pathways altered at both 2 and 6 months of treatment showed activation of 25 pathways (Table 6) identified by selected association with cellular immune response signaling, neuroinflammation signaling, and PD signaling pathways. Interestingly, 15 out of 25 pathways showed a downregulation of calcineurin and/or nuclear factor kappa B (NF-κB) expression at 2 months but not 6 months after treatment (Table 6).

TABLE 6
IPA comparison analysis of canonical pathways

| | Activation z-score | Activation z-score |
|---|---|---|
| | during sargramostim | during sargramostim |
| | treatment | treatment |
| Canonical Pathways | 2 Months | 6 Months |
| IL-8 Signaling | 4.9 | 3.7 |
| fMLP Signaling in Neutrophils (a + b) | 4.3 | 3.7 |
| Role of NFAT in Regulation of the Immune Response (a + b) | 4.1 | 3.9 |
| Fcγ Receptor-mediated Phagocytosis in Macrophages and Monocytes | 4.3 | 3.5 |
| CXCR4 Signaling | 4.0 | 3.6 |
| PKCθ Signaling in T Lymphocytes (b) | 3.9 | 3.6 |
| Leukocyte Extravasation Signaling | 4.1 | 3.2 |
| CD28 Signaling in T Helper Cells (a + b) | 3.7 | 3.6 |
| iCOS-iCOSL Signaling in T Helper Cells (a + b) | 3.7 | 3.0 |
| Calcium-induced T Lymphocyte Apoptosis (a) | 3.8 | 2.4 |
| Natural Killer Cell Signaling (b) | 2.8 | 3.4 |
| PI3K Signaling in B Lymphocytes (a + b) | 3.1 | 2.8 |
| CCR3 Signaling in Eosinophils | 3.0 | 2.6 |
| IL-3 Signaling (a) | 2.7 | 2.4 |
| GM-CSF Signaling (a) | 2.5 | 2.4 |
| Macropinocytosis Signaling | 2.3 | 2.4 |
| IL-7 Signaling Pathway | 1.7 | 3.0 |
| Interferon Signaling | 2.3 | 2.2 |
| Nur77 Signaling in T Lymphocytes (a) | 3.2 | 1.0 |
| IL-9 Signaling (b) | 2.1 | 2.0 |
| Antiproliferative Role of TOB in T Cell Signaling | −1.4 | −2.2 |
| CCR5 Signaling in Macrophages | 3.5 | N/A |
| Role of PKR in Interferon Induction and Antiviral Response (b) | 2.0 | 1.3 |
| Production of Nitric Oxide and Reactive Oxygen Species in Macrophages (b) | 2.3 | 0.8 |
| iNOS Signaling (b) | 2.1 | NA |

(a) Downregulation of calcineurin expression
(b) Downregulation of NF-κB expression
(c) Downregulation of calcineurin and NF-κB expression

### Discussion

Treatment with sargramostim at 3 μg/kg/day (5 days on, 2 days off) for 12 months was generally well-tolerated. Commonly reported adverse events were associated with known side effects including increased injection site reactions, elevated WBC counts, and bone pain (Baldo B A. Drug Saf 2014; 37 (11): 921-43). No serious or severe adverse events were reported as being linked to drug administration and no subjects were withdrawn from treatment, further supporting the drug's safety and tolerability for this indication. Chronic treatment also did not result in disease worsening in any subject as determined by MDS-UPDRS Part III scores before and during treatment. With dopamine replacement therapies, UPDRS Part III scores increase in a linear fashion by an average of 2.4 points per year over the course of five years (Holden S K. et al., Mov Disord Clin Pract 2018; 5 (1): 47-53).

In the current study, which includes adjunctive sargramostim, four subjects collectively decreased MDS-UPDRS part III scores, and one subject maintained baseline scores. While UPDRS part III motor assessment to evaluate clinical efficacy can be subjective and has inherent limitations due to variation, it remains the gold standard in PD for evaluation of motor dysfunction during clinical assessments (Evers L J W et al., Mov Disord 2019; 34 (10): 1480-7). However, clinical efficacy utilizing motor improvement evaluations in PD are difficult to assess due to the known placebo effect in this disease population (Quattrone A. et al., Mov Disord 2018; 33 (8): 1213-27). This effect in placebo-treated subjects was previously observed for six weeks post-treatment initiation in the initial Phase 1 clinical trial (Gendelman H E. et al., NPJ Parkinsons Dis 2017; 3: 10). Although, by seven weeks, placebo controls returned to baseline UPDRS Part III scores, while scores of sargramostim-treated subjects continued to decline and remained below baseline values and those of placebo-treated subjects until discontinuation. Therefore, the effect observed over the course of a year is noteworthy, but cannot be verified until a larger-scale, placebo-controlled study powered for clinical efficacy is performed.

Low-dose sargramostim positively altered immune function, shifted T cell phenotypes, and enhanced treatment-induced biomarker levels that were associated with lowered MDS-UPDRS Part III scores. Treg and Teff frequencies were increased within one month after sargramostim initiation with parallel increases in CD4+ T cell biomarkers. These biomarkers were associated with cell homing, such as Itgβ7 and Itgα4β7, as well as signaling and anti-inflammatory mechanisms for Treg-mediated immunosuppression that include FAS, CD49, CD31, FOXP3, CTLA4, and CD39. Increased levels of Itgβ7 and Itgα4β7 are likely associated with increased migratory capacity of T cells homing to sites of inflammation and MAdCAM-1 within the gut (DeNucci C C. et al., J Immunol 2010; 184 (5): 2458-67, Stassen M. et al., Eur J Immunol 2004; 34 (5): 1303-11), but can also bind to vascular CAM-1 (VCAM-1) under inflammatory conditions (Swerlick R A. et al., J Immunol 1992; 149 (2): 698-705), playing a role in progression of chronic forms of neurodegenerative disease (Kanwar J R. et al., J Neuroimmunol 2000; 103 (2): 146-52). Moreover, Tregs expressing Itgα4β7 display higher suppressive function than those lacking expression by enhancing IL-10 secretion and inducing other regulatory-like T cell phenotypes (Stassen M. et al., Eur J Immunol 2004; 34 (5): 1303-11). Increases in IL-10 gene expression by PBLs was observed from sargramostim-treated subjects suggesting an anti-inflammatory role for sargramostim. Blockade of Itgβ7 is also linked to increased inflammation due to impaired homing and migration of Tregs, further strengthening the notion that upregulation of integrin biomarkers is beneficial for repair or replenishment of dysfunctional Treg populations as found in PD (see, e.g., Sun H, et al., Cell Mol Gastroenterol Hepatol 2020; 9(3): 369-85; Klann J E et al., J Immunol 2018; 200(12): 4012-23; Lehmann J et al., Proc Natl Acad Sci USA 2002; 99(20): 13031-6).

Expression of CD49 may reflect a maturation biomarker for T cells as development of functionally mature Treg have been linked to expression of CD4927. Absence of CD31 or platelet endothelial cell adhesion molecule (PECAM-1) expression is associated with Treg dysfunction (Huang L. et al., Clin Exp Immunol 2017; 187 (3): 441-54). Low frequency of CD31+ Treg has been connected to decreased FOXP3 expression in coronary heart disease and Treg dysfunction in multiple sclerosis (Huang L. et al., Clin Exp Immunol 2017; 187 (3): 441-54, Haas J. et al., J Neuroimmunol 2009; 216 (1-2): 113-7). Similarly, increased FOXP3 expression is indicative of enhanced immunosuppressive function, as its presence is required to maintain a stable suppressive Treg phenotype (Lu L. et al., Nat Rev Immunol 2017; 17 (11): 703-17).

Along with FOXP3 expression, Tregs utilize CTLA4 and CD39 to maintain suppressive capability (Shevyrev D. et al., Front Immunol 2019; 10: 3100). Both are considered to be aligned with potent mechanisms of immunosuppression. CTLA4 expression controls antigen presentation by inhibiting co-stimulation via CD80/CD86 blockade (Walker L S. J Autoimmun 2013; 45: 49-57, Wing J B. et al., Immunity 2014; 41 (6): 1013-25). CD39 is an ectonucleotidase that converts ATP into AMP that can metabolically starve surrounding cells, thus stunting cellular division (Borsellino G. et al., Blood 2007; 110 (4): 1225-32). CD73, another Treg-associated ectonucleotidase, converts AMP to adenosine that can interact with purinergic receptors on Teff to elevate intracellular cAMP and suppress proliferation (Borsellino G. et al., Blood 2007; 110 (4): 1225-32, Deaglio S. et al., J Exp Med 2007; 204 (6): 1257-65). CD39+ Treg also maintains strong suppression and functional stability in the presence of inflammatory stimuli such as IL-1β and IL-6, which are both upregulated in PD (Chao Y. et al., Biomed Res Int 2014; 2014: 308654, Gu J. et al., Cell Mol Immunol 2017; 14 (6): 521-8, Stojakovic A. et al., Mol Neurobiol 2017; 54 (6): 4486-95). Specifically, within Treg and Teff subsets, increased CCR7, CD27, and CD45RO expression following sargramostim treatment was observed. Increased CCR7 and CD27 expression is associated with migration and enhanced function of effector memory-like Treg and with maintenance of Treg circulation within the periphery (Borst J. et al., Curr Opin Immunol 2005; 17 (3): 275-81, Menning A. et al., Eur J Immunol 2007; 37 (6): 1575-83, Cowan J E. et al., Cell Rep 2016; 14 (5): 1041-8).

Lastly, a significant elevation in CD45RA−CD45RO+ Tregs was observed which is indicative of memory and past activation, as well as enhanced suppressive function, suggesting that sargramostim is inducing Treg with high proliferative capacity, potentially leading to the observed increased cell frequency (Booth N J. et al., J Immunol 2010; 184 (8): 4317-26). With increased Treg subsets, CD4+CD25+CD127high Teff were also increased in the first months post-treatment; however, increased Teff prevalence was not observed after four months. Although, Teffs expressing CD27, CTLA4, ItgB7, and CD31 were significantly elevated with treatment. CD27 promotes cell survival, and increased expression of CTLA4 and ItgB7 is likely associated with cell activation. Also, the presence of CD31+ Teff has been positively associated with lower UPDRS Part III scores (Saunders J A. et al., J Neuroimmune Pharmacol 2012; 7 (4): 927-38). Therefore, the potential effect of concomitantly inducing these effector populations alongside Treg does not appear to be deleterious nor has a negative impact on Treg function and the overall immunosuppressive phenotype afforded by sargramostim treatment. Additionally, GM-CSF is known to expand other immunosuppressive cell populations such as myeloid-derived suppressor cells (MDSCs), regulatory B cells, and/or tolerogenic dendritic cells (Park M Y. et al., Front Immunol 2019; 10: 183, Hamilton J A. et al., Trends Immunol 2013; 34 (2): 81-9, Pulendran B. et al., J Immunol 2000; 165 (1): 566-72, Bhattacharya P. et al., Cytokine 2015; 75 (2): 261-71, Sheng J R. et al., J Immunol 2014; 193 (6): 2669-77, Schutt C R. et al., Mol Neurodegener 2018; 13 (1): 26). These populations were not evaluated in the current study, but it should be noted that any clinical effects observed may also be mediated, in part, by the presence and induction of this immunoregulatory population as well. Pre-clinical evaluations utilizing the MPTP mouse model of PD indicate the ability of GM-CSF to induce tolerogenic bone marrow-derived dendritic cells that likely contribute to the increased presence of Treg following treatment (Schutt C R. et al., Mol Neurodegener 2018; 13 (1): 26).

Furthermore, a clinical study investigating immunoregulatory cell populations within 32 PD subjects indicated decreased levels of suppressor and activated Tregs, IL-10 producing CD8+ Tregs, and tolerogenic dendritic cells, further supporting the notion that PD subjects have an impaired ability to suppress proinflammatory responses (Alvarez-Luquin D D. et al., J Neuroinflammation 2019; 16 (1): 212). Therefore, the potential effect of GM-CSF on other regulatory phenotypes would open an exciting avenue to explore for future investigation.

Concordant with the observed Treg and Teff biomarker increases, sargramostim treatment enhanced Treg-mediated immunosuppressive function that was maintained over the course of the study. Previously, Tregs isolated from PD subjects showed impaired ability to suppress Teff proliferation that correlated with increased disease severity (Saunders J A. et al., J Neuroimmune Pharmacol 2012; 7 (4): 927-38). Treg deficiency has been associated with increased disease progression in Alzheimer's disease, ALS, stroke, traumatic brain injury, and multiple sclerosis (Machhi J. et al., Mol Neurodegener 2020; 15 (1): 32). These reports and previous works suggest that controlling neuroinflammation via Treg induction or enhancement may be a promising therapeutic avenue for the clinic (Gendelman H E. et al., NPJ Parkinsons Dis 2017; 3: 10, Beers D R. et al., JCI Insight 2017; 2 (5): e89530, Faridar A. et al., Brain Commun 2020; 2 (2): fcaa112). Here, sargramostim treatment induced Tregs and restored Treg function via increased demethylation of FOXP3 TSDR and enhanced expression of biomarkers necessary to maintain a suppressive phenotype. This was indicated by the correlation between Treg activity, methylation status, and levels of peripheral T cells expressing Treg biomarkers. Demethylation of the TSDR is responsible for maintaining stable FOXP3 expression and Treg function, while hypermethylation of the TSDR is associated with Treg dysfunction in other diseases (Schreiber L. et al., PLOS One 2014; 9 (2): e88318, Anderson M R. et al., J Neuroimmune Pharmacol 2014; 9 (4): 522-32, Shimazu Y. et al., Cancer Immunol Res 2016; 4 (2): 136-45). This study shows that sargramostim treatment leads to hypomethylation and increased FOXP3 levels, positively impacting and restoring Treg function. However, although significantly elevated throughout the course of treatment, Treg function peaked 2 months post-drug initiation, slowly decreasing in effectiveness over time. This decrease may be due to decreased capacity for induction of Treg over the extended treatment, exhaustion of bone-marrow derived cell production, and/or the presence of neutralizing anti-drug antibodies (Gendelman H E. et al., NPJ Parkinsons Dis 2017; 3: 10). Previously, low levels of anti-sargramostim antibodies within the serum were detected one month after treatment.

Overall, transcriptomic and proteomic analyses of PBLs during sargramostim treatment revealed an activated phenotype (FIG. 15, FIG. 13, and Table 5). Gene dysregulations involved both pro- and anti-inflammatory mediators, suggesting that low-dose sargramostim treatment results in immune activation within the PBL population similar to observations within the CD4+CD25− Teff and Treg subsets isolated in previous high-dose treatment (Gendelman H E. et al., NPJ Parkinsons Dis 2017; 3: 10). However, the lower dosage sargramostim regimen reported here also resulted in increased IL-10 gene expression at both 2 and 6 months after treatment initiation, supporting the immunosuppressive biomarker expression observed in flow cytometric analysis and Treg function.

Previous findings also support the notion that Treg require immune activation and presence of an inflammatory response to function properly and maintain a highly suppressive phenotype (Reynolds A D. et al., J Leukoc Biol 2007; 82 (5): 1083-94, Reynolds A D. et al., J Immunol 2010; 184 (5): 2261-71). Therefore, the immune activation observed here may be responsible for the induced Treg function and phenotype following sargramostim treatment. Similarly, proteomic analysis also indicated that sargramostim treatment downregulates calcineurin expression in nine pathways within 2 months of treatment. High calcineurin activity is found to drive a toxic response in the presence of high α-synuclein levels in PD (Caraveo G. et al., Proc Natl Acad Sci USA 2014; 111 (34): E3544-52).

Additionally, activation of the nuclear factor of activated T cells (NFAT) pathway which plays important roles in T cell activation and modulation of immune responses is altered (Hermann-Kleiter N. et al., Blood 2010; 115 (15): 2989-97). Most NFAT proteins are known to be regulated by calcineurin, and altered calcineurin/NFAT activation has been linked to the pathology of several neurodegenerative diseases including PD (Hogan P G. et al., Genes Dev 2003; 17 (18): 2205-32, Kipanyula M J. et al., J Aging Res 2016; 2016: 5081021). Thus, reducing calcineurin activity during sargramostim treatment suggests a mechanism by which sargramostim can provide a protective outcome in PD subjects. Secondly, the NF-kB pathway, which serves as a central mediator of inflammation, was significantly downregulated in 11 pathways. In both microglia and astroglia, activation of NF-kB, along with other proinflammatory transcription factors, leads to the transcription of several proinflammatory molecules (Liu X. et al., J Biol Chem 2002; 277 (42): 39312-9, Dasgupta S. et al., J Biol Chem 2003; 278 (25): 22424-31) that contribute or are causal to the loss of dopaminergic neurons in MPTP-intoxicated mice and PD patients (Nagatsu T. et al., J Neural Transm Suppl 2000; (60): 277-90, Mogi M. et al., Neurosci Lett 1994; 180 (2): 147-50).

Additional studies show that inhibition of NF-kB activation reduces the induction of proinflammatory molecules and significantly protects nigrostriatal neurons against MPTP-induced neurodegeneration (Ghosh A. et al., Proc Natl Acad Sci USA 2007; 104 (47): 18754-9). Therefore, reduction of NF-kB activity by sargramostim treatment may reduce the inflammation-mediated neurodegeneration and provide a consequent protective effect in PD patients. Collectively, these results indicate that treatment of PD using a therapeutic agent, such as sargramostim, can result in decreased expression of calcineurin and NF-kB in the PBLs of patients, which can be used as biomarkers for, e.g., determining the efficacy of the therapeutic treatment, or monitoring the progression of PD during treatment, or making treatment decisions.

### Limitations of the Study

As stated, this study was designed as an open-label, unblinded pilot investigation seeking to evaluate the safety and tolerability of a reduced dosing regimen for an extended time in PD. Therefore, an inherent limitation is the lack of a placebo control arm with limited subject entry. However, utilization of the subject baseline evaluations allowed for before and after treatment comparisons and timed evaluations. Secondly, the study contains a broad variability in baseline UPDRS scores, times since diagnosis, and variable immune profiles. This includes Treg and Teff numbers, lymphocyte ratios, and T cell functional assessments. To account for this variability, each subject was utilized as their own control to assess treatment-induced alterations. However, it is possible that evaluation in a more homogeneous population, such as early verse late disease, would yield variable outcomes. Lastly, the lack of female participants and start dates for anti-parkinsonian therapies during study are further limitations. All limit stratification of treatment effects, interpretation of sex differences, and evaluation of potential drug-drug effects or assessment of its therapeutic use in drug naïve subjects. Therefore, although statistically significant data is offered and these data sets are potentially meaningful, any confirmation of neurological improvements observed with treatment require careful verification in a larger, Phase II placebo-controlled study designed for drug efficacy.

### CONCLUSIONS

Taken together, and even with inherent limitations, these findings support the safety and tolerability of extended treatment with sargramostim using a low-dose and discontinuous regimen. Treatment was well-tolerated and resulted in decreased frequency and severity of adverse events compared to a higher dose and continuous regimen. The lower dose regimen also resulted in stable MDS-UPDRS Part III scores indicating no worsening of disease, and observed alterations in MDS-UPDRS Part III scores were associated with increased expression of Treg phenotypes and immunosuppressive function, thus suggesting a potential role of Treg function in diminution of disease progression. These results are intriguing and provide the basis for larger scale assessments to determine clinical efficacy of a reduced sargramostim regimen within the PD population. The study supports the notion that use of immunomodulators to induce and/or expand Tregs, shift Teff phenotype, and enhance immunosuppression in neurodegenerative disease affects neuroimmune interactions and has the potential to slow disease outcome. The study also helps to support the idea of utilizing Treg as a therapeutic target, which forms the basis for future clinical assessment. Finally, this study also supports the use of certain biomarkers, e.g., calcineurin and/or NF-kB, or one or more canonical pathways, for, e.g., determining the efficacy of the therapeutic treatment, or monitoring the progression of PD during treatment, or making treatment decisions, among other uses.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | AGTTGAATGGGGGATGTTTTTGGGATAT | Sense Primer |
| | | (5′ to 3′) |
| 2 | AATAAAATATCTACCCTCTTCTCTTCCT | Anti-sense Primer |
| | | (5′ to 3′) |
| 3 | TTGGGTTTTGTTGTTATAGTTT | Sequencing Primer |
| | | (5′ to 3′) |
| 4 | MDVFMKGLSKAKEGVVAAAEKTKQGVAEAAGKTKEGVLYV | Nitrated alpha |
| | GSKTKEGVVHGVATVAEKTKEQVTNVGGAVVTGVTAVAQK | synuclein |
| | TVEGAGSIAAATGFVKKDQLGKNEEGAPQEGILEDMPVDPDN | |
| | EAYEMPSEEGYQDYEPEA | |

What is claimed is:

1. A method of determining the efficacy of a therapeutic agent for the treatment of Parkinson's Disease (PD), the method comprising:
a) determining a baseline expression level of NF-kB and/or calcineurin in a biological sample from a subject having PD;
b) determining an expression level of NF-kB and/or calcineurin in a biological sample from the subject at one or more time points following initiation of treatment with a therapeutic agent; and
c) determining if the expression level of NF-kB and/or calcineurin at the one or more time points following initiation of treatment decreased compared to the baseline expression level.

2. A method of determining the efficacy of a therapeutic agent for the treatment of Parkinson's Disease (PD), the method comprising:
a) determining a baseline expression level of NF-kB and/or calcineurin in a biological sample from a subject having PD;
b) determining an expression level of NF-kB and/or calcineurin in a biological sample from a subject at one or more time points following initiation of treatment with a therapeutic agent; and
c) comparing the expression level of NF-kB and/or calcineurin at the one or more time points following initiation of treatment to the baseline expression level, wherein the comparison indicates a likelihood of therapeutic efficacy for the treatment of PD in the subject and/or indicates a degree of effectiveness at treating PD in the subject.

3. The method of claim 2, wherein the comparison indicates that the expression level of NF-kB and/or calcineurin at the one or more time points following initiation of treatment decreased compared to the baseline expression level.

4. The method of claim 2, wherein if the expression level of NF-kB and/or calcineurin at the one or more time points following initiation of treatment decreased compared to the baseline expression level, the comparison indicates an increased likelihood of therapeutic efficacy for the treatment of PD in the subject and/or indicates an increased degree of effectiveness at treating PD in the subject.

5. The method of claim 2, wherein if the expression level of NF-kB and/or calcineurin at the one or more time points following initiation of treatment did not decrease compared to the baseline expression level, the comparison indicates a neutral or decreased likelihood of therapeutic efficacy for the treatment of PD in the subject and/or indicates a neutral or decreased degree of effectiveness at treating PD in the subject.

6. The method of any one of claims 1-4, wherein a decreased expression level of NF-kB and/or calcineurin at the one or more time points following initiation of treatment compared to the baseline expression level indicates an increase in likelihood of therapeutic efficacy for the treatment of PD in the subject and/or indicates an increase in degree of effectiveness at treating PD in the subject.

7. The method of any one of claims 1-6, wherein the baseline expression level is determined at or at least 1 day, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment.

8. The method of any one of claims 1-7, wherein the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment.

9. The method of any one of claims 1-8, wherein the one or more time points following initiation of treatment with the therapeutic agent comprises a time point at or about 2 months following initiation of treatment.

10. The method of any one of claims 1-9, wherein the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.

11. The method of any one of claims 1-10, wherein the expression level of NF-kB and/or calcineurin is determined by an assay.

12. The method of claim 11, wherein the assay is selected from the group consisting of mass spectrometry, enzyme-linked immunosorbent assay (ELISA), western blotting, or polymerase chain reaction (PCR).

13. The method of claim 12, wherein the PCR is real-time PCR.

14. The method of any one of claims 1-13, wherein the expression level of NF-kB and/or calcineurin is decreased at the one or more time points following initiation of treatment compared to the baseline expression level.

15. A method of determining the efficacy of a therapeutic agent for the treatment of Parkinson's Disease (PD), the method comprising:
a) determining a baseline activation level of one or more canonical pathways in a biological sample from a subject having PD, wherein the one or more canonical pathways are associated with cellular immune response signaling, neuroinflammation signaling, or PD signaling pathways;
b) determining an activation level of the one or more canonical pathways in a biological sample from the subject at one or more time points following initiation of treatment with a therapeutic agent; and
c) comparing the activation level of the one or more canonical pathways at the one or more time points following initiation of treatment to the baseline expression level, wherein an increase in the activation of the one or more canonical pathways at the one or more time points following initiation of treatment to the baseline expression level indicates an increase in likelihood of therapeutic efficacy for the treatment of PD and/or indicates an increase in degree of effectiveness at treating PD in the subject.

16. The method of claim 15, wherein the comparing the activation level of the one or more canonical pathways at the one or more time points following initiation of treatment to the baseline activation level indicates a likelihood of therapeutic efficacy for the treatment of PD and/or indicates a degree of effectiveness at treating PD in the subject.

17. The method of claim 15 or claim 16, wherein if the activation level of the one or more canonical pathways at the one or more time points following initiation of treatment to the baseline activation level increased, the comparison indicates an increased likelihood of therapeutic efficacy for the treatment of PD in the subject and/or indicates an increased degree of effectiveness at treating PD in the subject.

18. The method of any one of claims 15-17, wherein an increased activation level of the one or more canonical pathways at the one or more time points following initiation of treatment as compared to the baseline activation level indicates an increase in likelihood of therapeutic efficacy for the treatment of PD in the subject and/or indicates an increase in degree of effectiveness at treating PD in the subject.

19. The method of any one of claims 15-17, wherein the activation level of the one or more canonical pathways at the one or more time points following initiation of treatment as compared to the baseline activation level increased.

20. The method of any one of claims 15-19, wherein the activation level of at least a threshold percentage of the one or more canonical pathways have increased in the biological sample at the one or more time points following initiation of the therapy compared to the baseline activation level.

21. The method of claim 20, wherein the threshold percentage is at least 50%, at least 55%, at least 60%, at least 65%, at least 70%, at least 75%, at least 80%, at least 85%, at least 90%, or at least 95%.

22. The method of any one of claims 15-21, wherein the comparison indicates an increase in likelihood of therapeutic efficacy for the treatment of PD in the subject and/or indicates an increase in degree of effectiveness at treating PD in the subject if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.

23. A method of monitoring the progression of Parkinson's Disease (PD) during treatment, the method comprising:
a) determining a baseline expression level of NF-kB and/or calcineurin in a biological sample from a subject having PD prior to initiation of a treatment comprising a therapeutic agent;
b) determining an expression level of NF-kB and/or calcineurin in a biological sample from a subject at one or more time points following initiation of the treatment; and
c) determining if the expression level of NF-kB and/or calcineurin at the one or more time points following initiation of the treatment decreased compared to the baseline expression level.

24. A method of monitoring the progression of Parkinson's Disease (PD) during treatment, the method comprising:
a) determining a baseline expression level of NF-kB and/or calcineurin in a biological sample from a subject having PD prior to initiation of a treatment comprising a therapeutic agent;
b) determining an expression level of NF-kB and/or calcineurin in a biological sample from a subject at one or more time points following initiation of the treatment; and
c) comparing the expression level of NF-kB and/or calcineurin at the one or more time points following initiation of the treatment compared to the baseline expression level, wherein the comparison indicates a likelihood of slowing, reversing, and/or halting the progression of PD in the subject.

25. The method of claim 24, wherein the comparison indicates that the expression level of NF-kB and/or calcineurin at the one or more time points following initiation of treatment decreased compared to the baseline expression level.

26. The method of claim 24, wherein if the expression level of NF-kB and/or calcineurin at the one or more time points following initiation of treatment decreased compared to the baseline expression level, the comparison indicates an increased likelihood of slowing, reversing, and/or halting the progression of PD in the subject.

27. The method of claim 24, wherein if the expression level of NF-kB and/or calcineurin at the one or more time points following initiation of treatment did not decrease compared to the baseline expression level, the comparison indicates a neutral or decreased likelihood of slowing, reversing, and/or halting the progression of PD in the subject.

28. The method of any one of claims 23-27, wherein a decreased expression level of NF-kB and/or calcineurin at the one or more time points following initiation of treatment compared to the baseline expression level indicates an increased likelihood of slowing, reversing, and/or halting the progression of PD in the subject.

29. The method of any one of claims 23-28, wherein if the expression level of NF-kB and/or calcineurin at the one or more time points following initiation of treatment decreased compared to the baseline expression level, the therapeutic agent is indicated as being effective at slowing, reversing, and/or halting the progression of PD in the subject.

30. The method of any one of claims 23-29, wherein a decreased expression level of NF-kB and/or calcineurin at the one or more time points following initiation of treatment compared to the baseline expression level indicates an increase in likelihood of slowing, reversing, and/or halting the progression of PD in the subject and/or indicates an increase in degree of effectiveness at slowing, reversing, and/or halting the progression of PD in the subject.

31. The method of any one of claims 23-30, wherein the baseline expression level is determined at or at least 1 day, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment.

32. The method of any one of claims 23-31, wherein the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment.

33. The method of any one of claims 23-32, wherein the one or more time points following initiation of treatment with the therapeutic agent comprises a time point at or about 2 months following initiation of treatment.

34. The method of any one of claims 23-33, wherein the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.

35. The method of any one of claims 23-34, wherein the expression level of NF-kB and/or calcineurin is determined by an assay.

36. The method of claim 35, wherein the assay is selected from the group consisting of mass spectrometry, enzyme-linked immunosorbent assay (ELISA), western blotting, or polymerase chain reaction (PCR).

37. The method of claim 36, wherein the PCR is real-time PCR.

38. The method of any one of claims 23-37, wherein the expression level of NF-kB and/or calcineurin is decreased at the one or more time points following initiation of treatment compared to the baseline expression level.

39. A method of monitoring the progression of Parkinson's Disease (PD) during treatment, the method comprising:
a) determining a baseline activation level of one or more canonical pathways in a biological sample from a subject having PD prior to initiation of a treatment comprising a therapeutic agent, wherein the one or more canonical pathways are associated with cellular immune response signaling, neuroinflammation signaling, or PD signaling pathways;
b) determining an activation level of the one or more canonical pathways in a biological sample from a subject at one or more time points following initiation of the treatment; and
c) comparing the activation level of the one or more canonical pathways at the one or more time points following initiation of treatment to the baseline expression level, wherein an increase in the activation of the one or more canonical pathways at the one or more time points following initiation of treatment compared to the baseline expression level indicates an increase in likelihood of slowing, reversing, and/or halting the progression of PD.

40. The method of claim 39, wherein the comparing the activation level of the one or more canonical pathways at the one or more time points following initiation of treatment to the baseline activation level indicates a likelihood of slowing, reversing, and/or halting the progression of PD in the subject.

41. The method of claim 39 or claim 40, wherein if the activation level of the one or more canonical pathways at the one or more time points following initiation of treatment to the baseline activation level increased, the comparison indicates an increased likelihood of slowing, reversing, and/or halting the progression of PD in the subject.

42. The method of any one of claims 39-41, wherein an increased activation level of the one or more canonical pathways at the one or more time points following initiation of treatment as compared to the baseline activation level indicates an increase in likelihood of slowing, reversing, and/or halting the progression of PD in the subject.

43. The method of any one of claims 39-42, wherein the activation level of the one or more canonical pathways at the one or more time points following initiation of treatment as compared to the baseline activation level increased.

44. The method of any one of claims 39-43, wherein the activation level of at least a threshold percentage of the one or more canonical pathways have increased in the biological sample at the one or more time points following initiation of the therapy compared to the baseline activation level.

45. The method of claim 44, wherein the threshold percentage is at least 50%, at least 55%, at least 60%, at least 65%, at least 70%, at least 75%, at least 80%, at least 85%, at least 90%, or at least 95%.

46. The method of any one of claims 39-45, wherein the comparison indicates an increase in likelihood of slowing, reversing, and/or halting the progression of PD in the subject if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.

47. A method for treating Parkinson's Disease (PD) in a subject, the method comprising:
a) determining a baseline expression level of NF-kB and/or calcineurin in a biological sample from a subject having PD;
b) administering a therapeutic agent for the treatment of PD to the subject;
c) determining an expression level of NF-kB and/or calcineurin in a biological sample from the subject at one or more time points following initiation of the treatment; and
d) determining if the expression level of NF-kB and/or calcineurin at the one or more time points following initiation of the treatment decreased compared to the baseline expression level, wherein a decrease in the expression level of NF-kB and/or calcineurin at the one or more time points following initiation of the treatment decreased compared to the baseline expression level indicates an increased likelihood of therapeutic efficacy in treating PD in the subject.

48. A method for treating Parkinson's Disease (PD) in a subject, the method comprising:
a) determining a baseline expression level of NF-kB and/or calcineurin in a biological sample from a subject having PD;
b) administering a therapeutic agent for the treatment of PD to the subject;
c) determining an expression level of NF-kB and/or calcineurin in a biological sample from the subject at one or more time points following initiation of the treatment; and
d) comparing the expression level of NF-kB and/or calcineurin at the one or more time points following initiation of the treatment to the baseline expression level, wherein the comparison indicates a likelihood of therapeutic efficacy in treating PD in the subject.

49. The method of claim 48, wherein the comparison indicates that the expression level of NF-kB and/or calcineurin at the one or more time points following initiation of treatment decreased compared to the baseline expression level.

50. The method of claim 48, wherein if the expression level of NF-kB and/or calcineurin at the one or more time points following initiation of treatment decreased compared to the baseline expression level, the comparison indicates an increased likelihood of therapeutic efficacy for the treatment of PD in the subject and/or indicates an increased degree of effectiveness at treating PD in the subject.

51. The method of claim 48, wherein if the expression level of NF-kB and/or calcineurin at the one or more time points following initiation of treatment did not decrease compared to the baseline expression level, the comparison indicates a neutral or decreased likelihood of therapeutic efficacy for the treatment of PD in the subject and/or indicates a neutral or decreased degree of effectiveness at treating PD in the subject.

52. The method of claim 48, wherein a decreased expression level of NF-kB and/or calcineurin at the one or more time points following initiation of the treatment compared to the baseline expression level indicates an increase in likelihood of therapeutic efficacy in treating PD in the subject.

53. The method of any one of claims 47-52, wherein the baseline expression level is determined at or at least 1 day, at or at least 1 week, at or at least 2 weeks, at or at least 3 weeks, at or at least 4 weeks, at or at least 5 weeks, at or at least 6 weeks, at or at least 7 weeks, at or at least 8 weeks, at or at least 9 weeks, at or at least 10 weeks, at or at least 11 weeks, or at or at least 12 weeks prior to initiation of the treatment.

54. The method of any one of claims 47-53, wherein the one or more time points following initiation of treatment with the therapeutic agent comprises a time point that is at or about 1 week, at or about 2 weeks, at or about 3 weeks, at or about 4 weeks, at or about 1 month, at or about 5 weeks, at or about 6 weeks, at or about 7 weeks, at or about 8 weeks, at or about 2 months, at or about 9 weeks, at or about 10 weeks, at or about 11 weeks, at or about 12 weeks, at or about 3 months, at or about 4 months, or at or about 5 months following initiation of treatment.

55. The method of any one of claims 47-54, wherein the one or more time points following initiation of treatment with the therapeutic agent comprises a time point at or about 2 months following initiation of treatment.

56. The method of any one of claims 47-55, wherein the one or more time points following initiation of treatment with the therapeutic agent comprises a time point that is between or between about 1 week and 5 months following initiation of treatment, between or between about 1 week and 4 months following initiation of treatment, between or between about 1 week and 3 months following initiation of treatment, between or between about 1 week and 2 months following initiation of treatment, between or between about 1 week and 1 month following initiation of treatment, between or between about 2 weeks and 5 months following initiation of treatment, between or between about 2 weeks and 4 months following initiation of treatment, between or between about 2 weeks and 3 months following initiation of treatment, between or between about 2 weeks and 2 months following initiation of treatment, between or between about 2 weeks and 7 weeks following initiation of treatment, or between or between about 2 weeks and 6 weeks following initiation of treatment.

57. The method of any one of claims 47-56, wherein the expression level of NF-kB and/or calcineurin is determined by an assay.

58. The method of claim 57, wherein the assay is selected from the group consisting of mass spectrometry, enzyme-linked immunosorbent assay (ELISA), western blotting, or polymerase chain reaction (PCR).

59. The method of claim 58, wherein the PCR is real-time PCR.

60. The method of any one of claims 47-59, wherein the expression level of NF-kB and/or calcineurin is decreased at the one or more time points following initiation of treatment compared to the baseline expression level.

61. The method of any one of claims 47-60, further comprising continuing treatment if the comparison indicates an increased likelihood of therapeutic efficacy.

62. The method of any one of claims 47-61, further comprising continuing treatment if the expression level of NF-kB and/or calcineurin at the one or more time points following initiation of the treatment decreased compared to the baseline expression level.

63. The method of any one of claims 47-62, further comprising discontinuing treatment if the expression level of NF-kB and/or calcineurin at the one or more time points following initiation of the treatment did not decrease compared to the baseline expression level.

64. The method of any one of claims 47-63, wherein:
(i) treatment is continued if the expression level of NF-kB and/or calcineurin have decreased in the biological sample at the one or more time points following initiation of the therapy compared to the baseline expression level; or
(ii) treatment is discontinued or altered if the expression level of NF-kB and/or calcineurin have not decreased in the biological sample at the one or more time points following initiation of the therapy compared to the baseline expression level.

65. The method of any one of claims 1-14, 23-38, and 47-64, wherein determining the baseline expression level of NF-kB and/or calcineurin occurs prior to administering the therapy.

66. A method for treating Parkinson's Disease (PD) in a subject, the method comprising:
a) determining a baseline activation level of one or more canonical pathways in a biological sample from a subject having PD, wherein the one or more canonical pathways are associated with cellular immune response signaling, neuroinflammation signaling, or PD signaling pathways;
b) administering a therapeutic agent for the treatment of PD to the subject;
c) determining an activation level of one or more canonical pathways in a biological sample from the subject at one or more time points following initiation of the treatment; and
d) comparing the activation level of the one or more canonical pathways at the one or more time points following initiation of treatment to the baseline expression level, wherein an increase in the activation level of the one or more canonical pathways indicates an increased likelihood of therapeutic efficacy.

67. The method of claim 66, further comprising continuing treatment if the activation level of the one or more canonical pathways indicates an increased likelihood of therapeutic efficacy.

68. The method of claim 66 or claim 67, wherein:
(i) treatment is continued if the activation level of the one or more canonical pathways have increased in the biological sample at the one or more time points following initiation of the therapy compared to the baseline activation level; or
(ii) treatment is discontinued or altered if the activation level of the one or more canonical pathways have not increased in the biological sample at the one or more time points following initiation of the therapy compared to the baseline activation level.

69. The method of any one of claims 66-68, wherein treatment is continued if the activation level of at least a threshold percentage of the one or more canonical pathways have increased in the biological sample at the one or more time points following initiation of the therapy compared to the baseline activation level.

70. The method of any one of claims 66-69, wherein the activation level of at least a threshold percentage of the one or more canonical pathways have increased in the biological sample at the one or more time points following initiation of the therapy compared to the baseline activation level.

71. The method of claim 69 or claim 70, wherein the threshold percentage is at least 50%, at least 55%, at least 60%, at least 65%, at least 70%, at least 75%, at least 80%, at least 85%, at least 90%, or at least 95%.

72. The method of any one of claims 66-71, wherein treatment is continued if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.

73. The method of any one of claims 66-72, wherein the activation level of the one or more canonical pathways indicates an increased likelihood of therapeutic efficacy if the activation level of at least 50%, at least 55%, at least 60%, at least 65%, at least 70%, at least 75%, at least 80%, at least 85%, at least 90%, or at least 95% of the one or more canonical pathways have increased in the biological sample at the one or more time points following initiation of the therapy compared to the baseline activation level.

74. The method of any one of claims 15-22, 39-46, and 66-73, wherein the one or more canonical pathways comprises a pathway associated with cellular immune response signaling, neuroinflammation signaling, and/or PD signaling, or any combination thereof.

75. The method of any one of claims 15-22, 39-46, and 66-74, wherein the one or more canonical pathways are selected from the group consisting of IL-8 Signaling, fMLP Signaling in Neutrophils, Role of NFAT in Regulation of the Immune Response, Fcγ Receptor-mediated Phagocytosis in Macrophages and Monocytes, CXCR4 Signaling, PKCθ Signaling in T Lymphocytes, Leukocyte Extravasation Signaling, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, CCR3 Signaling in Eosinophils, IL-3 Signaling, GM-CSF Signaling, Macropinocytosis Signaling, IL-7 Signaling Pathway, Interferon Signaling, Nur77 Signaling in T Lymphocytes, IL-9 Signaling, Antiproliferative Role of TOB in T Cell Signaling, CCR5 Signaling in Macrophages, Role of PKR in Interferon Induction and Antiviral Response, Production of Nitric Oxide and Reactive Oxygen Species in Macrophages, and iNOS Signaling.

76. The method of any one of claims 15-22, 39-46, and 66-75, wherein the one or more canonical pathways are selected from the group consisting of fMLP Signaling in Neutrophils, Role of NFAT in Regulation of the Immune Response, PKCθ Signaling in T Lymphocytes, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, IL-3 Signaling, GM-CSF Signaling, Nur77 Signaling in T Lymphocytes, IL-9 Signaling, Role of PKR in Interferon Induction and Antiviral Response, Production of Nitric Oxide and Reactive Oxygen Species in Macrophages, and iNOS Signaling.

77. The method of any one of claims 1-76, wherein the therapeutic agent is selected from the group consisting of granulocyte macrophage colony stimulating factor (GM-CSF) or an analog thereof, GM-CSF mRNA, vasoactive intestinal peptide (VIP) or an analog thereof, and VIP mRNA.

78. The method of any one of claims 1-76, wherein the therapeutic agent is an agonist of granulocyte-macrophage colony stimulating factor 2 receptor (CSF2R) or vasoactive intestinal peptide receptor 2 (VIP2R).

79. The method of claim 78, wherein the agonist is a peptide or peptide-like agonist of CSF2R or VIP2R.

80. The method of claim 78, wherein the agonist is an antibody or a fragment thereof that binds to CSF2R or VIP2R.

81. The method of any one of claims 1-76, wherein the therapeutic agent is a mimetic of GM-CSF or is a mimetic of VIP.

82. The method of any one of claims 1-76, wherein the therapeutic agent is levodopa.

83. The method of any one of claims 1-76, wherein the therapeutic agent is a dopamine receptor agonist.

84. The method of claim 77, wherein the GM-CSF or analog thereof is sargramostim.

85. The method of claim 84, wherein the sargramostim is administered subcutaneously five times per week.

86. The method of claim 77, wherein the GM-CSF or analog thereof is molgramostim.

87. The method of claim 77, wherein the VIP or analog thereof is aviptadil.

88. The method of claim 77, wherein the GM-CSF mRNA encodes sargramostim or molgramostim.

89. The method of claim 77, wherein the VIP mRNA encodes aviptadil.

90. The method of any one of claims 1-76, wherein the therapeutic agent is an agent that reacts with and/or induces molecules that react with a GM-CSF receptor or a VIP receptor.

91. The method of claim 90, wherein the GM-CSF receptor is CSF2R and/or the VIP receptor is VIP2R.

92. The method of any one of claims 1-76, wherein the therapeutic agent is an immunogen that induces a humoral immune response against at least one abnormal protein of PD.

93. The method of claim 92, wherein the immunogen is nitrated alpha synuclein or a fragment thereof.

94. The method of claim 93, wherein the immunogen is a nitrated alpha synuclein fragment, and wherein the nitrated alpha synuclein fragment is a carboxy terminal fragment consisting of the carboxy terminal 20 amino acids of alpha synuclein up to the carboxy terminal 70 amino acids of alpha synuclein.

95. The method of any one of claims 92-94, wherein the immunogen is human nitrated alpha synuclein or a fragment thereof.

96. The method of any one of claims 92-95, wherein the immunogen is comprised in a composition that further comprises at least one adjuvant that stimulates regulatory T cells.

97. The method of claim 96, wherein the adjuvant is selected from the group consisting of VIP, vitamin D, GM-CSF, and transforming growth factor beta (TGFβ).

98. The method of any one of claims 93-97, wherein the nitrated alpha synuclein comprises the amino acid sequence of SEQ ID NO: 4, or comprises an amino acid sequence having at least 90%, 91%, 92%, 93%, 94%, 95%, 96%, 97%, 98%, or 99% sequence identity to the amino acid sequence of SEQ ID NO: 4.

99. The method of any one of claims 93-97, wherein the nitrated alpha synuclein fragment comprises amino acid residues 101-140 of SEQ ID NO: 4, or comprises an amino acid sequence having at least 90%, 91%, 92%, 93%, 94%, 95%, 96%, 97%, 98%, or 99% sequence identity to amino acid residues 101-140 of the amino acid sequence of SEQ ID NO: 4.

100. The method of any one of claims 96-99, wherein the composition further comprises a pharmaceutically acceptable carrier.

101. The method of any one of claims 1-100, wherein the biological sample comprises peripheral blood lymphocytes.

102. The method of any one of claims 1-101, wherein the biological sample comprises T cells.

103. The method of any one of claims 1-102, wherein the biological sample is obtained from the subject by leukapheresis.

104. A composition for detecting NF-kB and/or calcineurin expression in a biological sample, wherein the composition comprises a kit for a mass spectrometry analysis, an ELISA assay, a western blotting assay, or a PCR reaction.

105. The composition of claim 104, wherein the kit comprises a reagent for an ELISA assay.

106. The composition of claim 105, wherein the kit for the ELISA assay comprises a multi-well sample plate that is coated with immobilized capture antibodies that bind to NF-kB and/or calcineurin; detection antibodies covalently linked to an enzyme wherein the detection antibodies also bind to NF-kB and/or calcineurin; a colored or fluorescent product that will be catalyzed by the enzyme attached to the detection antibody; and appropriate buffers.

107. Biomarkers for monitoring the progression of Parkinson's Disease (PD) and/or the effectiveness of therapeutics for the treatment of PD, wherein the biomarkers are NF-kB and calcineurin; or are one or more canonical pathways associated with cellular immune response signaling, neuroinflammation signaling, or PD signaling pathways.

108. The method of claim 107, wherein the one or more canonical pathways are selected from the group consisting of IL-8 Signaling, fMLP Signaling in Neutrophils, Role of NFAT in Regulation of the Immune Response, Fcγ Receptor-mediated Phagocytosis in Macrophages and Monocytes, CXCR4 Signaling, PKCθ Signaling in T Lymphocytes, Leukocyte Extravasation Signaling, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, CCR3 Signaling in Eosinophils, IL-3 Signaling, GM-CSF Signaling, Macropinocytosis Signaling, IL-7 Signaling Pathway, Interferon Signaling, Nur77 Signaling in T Lymphocytes, IL-9 Signaling, Antiproliferative Role of TOB in T Cell Signaling, CCR5 Signaling in Macrophages, Role of PKR in Interferon Induction and Antiviral Response, Production of Nitric Oxide and Reactive Oxygen Species in Macrophages, and iNOS Signaling.

109. The method of claim 107 or claim 108, wherein the one or more canonical pathways are selected from the group consisting of fMLP Signaling in Neutrophils, Role of NFAT in Regulation of the Immune Response, PKCθ Signaling in T Lymphocytes, CD28 Signaling in T Helper Cells, iCOS-iCOSL Signaling in T Helper Cells, Calcium-induced T Lymphocyte Apoptosis, Natural Killer Cell Signaling, PI3K Signaling in B Lymphocytes, IL-3 Signaling, GM-CSF Signaling, Nur77 Signaling in T Lymphocytes, IL-9 Signaling, Role of PKR in Interferon Induction and Antiviral Response, Production of Nitric Oxide and Reactive Oxygen Species in Macrophages, and iNOS Signaling.

110. A therapeutic agent for use in the treatment of Parkinson's Disease (PD) in a subject, wherein the therapeutic agent is for use in a method comprising:
a) determining a baseline expression level of NF-kB and/or calcineurin in a biological sample from a subject having PD;
b) determining an expression level of NF-kB and/or calcineurin in a biological sample from the subject at one or more time points following administration of the treatment; and
c) comparing the expression level of NF-kB and/or calcineurin at the one or more time points following initiation of the treatment to the baseline expression level, wherein the comparison indicates a likelihood of therapeutic efficacy in treating PD in the subject.

111. Use of a therapeutic agent in the treatment of Parkinson's Disease (PD) in a subject, wherein the use comprises:
a) determining a baseline expression level of NF-kB and/or calcineurin in a biological sample from a subject having PD;
b) determining an expression level of NF-kB and/or calcineurin in a biological sample from the subject at one or more time points following administration of the therapeutic agent; and
c) comparing the expression level of NF-kB and/or calcineurin at the one or more time points following initiation of the treatment to the baseline expression level, wherein the comparison indicates a likelihood of therapeutic efficacy in treating PD in the subject.

112. A therapeutic agent for use in the treatment of Parkinson's Disease (PD) in a subject, wherein the therapeutic agent is for use in a method comprising:
a) determining a baseline activation level of one or more canonical pathways in a biological sample from a subject having PD, wherein the one or more canonical pathways are associated with cellular immune response signaling, neuroinflammation signaling, or PD signaling pathways;
b) determining an activation level of one or more canonical pathways in a biological sample from the subject at one or more time points following administration of the treatment; and
c) comparing the activation level of one or more canonical pathways in a biological sample from the subject at the one or more time points following initiation of the treatment to the baseline expression level, wherein the comparison indicates a likelihood of therapeutic efficacy in treating PD in the subject.

113. Use of a therapeutic agent in the treatment of Parkinson's Disease (PD) in a subject, wherein the use comprises:
a) determining a baseline activation level of one or more canonical pathways in a biological sample from a subject having PD, wherein the one or more canonical pathways are associated with cellular immune response signaling, neuroinflammation signaling, or PD signaling pathways;
b) determining an activation level of one or more canonical pathways in a biological sample from the subject at one or more time points following administration of the treatment; and
c) comparing the activation level of one or more canonical pathways in the biological sample from the subject at the one or more time points following initiation of the treatment to the baseline expression level, wherein the comparison indicates a likelihood of therapeutic efficacy in treating PD in the subject.


SEQUENCE LISTING (4 sequences)

SEQ ID NO: 1 - DNA, 28 bp, Artificial Sequence
  Sense Primer
agttgaatgggggatgtttttgggatat

SEQ ID NO: 2 - DNA, 28 bp, Artificial Sequence
  Anti-sense Primer
aataaaatatctaccctcttctcttcct

SEQ ID NO: 3 - DNA, 22 bp, Artificial Sequence
  Sequencing Primer
ttgggttttgttgttatagttt

SEQ ID NO: 4 - protein, 140 aa, Artificial Sequence
  Nitrated alpha synuclein
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA